(19) United States
(12) Patent Application Publication
(10) Pub. No.: US 2024/0216427 A1
(43) Pub. Date: Jul. 4, 2024

(54) CHIMERIC ANTIGEN RECEPTORS TARGETING SPLICE VARIANTS OF THE EXTRACELLULAR MATRIX PROTEINS TENASCIN C (TNC) AND PROCOLLAGEN 11A1 (COL11A1)

(71) Applicant: St. Jude Children's Research Hospital, Inc., Memphis, TN (US)

(72) Inventors: Stephen GOTTSCHALK, Germantown, TN (US); Jessica WAGNER, Memphis, TN (US); Elizabeth WICKMAN, Memphis, TN (US); Timothy Isham SHAW, Memphis, TN (US); Jinghui ZHANG, Germantown, TN (US)

(73) Assignee: St. Jude Children's Research Hospital, Inc., Memphis, TN (US)

(21) Appl. No.: 18/270,675

(22) PCT Filed: Dec. 29, 2021
(86) PCT No.: PCT/US2021/065445

§ 371 Date: Jun. 30, 2023

Related U.S. Application Data
(60) Provisional application No. 63/132,121, filed on Dec. 30, 2020.

(51) Int. Cl. A61K 35/17; A61K 39/00; A61P 35/02; C07K 14/705; C07K 14/725; C07K 16/18

(52) U.S. Cl. CPC A61K 35/17; A61K 39/4611; A61K 39/4631; A61P 35/02; C07K 14/7051; C07K 14/70521; C07K 16/18; A61K 2239/13; A61K 2239/21; C07K 2317/565; C07K 2317/622; C07K 2319/02; C07K 2319/03

(57) ABSTRACT

The application provides chimeric antigen receptors (CARs) that target splice variants of the extracellular matrix proteins tenascin C (TNC) and procollagen 11A1 (Col11A1), and their uses in tumor immunotherapy. The application also provides polynucleotides and vectors that encode the chimeric antigen receptors, as well as host cells comprising the chimeric antigen receptors. The application also provides methods for preparing host cells comprising the chimeric antigen receptors and methods for treating patients using the modified host cells.

### CROSS-REFERENCE TO RELATED APPLICATIONS

This application claims priority to U.S. Provisional Application No. 63/132,121, filed, Dec. 30, 2020, the disclosure of which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Dec. 15, 2021, is named 243734_000164_SL.txt and is 205,807 bytes in size.

### FIELD OF THE INVENTION

The application relates to chimeric antigen receptors (CARs), particularly CARs targeting splice variants of the extracellular matrix proteins tenascin C (TNC) and procollagen 11A1 (Col11A1), and their uses in tumor immunotherapy (e.g., adoptive cell therapy). The application further relates to therapeutic cells that express such CARs and methods for treating patients using the CAR-expressing therapeutic cells.

### BACKGROUND

Cancer cells often express splice variants since their spliceosome is altered. One type of splice variants that are overexpressed in various cancers are the splice variants of procollagen 11A1 (Col11A1). Procollagen alpha 1(XI) chain, encoded by the COL11A1 gene, forms a procollagen molecule with two other collagen chains (alpha 2(XI) and alpha 1(II)). The procollagen molecule is then enzymatically processed in cells to form collagen XI fibers. Col11A1 is thought to play a role in cell invasiveness. It is also indicated to play a role in breast cancer. Splice variants of human procollagen 11A1 have been identified in various cancer types such as rhabdomyosarcoma and osteosarcoma. The VAR sub-domain in the N-terminal propeptide of Col11A1 has different sequences and characteristics according to alternative splicing, combining additional exons (e.g., exons 6, 7, 8, and/or 9) of the gene (Barneo L et al., 41st Congress of the European Society for Surgical Research-ESSR. Bologna; Italy, Vollmar Brigitte (ed) Medimond, International Proceedings; 27-35; Garcia-Ocana M et al., Int J Oncol. 2012 May; 40(5):1447-54).

Another type of splice variant commonly expressed in cancer cells is known as oncofetal tenascin C. Tenascin C (TNC) is a large hexameric glycoprotein of the extracellular matrix which modulates cellular adhesion. It is secreted into tumor stroma and binds to the cell surface through integrins. It is involved in processes such as cell proliferation and cell migration and is associated with changes in tissue architecture as occurring during morphogenesis and embryogenesis as well as under tumorigenesis or angiogenesis. Several isoforms of tenascin C can be generated as a result of alternative splicing which may lead to the inclusion of (multiple) domains in the central part of this protein. In the oncofetal tenascin C isoform, additional exons are present including an extra domain C of tenascin C (Giblin S P and Midwood K S. Cell Adh Migr. 2015; 9(1-2):48-82). The C domain of tenascin C is undetectable in most normal adult tissues, but is overexpressed in high-grade astrocytomas (Carnemolla B et al., Am J Pathol 1999; 154:1345-1352) and other tumor types.

Many solid tumors and brain tumors depend on stromal extracellular matrix and neovasculature for survival, and, therefore, splice variants of extracellular matrix proteins, such as Col11A1 and TNC, can be generalizable targets that are not limited to a specific tumor type. However, chimeric antigen receptor (CAR)-based therapies targeting such splice variants are currently underdeveloped. Accordingly, there exists a need for CAR-based therapies targeting the splice variants of the extracellular matrix proteins for the treatment of cancer.

### SUMMARY OF THE INVENTION

As specified in the Background section above, there is a great need in the art for CARs that target splice variants of the extracellular matrix proteins in solid tumors and brain tumors. Splice variants of procollagen 11A1 (Col11A1) and tenascin C (TNC) are ideal targets, as they are overexpressed in various cancer types. The present application addresses these and other needs.

In one aspect, provided herein is a polynucleotide encoding a chimeric antigen receptor (CAR) comprising:
(a) an extracellular target-binding domain comprising a binding moiety which binds to a procollagen 11A1 (Col11A1) splice variant,
(b) a transmembrane domain, and
(c) a cytoplasmic domain comprising a signaling domain.

In some embodiments, the binding moiety binds to exon 6 within the VAR sub-domain of a propeptide of Col11A1.

In some embodiments, the binding moiety is an antibody, or a fragment thereof, or a peptide that binds to the Col11A1 splice variant. In some embodiments, the anti-Col11A1 antibody fragment is a single chain variable fragment (scFv), Fab, Fab′, F(ab′)2, Fv fragment, dsFv diabody, VHH, VNAR, single-domain antibody (sdAb) or nanobody, dAb fragment, Fd′ fragment, or Fd fragment.

In some embodiments, the anti-Col11A1 antibody fragment is an anti-Col11A1 scFv. In some embodiments, the anti-Col11A1 scFv is derived from antibody 1E8.33. In some embodiments, the anti-Col11A1 scFv comprises a heavy chain complementarity determining region 1 (HCDR1), a HCDR2, and a HCDR3 as defined in the heavy chain variable domain (VH) comprising the amino acid sequence SEQ ID NO: 64, or an amino acid sequence having at least 80% identity thereof, and/or a light chain complementarity determining region 1 (LCDR1), a LCDR2, and a LCDR3 as defined in the light chain variable domain (VL) comprising the amino acid sequence SEQ ID NO: 68, or an amino acid sequence having at least 80% identity thereof. In some embodiments, the anti-Col11A1 scFv comprises a HCDR1 comprising the amino acid sequence of SEQ ID NO: 114, a HCDR2 comprising the amino acid sequence of SEQ ID NO: 115, and a HCDR3 comprising the amino acid sequence of SEQ ID NO: 116; and/or a LCDR1 comprising the amino acid sequence of SEQ ID NO: 117, a LCDR2 comprising the amino acid sequence of YTS, and a LCDR3 comprising the amino acid sequence SEQ ID NO: 118. In some embodiments, the anti-Col11A1 scFv comprises a heavy chain variable domain (VH) comprising the amino acid sequence of SEQ ID NO: 64, or an amino acid sequence having at least 80% identity thereof. In some embodiments, the nucleotide sequence encoding the anti-Col11A1 scFv VH comprises the sequence of SEQ ID NO: 65, or a nucleotide sequence having at least 80% identity thereof. In some embodiments, the anti-Col11A1 scFv comprises a light chain variable domain (VL) comprising the amino acid sequence of SEQ ID NO: 68, or an amino acid sequence having at least 80% identity thereof. In some embodiments, the nucleotide sequence encoding the anti-Col11A1 scFv VL comprises the sequence of SEQ ID NO: 69, or a nucleotide sequence having at least 80% identity thereof.

In some embodiments, the anti-Col11A1 scFv further comprises a linker between the VH and VL. In some embodiments, the linker sequence comprises the amino acid sequence GGGGSGGGGSGGGGS ((G₄S)₃; SEQ ID NO: 10), GGGGS (SEQ ID NO: 13), (G₄S)₂ (SEQ ID NO: 72), (G₄S)₄ (SEQ ID NO: 73), KESGSVSSEQLAQFRSLD (SEQ ID NO: 74), EGKSSGSGSESKST (SEQ ID NO: 75), EGKSSGSGSESKSTQ (SEQ ID NO: 76), GSTSGSGKSSEGKG (SEQ ID NO: 77), SSADDAKKDDAKKDDAKKDDAKKDG (SEQ ID NO: 78), EGKSSGSGSESKVD (SEQ ID NO: 79), ESGSVSSEELAFRSLD (SEQ ID NO: 80), EGKSSGSGSESKST (SEQ IDNO: 81), or EGKSSGSGSESKSTQ (SEQ IDNO: 82), or an amino acid sequence having at least 80% identity thereof. In some embodiments, the linker sequence comprises the amino acid sequence GGGGSGGGGSGGGGS (SEQ ID NO: 10) or GGGGS (SEQ ID NO: 13), or an amino acid sequence having at least 80% identity thereof. In some embodiments, the nucleotide encoding the linker sequence comprises SEQ ID NO: 11, SEQ ID NO: 12, SEQ ID NO: 14, or a nucleotide sequence having at least 80% identity thereof.

In some embodiments, the anti-Col11A1 scFv comprises the amino acid sequence of SEQ ID NO: 4, or an amino acid sequence having at least 80% identity thereof. In some embodiments, the nucleotide sequence encoding the anti-Col11A1 scFv comprises the sequence of SEQ ID NO: 5, or a nucleotide sequence having at least 80% identity thereof.

In some embodiments, the extracellular target-binding domain further comprises a leader sequence. In some embodiments, the leader sequence comprises the amino acid sequence of SEQ ID NO: 1, or an amino acid sequence having at least 80% identity thereof. In some embodiments, the nucleotide sequence encoding the leader sequence comprises the sequence of SEQ ID NO: 2 or SEQ ID NO: 3, or a nucleotide sequence having at least 80% identity thereof.

In some embodiments, the extracellular target-binding domain further comprises a hinge domain. In some embodiments, the hinge domain is derived from IgG1, IgG2, IgG3, IgG4, CD28, or CD8a. In some embodiments, the hinge domain is derived from IgG1, optionally comprising the amino acid sequence of SEQ ID NO: 15, or an amino acid sequence having at least 80% identity thereof. In some embodiments, the nucleotide sequence encoding the hinge domain comprises the sequence of SEQ ID NO: 16, or a nucleotide sequence having at least 80% identity thereof.

In some embodiments, the extracellular binding domain comprises the amino acid sequence of SEQ ID NO: 36, or an amino acid sequence having at least 80% identity thereof. In some embodiments, the nucleotide sequence encoding the extracellular binding domain comprises the sequence of SEQ ID NO: 37, or a nucleotide sequence having at least 80% identity thereof.

In some embodiments, the transmembrane domain is derived from CD28, CD8α, CD4, or CD3ζ. In some embodiments, the transmembrane domain is derived from CD28, optionally comprising the amino acid sequence of SEQ ID NO: 21, or an amino acid sequence having at least 80% identity thereof. In some embodiments, the nucleotide sequence encoding the transmembrane domain comprises the sequence of SEQ ID NO: 22, or a nucleotide sequence having at least 80% identity thereof.

In some embodiments, the signaling domain is derived from CD3ζ, DAP10, DAP12, Fc epsilon receptor I γ chain (FCER1G), CD3δ, CD3γ, CD3γ, CD226, or CD79A. In some embodiments, the signaling domain is derived from CD3ζ, optionally comprising the amino acid sequence of SEQ ID NO: 29, or an amino acid sequence having at least 80% identity thereof. In some embodiments, the nucleotide sequence encoding the signaling domain comprises the sequence of SEQ ID NO: 30, or a nucleotide sequence having at least 80% identity thereof.

In some embodiments, the cytoplasmic domain further comprises one or more costimulatory domain. In some embodiments, the costimulatory domain is derived from CD28, CD27, CD40, CD134, CD137, CD226, CD79A, ICOS, MyD88, IL-2Rβ, or the STAT3-binding YXXQ. In some embodiments, the costimulatory domain is derived from CD28, optionally comprising the amino acid sequence of SEQ ID NO: 27, or an amino acid sequence having at least 80% identity thereof. In some embodiments, the nucleotide sequence encoding the costimulatory domain comprises the sequence of SEQ ID NO: 28, or a nucleotide sequence having at least 80% identity thereof.

In some embodiments, the cytoplasmic domain comprises the amino acid sequence of SEQ ID NO: 48, or an amino acid sequence having at least 80% identity thereof. In some embodiments, the nucleotide sequence encoding the cytoplasmic domain comprises the sequence of SEQ ID NO: 49, or a nucleotide sequence having at least 80% identity thereof.

In some embodiments, the polynucleotide further encodes at least one additional polypeptide. In some embodiments, the sequence encoding the CAR is operably linked to the sequence encoding the at least one additional polypeptide via a sequence encoding a self-cleaving peptide and/or an internal ribosomal entry site (IRES). In some embodiments, the self-cleaving peptide is a 2A peptide. In some embodiments, the 2A peptide is T2A, P2A, E2A, or F2A peptide. In some embodiments, the 2A peptide is a T2A peptide. In some embodiments, the T2A peptide comprises the amino acid sequence of SEQ ID NO: 31, or an amino acid sequence having at least 80% sequence identity thereof. In some embodiments, the sequence encoding the T2A peptide comprises the nucleotide sequence of SEQ ID NO: 32, or a nucleotide sequence having at least 80% sequence identity thereof. In some embodiments, the at least one polypeptide is a transduced host cell selection marker, an in vivo tracking marker, a cytokine, or a safety switch gene. In some embodiments, the transduced host cell selection marker is a truncated CD19 (tCD19) polypeptide. In some embodiments, the tCD19 comprises the amino acid sequence of SEQ ID NO: 33, or an amino acid sequence having at least 80% sequence identity thereof. In some embodiments, the nucleotide sequence encoding the tCD19 comprises the nucleotide sequence of SEQ ID NO: 34 or SEQ ID NO: 35, or a nucleotide sequence having at least 80% sequence identity thereof.

In some embodiments, the CAR comprises the amino acid sequence SEQ ID NO: 52, or an amino acid sequence having at least 80% identity thereof. In some embodiments, the polynucleotide comprises the nucleotide sequence SEQ ID NO: 53, or a nucleotide sequence having at least 80% identity thereof.

In various embodiments, the polynucleotide of any one of those described above is a DNA molecule. In various embodiments, the polynucleotide of any one of those described above is an RNA molecule.

In another aspect, provided herein is a recombinant vector comprising the polynucleotide of any one of those described above. In some embodiments, the vector is a viral vector. In some embodiments, the viral vector is a retroviral vector, a lentiviral vector, an adenoviral vector, an adeno-associated virus vector, an alphaviral vector, a herpes virus vector, or a vaccinia virus vector.

In some embodiments, the viral vector is a retroviral vector. In some embodiments, the vector is a non-viral vector.

In another aspect, provided herein is a chimeric antigen receptor (CAR) encoded by the polynucleotide of any one of those described above.

In another aspect, provided herein is an isolated host cell comprising the polynucleotide of any one of those described above or the recombinant vector of any one of those described above.

In another aspect, provided herein is an isolated host cell comprising the CAR described above. In some embodiments, the host cell is an immune cell. In some embodiments, the immune cell is a T-cell, a NK cell, or a macrophage. In some embodiments, the T-cell is selected from a CD8+ T-cell, a CD4+ T-cell, a cytotoxic T-cell, an ap T-cell receptor (TCR) T-cell, an invariant natural killer T (iNKT) cell, a γδ T-cell, a memory T-cell including memory stem T-cell (TSCM), a naïve T-cell, an effector T-cell, a T-helper cell, and a regulatory T-cell (Treg). In some embodiments, the host cell has been activated and/or expanded ex vivo. In some embodiments, the host cell is an allogeneic cell. In some embodiments, the host cell is an autologous cell. In some embodiments, the host cell is isolated from a subject having a tumor, wherein one or more cells of the tumor express a Col11A1 splice variant. In some embodiments, the tumor is a solid tumor. In some embodiments, the tumor is selected from acute lymphoblastic leukemia, acute myeloid leukemia, adult solid tumors and brain tumors, adrenal gland tumors, anal cancer, bile duct cancer, bladder cancer, blood cancers, bone cancer, bowel cancer, brain tumors, breast cancer, cancer of unknown primary, cancer spread to bone, cancer spread to brain, cancer spread to liver, cancer spread to lung, carcinoid, cervical cancer, children's cancers, colorectal cancer, chronic lymphocytic leukemia, chronic myeloid leukemia, colorectal cancer, ear cancer, endometrial cancer, eye cancer, follicular dendritic cell sarcoma, gallbladder cancer, gastric cancer, gastro esophageal junction cancers, germ cell tumors, gestational trophoblastic disease, glioma, glioblastoma, gynecological cancer, hairy cell leukemia, head and neck squamous cell carcinoma, high grade gliomas, Hodgkin lymphoma, Kaposi's sarcoma, kidney cancer, large bowel and rectal neuroendocrine tumors, laryngeal cancer, leukemia, Linitis plastica of the stomach, liver cancer, low grade gliomas, lung cancer, lung neuroendocrine tumors (NETs), lymphoma, malignant schwannoma, mediastinal germ cell tumors, melanoma, men's cancer, merkel cell skin cancer, mesothelioma, molar pregnancy, mouth and oropharyngeal cancer, myeloma, nasal and paranasal sinus cancer, nasopharyngeal cancer, neuroblastoma, neuroendocrine tumors, neuroendocrine tumors of the pancreas, non-Hodgkin lymphoma, non-Hodgkin lymphoma in children, esophageal cancer, oral squamous cell carcinoma, ovarian cancer, pancreatic cancer, pediatric solid tumors and brain tumors, penile cancer, persistent trophoblastic disease and choriocarcinoma, pheochromocytoma, prostate cancer, pseudomyxoma peritonei, rare cancers, rectal cancer, renal cancer, retinoblastoma, salivary gland cancer, secondary cancer, signet cell cancer, skin cancer, small bowel cancer, small bowel neuroendocrine tumors, soft tissue sarcoma, stomach cancer, stomach neuroendocrine tumors, testis cancer, thymus gland tumors, thyroid cancer, tongue cancer, tonsil cancer, tumors of the adrenal gland, unknown primary cancer, urothelial, uterine cancer, vaginal cancer, vulval cancer, Wilms' tumor, and womb cancer. In some embodiments, the host cell is derived from a blood, marrow, tissue, or a tumor sample.

In another aspect, provided herein is a pharmaceutical composition comprising the host cell described above and a pharmaceutically acceptable carrier and/or excipient.

In another aspect, provided herein is a method of generating the isolated host cell described above, said method comprising genetically modifying the host cell with the polynucleotide described above or the recombinant vector described above. In some embodiments, the vector is a viral vector and the genetic modification is conducted by a transduction using said vector. In some embodiments, the genetic modification is conducted ex vivo. In some embodiments, the method further comprises activation and/or expansion of the host cell ex vivo before, after and/or during said genetic modification.

In another aspect, provided herein is a method for killing a tumor cell expressing a Col11A1 splice variant, said method comprising contacting said cell with the host cell(s) described above or the pharmaceutical composition described above.

In another aspect, provided herein is a method for treating a tumor in a subject in need thereof, wherein one or more cells of the tumor express a Col11A1 splice variant, said method comprising administering to the subject a therapeutically effective amount of the host cells described above or the pharmaceutical composition described above. In some embodiments, the tumor is a solid tumor. In some embodiments, the tumor is selected from acute lymphoblastic leukemia, acute myeloid leukemia, adult solid tumors and brain tumors, adrenal gland tumors, anal cancer, bile duct cancer, bladder cancer, blood cancers, bone cancer, bowel cancer, brain tumors, breast cancer, cancer of unknown primary, cancer spread to bone, cancer spread to brain, cancer spread to liver, cancer spread to lung, carcinoid, cervical cancer, children's cancers, colorectal cancer, chronic lymphocytic leukemia, chronic myeloid leukemia, colorectal cancer, ear cancer, endometrial cancer, eye cancer, follicular dendritic cell sarcoma, gallbladder cancer, gastric cancer, gastro esophageal junction cancers, germ cell tumors, gestational trophoblastic disease, glioma, glioblastoma, gynecological cancer, hairy cell leukemia, head and neck squamous cell carcinoma, high grade gliomas, Hodgkin lymphoma, Kaposi's sarcoma, kidney cancer, large bowel and rectal neuroendocrine tumors, laryngeal cancer, leukemia, Linitis plastica of the stomach, liver cancer, low grade gliomas, lung cancer, lung neuroendocrine tumors (NETs), lymphoma, malignant schwannoma, mediastinal germ cell tumors, melanoma, men's cancer, merkel cell skin cancer, mesothelioma, molar pregnancy, mouth and oropharyngeal cancer, myeloma, nasal and paranasal sinus cancer, nasopharyngeal cancer, neuroblastoma, neuroendocrine tumors, neuroendocrine tumors of the pancreas, non-Hodgkin lymphoma, non-Hodgkin lymphoma in children, esophageal cancer, oral squamous cell carcinoma, ovarian cancer, pancreatic cancer, pediatric solid tumors and brain tumors, penile cancer, persistent trophoblastic disease and choriocarcinoma, pheochromocytoma, prostate cancer, pseudomyxoma peritonei, rare cancers, rectal cancer, renal cancer, retinoblastoma, salivary gland cancer, secondary cancer, signet cell cancer, skin cancer, small bowel cancer, small bowel neuroendocrine tumors, soft tissue sarcoma, stomach cancer, stomach neuroendocrine tumors, testis cancer, thymus gland tumors, thyroid cancer, tongue cancer, tonsil cancer, tumors of the adrenal gland, unknown primary cancer, urothelial, uterine cancer, vaginal cancer, vulval cancer, Wilms' tumor, and womb cancer.

In some embodiments of the treatment method described above, the method further comprises:
a) isolating T-cells, NK cells, iNKT cells or macrophages from the subject or generating T-cells, NK cells, iNKT cells or macrophages from stem cells including induced pluripotent stem cells (iPS cells);
b) genetically modifying said T-cells, NK cells, iNKT cells, macrophages or stem cells including iPS cells ex vivo with the polynucleotide of any one of those described above or the vector of any one of those described above;
c) optionally, expanding and/or activating said T-cells, NK cells, iNKT cells or macrophages before, after or during step b); and
d) introducing the genetically modified T-cells, NK cells, iNKT cells or macrophages into the subject.

In various embodiments of the methods described above, the subject is human. In some embodiments, the subject is an adult. In some embodiments, the subject is a child.

In various embodiments described above, the Col11A1 splice variant contains at least exon 6 within the VAR sub-domain of a propeptide of Col11A1.

In another aspect, provided herein is a polynucleotide encoding a chimeric antigen receptor (CAR) comprising:
(a) an extracellular target-binding domain comprising a binding moiety which binds to a C domain of tenascin C (C.TNC) splice variant,
(b) a transmembrane domain, and
(c) a cytoplasmic domain comprising a signaling domain.

In some embodiments, the binding moiety is an anti-C.TNC antibody, or fragment thereof, or a peptide. In some embodiments, the anti-C.TNC antibody fragment is a single chain variable fragment (scFv), Fab, Fab′, F(ab′)2, Fv fragment, dsFv diabody, VHH, VNAR, single-domain antibody (sdAb) or nanobody, dAb fragment, Fd′ fragment, or Fd fragment.

In some embodiments, the anti-C.TNC antibody fragment is an anti-C.TNC scFv. In some embodiments, the anti-C.TNC scFv is derived from antibody G11. In some embodiments, the anti-C.TNC scFv comprises a heavy chain complementarity determining region 1 (HCDR1), a HCDR2, and a HCDR3 as defined in the heavy chain variable domain (VH) comprising the amino acid sequence SEQ ID NO: 66, or an amino acid sequence having at least 80% identity thereof; and/or a light chain complementarity determining region 1 (LCDR1), a LCDR2, and a LCDR3 as defined in the light chain variable domain (VL) comprising the amino acid sequence SEQ ID NO: 70, or an amino acid sequence having at least 80% identity thereof. In some embodiments, the anti-C.TNC scFv comprises a HCDR1 comprising the amino acid sequence of SEQ ID NO: 119, a HCDR2 comprising the amino acid sequence of SEQ ID NO: 120, and a HCDR3 comprising the amino acid sequence of SEQ ID NO: 121; and/or a LCDR1 comprising the amino acid sequence of SEQ ID NO: 122, a LCDR2 comprising the amino acid sequence of SEQ ID NO: 123, and a LCDR3 comprising the amino acid sequence SEQ ID NO: 124. In some embodiments, the anti-C.TNC scFv comprises a heavy chain variable domain (VH) comprising the amino acid sequence of SEQ ID NO: 66, or an amino acid sequence having at least 80% identity thereof. In some embodiments, the nucleotide sequence encoding the anti-anti-C.TNC scFv VH comprises the sequence of SEQ ID NO: 67, or a nucleotide sequence having at least 80% identity thereof. In some embodiments, the anti-C.TNC scFv comprises a light chain variable domain comprising the amino acid sequence of SEQ ID NO: 70, or an amino acid sequence having at least 80% identity thereof. In some embodiments, the nucleotide sequence encoding the anti-C.TNC scFv VL comprises the sequence of SEQ ID NO: 71, or a nucleotide sequence having at least 80% identity thereof.

In some embodiments, the anti-C.TNC scFv further comprises a linker between the VH and VL. In some embodiments, the linker sequence comprises the amino acid sequence GGGGSGGGGSGGGGS ((G₄S)₃; SEQ ID NO: 10), GGGGS (SEQ ID NO: 13), (G₄S)₂ (SEQ ID NO: 72), (G₄S)₄ (SEQ ID NO: 73), KESGSVSSEQLAQFRSLD (SEQ ID NO: 74), EGKSSGSGSESKST (SEQ ID NO: 75), EGKSSGSGSESKSTQ (SEQ ID NO: 76), GSTSGSGKSSEGKG (SEQ ID NO: 77), SSADDAKKDDAKKDDAKKDDAKKDG (SEQ ID NO: 78), EGKSSGSGSESKVD (SEQ ID NO: 79), ESGSVSSEELAFRSLD (SEQ ID NO: 80), EGKSSGSGSESKST (SEQ IDNO: 81), or EGKSSGSGSESKSTQ (SEQ IDNO: 82), or an amino acid sequence having at least 80% identity thereof. In some embodiments, the linker sequence comprises the amino acid sequence GGGGSGGGGSGGGGS (SEQ ID NO: 10) or GGGGS (SEQ ID NO: 13), or an amino acid sequence having at least 80% identity thereof. In some embodiments, the nucleotide encoding the linker sequence comprises SEQ ID NO: 11, SEQ ID NO: 12, or SEQ ID NO: 14, or a nucleotide sequence having at least 80% identity thereof.

In some embodiments, the anti-C.TNC scFv comprises the amino acid sequence of SEQ ID NO: 6, or an amino acid sequence having at least 80% identity thereof. In some embodiments, the nucleotide sequence encoding the anti-C.TNC scFv comprises the sequence of SEQ ID NO: 7, or a nucleotide sequence having at least 80% identity thereof. In some embodiments, the anti-C.TNC scFv comprises the amino acid sequence of SEQ ID NO: 8, or an amino acid sequence having at least 80% identity thereof. In some embodiments, the nucleotide sequence encoding the anti-C.TNC scFv comprises the sequence of SEQ ID NO: 9, or a nucleotide sequence having at least 80% identity thereof.

In some embodiments, the extracellular target-binding domain further comprises a leader sequence. In some embodiments, the leader sequence comprises the amino acid sequence of SEQ ID NO: 1, or an amino acid sequence having at least 80% identity thereof. In some embodiments, the nucleotide sequence encoding the leader sequence comprises the sequence of SEQ ID NO: 2 or SEQ ID NO: 3, or a nucleotide sequence having at least 80% identity thereof.

In some embodiments, the extracellular target-binding domain further comprises a hinge domain. In some embodiments, the hinge domain is derived from IgG1, IgG2, IgG3, IgG4, CD28, or CD8α. In some embodiments, the hinge domain is derived from IgG1, optionally comprising the amino acid sequence of SEQ ID NO: 15, or an amino acid sequence having at least 80% identity thereof. In some embodiments, the nucleotide sequence encoding the hinge domain comprises the sequence of SEQ ID NO: 16, or a nucleotide sequence having at least 80% identity thereof. In some embodiments, the hinge domain is derived from IgG4, optionally comprising the amino acid sequence of SEQ ID NO: 17, or an amino acid sequence having at least 80% identity thereof. In some embodiments, the nucleotide sequence encoding the hinge domain comprises the sequence of SEQ ID NO: 18, or a nucleotide sequence having at least 80% identity thereof. In some embodiments, the hinge domain is derived from CD8α, optionally comprising the amino acid sequence of SEQ ID NO: 19, or an amino acid sequence having at least 80% identity thereof. In some embodiments, the nucleotide sequence encoding the hinge domain comprises the sequence of SEQ ID NO: 20, or a nucleotide sequence having at least 80% identity thereof.

In some embodiments, the extracellular binding domain comprises the amino acid sequence SEQ ID NO: 38, 40, 42, 44, or 46, or an amino acid sequence having at least 80% identity thereof. In some embodiments, the nucleotide sequence encoding the extracellular binding domain comprises the sequence SEQ ID NO: 39, 41, 43, 45, or 47, or a nucleotide sequence having at least 80% identity thereof.

In some embodiments, the transmembrane domain is derived from CD28, CD8α, CD4, or CD3ζ. In some embodiments, the transmembrane domain is derived from CD28, optionally comprising the amino acid sequence of SEQ ID NO: 21, or an amino acid sequence having at least 80% identity thereof. In some embodiments, the nucleotide sequence encoding the transmembrane domain comprises the sequence of SEQ ID NO: 22, or a nucleotide sequence having at least 80% identity thereof. In some embodiments, the transmembrane domain is derived from CD8α, optionally comprising the amino acid sequence of SEQ ID NO: 23, or an amino acid sequence having at least 80% identity thereof. In some embodiments, the nucleotide sequence encoding the transmembrane domain comprises the sequence of SEQ ID NO: 24, or a nucleotide sequence having at least 80% identity thereof. In some embodiments, the transmembrane domain is derived from CD3ζ, optionally comprising the amino acid sequence of SEQ ID NO: 25, or an amino acid sequence having at least 80% identity thereof. In some embodiments, the nucleotide sequence encoding the transmembrane domain comprises the sequence of SEQ ID NO: 26, or a nucleotide sequence having at least 80% identity thereof.

In some embodiments, the signaling domain is derived from CD3ζ, DAP10, DAP12, Fc epsilon receptor I γ chain (FCER1G), CD3δ, CD3ε, CD3γ, CD226, or CD79A. In some embodiments, the signaling domain is derived from CD3ζ, optionally comprising the amino acid sequence of SEQ ID NO: 29, or an amino acid sequence having at least 80% identity thereof. In some embodiments, the nucleotide sequence encoding the signaling domain comprises the sequence of SEQ ID NO: 30, or a nucleotide sequence having at least 80% identity thereof.

In some embodiments, the cytoplasmic domain further comprises one or more costimulatory domain. In some embodiments, the costimulatory domain is derived from CD28, CD27, CD40, CD134, CD137, CD226, CD79A, ICOS, MyD88, IL-2Rβ, or the STAT3-binding YXXQ. In some embodiments, the costimulatory domain is derived from CD28, optionally comprising the amino acid sequence of SEQ ID NO: 27, or an amino acid sequence having at least 80% identity thereof. In some embodiments, the nucleotide sequence encoding the costimulatory domain comprises the sequence of SEQ ID NO: 28, or a nucleotide sequence having at least 80% identity thereof.

In some embodiments, the cytoplasmic domain comprises the amino acid sequence of SEQ ID NO: 48, or an amino acid sequence having at least 80% identity thereof. In some embodiments, the nucleotide sequence encoding the cytoplasmic domain comprises the sequence of SEQ ID NO: 49, or a nucleotide sequence having at least 80% identity thereof. In some embodiments, the cytoplasmic domain comprises the amino acid sequence of SEQ ID NO: 50, or an amino acid sequence having at least 80% identity thereof. In some embodiments, the nucleotide sequence encoding the cytoplasmic domain comprises the sequence of SEQ ID NO: 51, or a nucleotide sequence having at least 80% identity thereof.

In some embodiments, the polynucleotide further encodes at least one additional polypeptide. In some embodiments, the sequence encoding the CAR is operably linked to the sequence encoding the at least one additional polypeptide via a sequence encoding a self-cleaving peptide and/or an internal ribosomal entry site (IRES). In some embodiments, the self-cleaving peptide is a 2A peptide. In some embodiments, the 2A peptide is T2A, P2A, E2A, or F2A peptide. In some embodiments, the 2A peptide is a T2A peptide. In some embodiments, the T2A peptide comprises the amino acid sequence of SEQ ID NO: 31, or an amino acid sequence having at least 80% sequence identity thereof. In some embodiments, the sequence encoding the T2A peptide comprises the nucleotide sequence of SEQ ID NO: 32, or a nucleotide sequence having at least 80% sequence identity thereof. In some embodiments, the at least one polypeptide is a transduced host cell selection marker, an in vivo tracking marker, a cytokine, or a safety switch gene. In some embodiments, the transduced host cell selection marker is a truncated CD19 (tCD19) polypeptide. In some embodiments, the tCD19 comprises the amino acid sequence of SEQ ID NO: 33, or an amino acid sequence having at least 80% sequence identity thereof. In some embodiments, the nucleotide sequence encoding the tCD19 comprises the nucleotide sequence of SEQ ID NO: 34 or SEQ ID NO: 35, or a nucleotide sequence having at least 80% sequence identity thereof.

In some embodiments, the anti-C.TNC CAR comprises the amino acid sequence SEQ ID NO: 54, 56, 58, 60, 62, or 125, or an amino acid sequence having at least 80% identity thereof. In some embodiments, the polynucleotide comprises the nucleotide sequence SEQ ID NO: 55, 57, 59, 61, 63, or 126, or a nucleotide sequence having at least 80% identity thereof.

In various embodiments, the polynucleotide of any one of those described above is a DNA molecule. In various embodiments, the polynucleotide of any one of those described above is an RNA molecule.

In another aspect, provided herein is a recombinant vector comprising the polynucleotide of any one of those described above. In some embodiments, the vector is a viral vector. In some embodiments, the viral vector is a retroviral vector, a lentiviral vector, an adenoviral vector, an adeno-associated virus vector, an alphaviral vector, a herpes virus vector, or a vaccinia virus vector. In some embodiments, the viral vector is a retroviral vector. In some embodiments, the vector is a non-viral vector.

In another aspect, provided herein is a chimeric antigen receptor (CAR) encoded by the polynucleotide of any one of those described above.

In another aspect, provided herein is an isolated host cell comprising the polynucleotide of any one of those described above or the recombinant vector of any one of those described above.

In another aspect, provided herein is an isolated host cell comprising the CAR described above. In some embodiments, the host cell is an immune cell. In some embodiments, the immune cell is a T-cell, a NK cell, or a macrophage. In some embodiments, the T-cell is selected from a CD8+ T-cell, a CD4+ T-cell, a cytotoxic T-cell, an ap T-cell receptor (TCR) T-cell, an invariant natural killer T (iNKT) cell, a γδ T-cell, a memory T-cell including memory stem T-cell (TSCM), a naïve T-cell, an effector T-cell, a T-helper cell, and a regulatory T-cell (Treg). In some embodiments, the host cell has been activated and/or expanded ex vivo. In some embodiments, the host cell is an allogeneic cell. In some embodiments, the host cell is an autologous cell. In some embodiments, the host cell is isolated from a subject having a tumor, wherein one or more cells of the tumor express C.TNC. In some embodiments, the tumor is a solid tumor. In some embodiments, the tumor is selected from breast cancer, brain tumors such as, but not limited to, glioblastoma, high grade gliomas, low grade gliomas, head and neck cancers, liver cancers, lung cancers, melanoma, ovarian cancer, pancreatic cancer, prostate cancer, renal cancer, urothelial cancer, carcinoid, cervical cancers, colorectal cancer, endometrial cancer, lymphoma, skin cancer, stomach cancer, testis cancer, thyroid cancer and urothelial cancer. In some embodiments, the host cell is derived from a blood, marrow, tissue, or a tumor sample.

In another aspect, provided herein is a pharmaceutical composition comprising the host cell of any one of those described above and a pharmaceutically acceptable carrier and/or excipient.

In another aspect, provided herein is a method of generating the isolated host cell of any one of those described above, said method comprising genetically modifying the host cell with the polynucleotide of any one of those described above or the recombinant vector of any one of those described above. In some embodiments, the vector is a viral vector and the genetic modification is conducted by a transduction using said vector. In some embodiments, the genetic modification is conducted ex vivo. In some embodiments, the method further comprises activation and/or expansion of the host cell ex vivo before, after and/or during said genetic modification.

In another aspect, provided herein is a method for killing a tumor cell expressing C.TNC, said method comprising contacting said cell with the host cell(s) of any one of those described above or the pharmaceutical composition described above.

In another aspect, provided herein is a method for treating a tumor in a subject in need thereof, wherein one or more cells of the tumor express C.TNC, said method comprising administering to the subject a therapeutically effective amount of the host cells of any one of those described above or the pharmaceutical composition described above. In some embodiments, the tumor is a solid tumor. In some embodiments, the tumor is selected from brain tumors such as, but not limited to, glioblastoma, high grade gliomas, low grade gliomas, head and neck cancers, liver cancers, lung cancers, melanoma, ovarian cancer, pancreatic cancer, prostate cancer, renal cancer, urothelial cancer, carcinoid, cervical cancers, colorectal cancer, endometrial cancer, lymphoma, skin cancer, stomach cancer, testis cancer, thyroid cancer and urothelial cancer.

In some embodiments of the treatment method described above, the method comprising:
a) isolating T-cells, NK cells, iNKT cells or macrophages from the subject or generating T-cells, NK cells, iNKT cells or macrophages from stem cells including induced pluripotent stem cells (iPS cells);
b) genetically modifying said T-cells, NK cells, iNKT cells, macrophages or stem cells including iPS cells ex vivo with the polynucleotide of any one of those described above or the vector of any one of those described above;
c) optionally, expanding and/or activating said T-cells, NK cells, iNKT cells or macrophages before, after or during step b); and
d) introducing the genetically modified T-cells, NK cells, iNKT cells or macrophages into the subject.

In various embodiments of the methods described above, the subject is human. In some embodiments, the subject is an adult. In some embodiments, the subject is a child.

In another aspect, provided herein is an isolated host cell comprising the polynucleotide or the recombinant vector encoding an anti-Col11A1 CAR described above; and the polynucleotide or the recombinant vector encoding an anti-C.TNC CAR described above.

In another aspect, provided herein is an isolated host cell comprising an anti-Col11A1 CAR described above and an anti-C.TNC CAR described above. In some embodiments, the host cell is an immune cell. In some embodiments, the immune cell is a T-cell, a NK cell, or a macrophage. In some embodiments, the T-cell is selected from a CD8+ T-cell, a CD4+ T-cell, a cytotoxic T-cell, an ap T-cell receptor (TCR) T-cell, an invariant natural killer T (iNKT) cell, a γδ T-cell, a memory T-cell including memory stem T-cell (TSCM), a naïve T-cell, an effector T-cell, a T-helper cell, and a regulatory T-cell (Treg). In some embodiments, the host cell has been activated and/or expanded ex vivo. In some embodiments, the host cell is an allogeneic cell. In some embodiments, the host cell is an autologous cell.

In another aspect, provided herein is a pharmaceutical composition comprising the host cell comprising an anti-Col11A1 CAR and the host cell comprising an anti-Col11A1 CAR, or the host cell comprising an anti-Col11A1 CAR and an anti-Col11A1 CAR; and a pharmaceutically acceptable carrier and/or excipient.

In another aspect, provided herein is a method of generating the isolated host cell comprising an anti-Col11A1 CAR and an anti-Col11A1 CAR, said method comprising genetically modifying the host cell with a polynucleotide or recombinant vector encoding an anti-Col11A1 CAR described above, and a polynucleotide or recombinant vector encoding an anti-C.TNC CAR described above. In some embodiments, the vector is a viral vector and the genetic modification is conducted by a transduction using said vectors. In some embodiments, the genetic modification is conducted ex vivo. In some embodiments, the method further comprises activation and/or expansion of the host cell ex vivo before, after and/or during said genetic modification.

In another aspect, provided herein is a method for killing a tumor cell expressing a Col11A1 splice variant and/or C.TNC, said method comprising contacting said cell with the host cell(s) of any one of those described above or the pharmaceutical composition described above.

In another aspect, provided herein is a method for treating a tumor in a subject in need thereof, wherein one or more cells of the tumor express a Col11A1 splice variant and/or C.TNC, said method comprising administering to the subject a therapeutically effective amount of the host cells of any one of those described above or the pharmaceutical composition described above.

### BRIEF DESCRIPTION OF THE DRAWINGS

FIG. 1A-1C show Col11A1 splice variant expression in pediatric cancer. Schematic representation of Col11A1 exons is shown in FIG. 1A. For heatmap visualization of Collagen Type XI Alpha 1 Chain (Col11A1) exon expression (FIG. 1B), RNA sequencing (RNAseq) reads were processed by two-pass STAR mapping followed by high-throughput sequencing (HTseq) exon quantification. Gene abundance was measured as the number of fragments per kilobase of transcripts per million mapped reads (FPKM), and rank normalized on a heatmap. Each cell of the heatmap shows the sample median for each pediatric tumor and normal (non-cancerous) tissue. RNAseq from pediatric solid and brain tumors were used to quantify tumor exon expression. Genotype-tissue expression (GTEx) RNAseq samples were used to quantify exon expression in normal (non-cancerous) tissue. FIG. 1C shows a schematic representation of Col11A1 exons and the exon that is recognized by an exemplary Col11A1-CAR described herein is indicated with an arrow.

FIG. 2 shows Col11A1 expression by quartiles from the Pediatric Cancer Genome Project. Pediatric tumor samples were characterized based RNA expression of the C domain of TNC as either high expression (Q4: greater than 75%), medium-high expression (Q3: 50-70%), medium-low expression (Q2: 25-50%), or low expression (Q1: less than 25%). HGG: high grade glioma, EPD: ependymoma, LGG: low grade glioma, MB: medulloblastoma, RHB: rhabdomyosarcoma, OS: osteosarcoma, ACT: adrenocortical carcinoma, MEL: melanoma, RB: retinoblastoma, INF: infant all, ERG: B-ALL with ERG alterations, PHALL: Philadelphia like acute lymphoblastic leukemia, MLL: mixed lineage leukemia.

FIGS. 3A-3C show the generation of Col11A1-CAR T cells. A retroviral vector was designed encoding an COL11A1-specific CAR (Col11A1-CAR) using a COL11A1-specific scFv (1E8.33) that has shown tumor specificity in human imaging studies, a CD28 hinge/transmembrane domain (CD28H/TM), and a CD28.ζ signaling domain (FIG. 3A). COL11A1-CAR T-cells were generated by retroviral transduction of CD3/CD28-activated T-cells in the presence of IL-7 (10 ng/ml) and IL-15 (10 ng/ml). CAR expression was detected on transduced T-cells by fluorescence-activated cell sorting (FACS) analysis for truncated CD19 (tCD19; FIG. 3B) and anti-F(ab)′ (FIG. 3C; n=4 donors, ***p<0.001, ****p<0.0001, 2-way ANOVA).

FIGS. 4A-4B show Col11A1 CAR recognition and killing of Col11A1+ tumor cells in vitro. To evaluate COL11A1-CAR T-cells recognition and killing of Col11A1+ tumor cells in vitro, multiple cell lines were tested (U87: high grade glioma, A549: lung cancer, MDA-MB-468 and MCF7: breast cancer, A673: Ewing's sarcoma). Cytolytic activity of COL11A1-CAR and non-transduced (NT) T-cells was determined by standard MTS assay at 4:1 E:T ratio for 3 days. COL11A1 induced cell death in breast cancer and Ewing's sarcoma cell lines (FIG. 4A). To measure interferon-gamma (IFNγ) secretion, 5×10⁵ tumor cells and 1×10⁶ T-cells were co-cultured in wells of a 24-well tissue culture plate. After 24 hours, the cell culture media was harvested and IFNγ production (pg/mL) was determined by enzyme-linked immunosorbent assay (ELISA), as displayed in FIG. 4B (n=3 donors, ***p<0.001, ****p<0.0001, 2-way ANOVA).

FIGS. 5A-5B show Col11A1 recognition and killing of Col11A1+ tumor cells in vivo. A673 Ewing's sarcoma cells (2×10⁶ cells) were injected subcutaneously (s.c.) into immunodeficient NOD scid gamma (NSG) mice, and on day 10, mice received a single intravenous injection of 1×10⁶ CoL11A1-CAR T cells or NT T-cells. Tumor growth was measured (mm³) by serial caliper (FIG. 5A). Kaplan Meier survival analysis shows statistically significant advantage (FIG. 5B; n=5 *p<0.05 Log-rank [Mantel-Cox test]).

FIG. 6A-6C show TNC C domain (C.TNC) expression in pediatric cancer. Schematic representation of tenascin C (TNC) exons is shown in FIG. 6A. For heatmap visualization of TNC exon expression (FIG. 6B), RNAseq reads were processed by two-pass STAR mapping followed by HTseq exon quantification. Gene abundance was measured as the number of fragments per kilobase of transcripts per million mapped reads (FPKM), and rank normalized on a heatmap. Each cell of the heatmap shows the sample median for each pediatric tumor and normal (non-cancerous) tissue. RNAseq from pediatric solid and brain tumors were used to quantify tumor exon expression. GTEx RNAseq samples were used to quantify exon expression in normal (non-cancerous) tissue. FIG. 6C shows a schematic representation of C.TNC exons and the exon that is recognized by the exemplary C.TNC-CARs described herein is indicated with an arrow.

FIG. 7 shows C.TNC expression by quartiles from the Pediatric Cancer Genome Project. Pediatric tumor samples were characterized based RNA expression of the C domain of TNC as either high expression (Q4: greater than 75%), medium-high expression (Q3: 50-70%), medium-low expression (Q2: 25-50%), or low expression (Q1: less than 25%). HGG: high grade glioma, EPD: ependymoma, LGG: low grade glioma, MB: medulloblastoma, RHB: rhabdomyosarcoma, OS: osteosarcoma, MEL: melanoma, CMF: chondromyxofibroma, RB: retinoblastoma, INF: infant all, ERG: B-ALL with ERG alterations, PHALL: Philadelphia like acute lymphoblastic leukemia, MLL: mixed lineage leukemia.

FIGS. 8A-8B show C.TNC as a target for CART cells. Schematic of CART cells specific for the C domain of TNC targeting variant-expressing tumor cells is shown in FIG. 8A. A retroviral vector was designed encoding a C domain-specific CAR (C.TNC-CAR), utilizing the scFv G11, a CD28hinge/transmembrane domain (CD28H/TM), and a CD28.ζ signaling domain (FIG. 8B).

FIGS. 9A-9D show C.TNC-CAR T cell recognition and killing of C.TNC+ tumor cells in vitro. To evaluate C.TNC-CAR T cells recognition and killing of C.TNC+ tumor cells, multiple cell lines were tested in vitro. To measure IFNγ secretion, 5×10⁵ tumor cells were co-cultured with 1×10⁶ T cells. After 48 hours, the cell culture media was harvested, and cytokine production was determined by ELISA (n=3 donors, **<0.05, ****<0.0001, 2-way ANOVA). NT: Non-transduced T cells, A673: Ewing's sarcoma, LM7: osteosarcoma (FIG. 9A). To measure GM-CSF secretion, 5×10⁵ tumor cells were co-cultured with 1×10⁶ T cells. After 72 hours, the cell culture media was harvested, and cytokine production was determined by ELISA (FIG. 9B; n=2 donors, **<0.05, ****<0.0001, 2-way ANOVA). Cytolytic activity of C.TNC-CAR T cells was determined by evaluating luminescence produced by A673.ffluc tumor cells 72 hours post co-culturing T cells and tumor cells (FIG. 9C; n=3 donors, ****<0.0001, 2-way ANOVA). Cytolytic activity of C.TNC-CAR T cells was determined by evaluating luminescence produced by LM7.ffluc tumor cells expressing firefly luciferase 72 hours post co-culturing T cells and tumor cells (FIG. 9D; n=3 donors, ****<0.0001, 2-way ANOVA).

FIGS. 10A-10B show C.TNC-CAR T cells killing of C.TNC+A673 cells in vivo. A673 Ewing's sarcoma cells (2×10⁶ cells) were injected subcutaneously (s.c.) into immunodeficient NOD scid gamma (NSG) mice, and on day 9, mice received a single intravenous injection of 1×10⁶ sorted T cells expressing firefly luciferase (fflu). Mice received C.TNC-CAR T cells or NT T-cells. Schematic of experimental setup is shown in FIG. 10A. Tumor growth was measured (mm³) by serial caliper (FIG. 10B; n=5, *<0.05, **<0.01, 2-way ANOVA).

FIG. 11 shows additional C.TNC-CAR designs. Additional retroviral constructs were generated by cloning the G11 scFv into different CAR expression cassettes.

FIGS. 12A-12N show the amino acid sequences and nucleotide sequences for the exemplary CARs of the present disclosure.

### DETAILED DESCRIPTION

The present disclosure provides chimeric antigen receptors (CARs) and T-cells or other lymphocytes expressing said CARs that target antigens located on the target tumor cell and/or the extracellular matrix (ECM) within the tumor micro-environment (TME) with special focus on procollagen 11A1 (Col11A1) and tenascin C (TNC).

CAR-expressing cells targeting the splice variants of Col11A1 or TNC could potentially target a broad range of solid and brain tumors. Col11A1 or TNC splice variants are expressed in pediatric and adult tumors. In addition, one concern of targeting solid tumors with CAR-based cell therapy is “on target/off cancer” toxicity; CAR-expressing cells targeting the splice variants of Col11A1 or TNC have the potential to reduce the risk of “on target/off cancer” toxicity.

CARs are primarily comprised of 1) an antigen-binding moiety, such as but not limited to a single-chain variable fragment (scFv) derived from an antigen-specific monoclonal antibody, and 2) a lymphocyte activation domain, such as but not limited to the ζ-chain from the T-cell receptor CD3. These two regions are fused together via a transmembrane domain. A hinge domain is usually required to provide more flexibility and accessibility between the antigen-binding moiety and the transmembrane domain. Upon transduction, the lymphocyte expresses the CAR on its surface, and upon contact and ligation with the target antigen, it signals through the lymphocyte activation domain (e.g., CD3ζ chain) inducing cytotoxicity and cellular activation.

CAR constructs may also include co-stimulatory polypeptides to boost the CAR-induced immune response. The most commonly used co-stimulating molecules include CD28 and 4-1BB, which promotes both T-cell proliferation and cell survival. Another example of co-stimulatory domains is a MyD88/CD40 molecule that can be used with or without the use of a separate dimerization agent. Additional CAR constructs may also include three signaling domains (e.g., CD3ζ, CD28, and 4-1BB), which further improves lymphocyte cell survival and efficacy.

In certain embodiments, the polynucleotide encoding the CAR is further operably linked to a second gene. In certain embodiments, the second gene encodes a truncated CD19 (tCD19) polypeptide.

### Definitions

The term “chimeric antigen receptor” or “CAR” as used herein is defined as a cell-surface receptor comprising an extracellular target-binding domain, a transmembrane domain, and a cytoplasmic domain comprising a lymphocyte activation domain and optionally at least one co-stimulatory signaling domain, all in a combination that is not naturally found together on a single protein. This particularly includes receptors wherein the extracellular domain and the cytoplasmic domain are not naturally found together on a single receptor protein. The chimeric antigen receptors of the present disclosure can be used with lymphocyte such as T-cells and natural killer (NK) cells.

The terms “T cell” and “T lymphocyte” are interchangeable and used synonymously herein. As used herein, T-cell includes thymocytes, naïve T lymphocytes, immature T lymphocytes, mature T lymphocytes, resting T lymphocytes, or activated T lymphocytes. A T-cell can be a T helper (Th) cell, for example a T helper 1 (Th1) or a T helper 2 (Th2) cell. The T-cell can be a helper T-cell (HTL; CD4+ T-cell) CD4+ T-cell, a cytotoxic T-cell (CTL; CD8+ T-cell), a tumor infiltrating cytotoxic T-cell (TIL; CD8+ T-cell), CD4+CD8+ T-cell, or any other subset of T-cells. Other illustrative populations of T-cells suitable for use in particular embodiments include naïve T-cells and memory T-cells. Also included are “NKT cells”, which refer to a specialized population of T-cells that express a semi-invariant ap T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T-cells (γδ T-cells),” which refer to a specialized population that to a small subset of T-cells possessing a distinct TCR on their surface, and unlike the majority of T-cells in which the TCR is composed of two glycoprotein chains designated α- and β-TCR chains, the TCR in γδ T-cells is made up of a γ-chain and a δ-chain. γδ T-cells can play a role in immunosurveillance and immunoregulation, and were found to be an important source of IL-17 and to induce robust CD8+ cytotoxic T-cell response. Also included are “regulatory T-cells” or “Tregs” refers to T-cells that suppress an abnormal or excessive immune response and play a role in immune tolerance. Tregs cells are typically transcription factor Foxp3-positive CD4+ T cells and can also include transcription factor Foxp3-negative regulatory T-cells that are IL-10-producing CD4+ T cells.

The terms “natural killer cell” and “NK cell” are used interchangeable and used synonymously herein. As used herein, NK cell refers to a differentiated lymphocyte with a CD 16+CD56+ and/or CD57+ TCR-phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “antigen-binding moiety” refers to a target-specific binding element that may be any ligand that binds to the antigen of interest or a polypeptide or fragment thereof, wherein the ligand is either naturally derived or synthetic. Examples of antigen-binding moieties include, but are not limited to, antibodies; polypeptides derived from antibodies, such as, for example, single chain variable fragments (scFv), Fab, Fab′, F(ab′)2, and Fv fragments; polypeptides derived from T-cell receptors, such as, for example, TCR variable domains; secreted factors (e.g., cytokines, growth factors) that can be artificially fused to signaling domains (e.g., “zytokines”); and any ligand or receptor fragment (e.g., CD27, NKG2D) that binds to the antigen of interest. Combinatorial libraries could also be used to identify peptides binding with high affinity to the therapeutic target.

Terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5a, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12. In certain embodiments, the host cell is autologous. In certain embodiments, the host cell is allogenic.

Host cells of the present disclosure include T-cells and natural killer cells that contain the DNA or RNA sequences encoding the CAR and express the CAR on the cell surface. Such host cells may be used for enhancing T-cell activity, natural killer cell activity, treatment of tumors, and treatment of autoimmune disease.

The terms “activation” or “stimulation” means to induce a change in their biologic state by which the cells (e.g., T-cells and NK cells) express activation markers, produce cytokines, proliferate and/or become cytotoxic to target cells. All these changes can be produced by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T-cell and/or NK cell proliferation and/or upregulation or downregulation of key molecules.

The term “proliferation” refers to an increase in cell division, either symmetric or asymmetric division of cells. The term “expansion” refers to the outcome of cell division and cell death.

The term “differentiation” refers to a method of decreasing the potency or proliferation of a cell or moving the cell to a more developmentally restricted state.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular or transmembrane.

The term “transfection” means the introduction of a “foreign” (i.e., extrinsic or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

The term “transduction” means the introduction of a foreign nucleic acid into a cell using a viral vector.

The terms “genetically modified” or “genetically engineered” refers to the addition of extra genetic material in the form of DNA or RNA into a cell.

As used herein, the term “derivative” or “variant” in the context of proteins or polypeptides (e.g., CAR constructs or domains thereof) refer to: (a) a polypeptide that has at least 40%, 45%, 50%, 55%, 60%, 65%, 70%, 75%, 80%, 85%, 90%, 95%, 98% or 99% sequence identity to the polypeptide it is a derivative or variant of, (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a derivative or variant of, (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a derivative or variant of, (d) a polypeptide encoded by nucleic acids can hybridize under high, moderate or typical stringency hybridization conditions to nucleic acids encoding the polypeptide it is a derivative or variant of; (e) a polypeptide encoded by a nucleotide sequence that can hybridize under high, moderate or typical stringency hybridization conditions to a nucleotide sequence encoding a fragment of the polypeptide, it is a derivative or variant of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a derivative or variant of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

The terms “vector”, “cloning vector” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In certain embodiments, the vector is a viral vector such as, but not limited to, viral vector is an adenoviral, adeno-associated, alphaviral, herpes, lentiviral, retroviral, or vaccinia vector.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the term “operatively linked” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

By “enhance” or “promote” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T-cell expansion, activation, effector function, persistence, and/or an increase in tumor cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower” or “lessen” or “reduce” or “abate” refers generally to the ability of composition contemplated herein to produce, elicit, or cause a lesser physiological response (i.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

If aspects of the disclosure are described as “comprising” a feature, or versions there of (e.g., comprise), embodiments also are contemplated “consisting of” or “consisting essentially of” the feature.

The practice of the present disclosure employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

### Chimeric Antigen Receptors

In certain aspects, the disclosure provides CARs that target splice variants of extracellular matrix proteins, such as procollagen 11A1 (Col11A1) and tenascin C (TNC), located on the target tumor cell and/or the extracellular matrix (ECM) within the tumor microenvironment to allow for targeting of the tumor cells and/or ECM (e.g., neovasculature, stromal cells such as cancer associated fibroblasts, etc.).

In certain aspects, the present disclosure provides a polynucleotide encoding a CAR comprising: (a) an extracellular target-binding domain comprising a binding moiety which binds to a procollagen 11A1 (Col11A1) splice variant, (b) a transmembrane domain, and (c) a cytoplasmic domain comprising a signaling domain. In some embodiments, the Col11A1 splice variant contains at least exon 6 within the VAR sub-domain of the propeptide of Col11A1. In some embodiments, the Col11A1 splice variant contains exons 6 and 7 within the VAR sub-domain of the propeptide of Col11A1. In some embodiments, the Col11A1 splice variant contains exons 6, 7, 8 and 9 within the VAR sub-domain of the propeptide of Col11A1. In some embodiments, the binding moiety binds to exon 6 within the VAR sub-domain of the propeptide of Col11A1.

In certain aspects, the present disclosure provides a polynucleotide encoding a CAR comprising: (a) an extracellular target-binding domain comprising a binding moiety which binds to a tenascin C (TNC) splice variant, (b) a transmembrane domain, and (c) a cytoplasmic domain comprising a signaling domain. In some embodiments, the TNC splice variant contains at least the C domain of TNC (C.TNC). In some embodiments, the TNC splice variant contains exons A1, A2, A3, A4, B, AD2, AD1, C, and D of TNC. In some embodiments, the binding moiety binds to the C domain of TNC (C.TNC).

In certain aspects, the present disclosure provides a CAR comprising: (a) an extracellular target-binding domain comprising a binding moiety which binds to a procollagen 11A1 (Col11A1) splice variant, (b) a transmembrane domain, and (c) a cytoplasmic domain comprising a signaling domain. In some embodiments, the Col11A1 splice variant contains at least exon 6 within the VAR sub-domain of the propeptide of Col11A1. In some embodiments, the Col11A1 splice variant contains exons 6 and 7 within the VAR sub-domain of the propeptide of Col11A1. In some embodiments, the Col11A1 splice variant contains exons 6, 7, 8 and 9 within the VAR sub-domain of the propeptide of Col11A1. In some embodiments, the binding moiety binds to exon 6 within the VAR sub-domain of the propeptide of Col11A1.

In certain aspects, the present disclosure provides a CAR comprising: (a) an extracellular target-binding domain comprising a binding moiety which binds to a tenascin C (C.TNC) splice variant, (b) a transmembrane domain, and (c) a cytoplasmic domain comprising a signaling domain. In some embodiments, the TNC splice variant contains at least the C domain of TNC (C.TNC). In some embodiments, the TNC splice variant contains exons A1, A2, A3, A4, B, AD2, AD1, C, and D of TNC. In some embodiments, the binding moiety binds to the C domain of TNC (C.TNC).

### Extracellular Target-Binding Domain

In certain aspects, CARs of the present disclosure comprise an extracellular target-binding domain, wherein the extracellular target-binding domain comprises an antigen-binding moiety.

The choice of antigen-binding moiety depends upon the type and number of antigens that define the surface of a target cell. For example, the antigen-binding moiety may be chosen to recognize an antigen that acts as a cell surface marker on target cells associated with a particular disease state. In certain embodiments, the CARs of the present disclosure can be genetically modified to target a tumor antigen of interest by way of engineering a desired antigen-binding moiety that specifically binds to an antigen (e.g., on a tumor cell). Non-limiting examples of cell surface markers that may act as targets for the antigen-binding moiety in the CAR of the disclosure include those associated with tumor cells.

Examples of antigens that may be targeted by the extracellular target-binding domains include, but are not limited to, splice variants of extracellular matrix proteins, such as tenascin C and procollagen 11A1 (Col11A1).

In certain embodiments, the antigen that is targeted by the extracellular target-binding domain is a procollagen 11A1 (Col11A1) splice variant. In some embodiments, the Col11A1 splice variant contains at least exon 6 within the VAR sub-domain of the propeptide of Col11A1. In some embodiments, the Col11A1 splice variant contains exons 6 and 7 within the VAR sub-domain of the propeptide of Col11A1. In some embodiments, the Col11A1 splice variant contains exons 6, 7, 8, and 9 within the VAR sub-domain of the propeptide of Col11A1. In some embodiments, the binding moiety binds to exon 6 within the VAR sub-domain of the propeptide of Col11A1.

In certain embodiments, the antigen that is targeted by the extracellular target-binding domain is a tenascin C (TNC) splice variant. In some embodiments, the TNC splice variant contains at least the C domain of TNC (C.TNC). In some embodiments, the TNC splice variant contains exons A1, A2, A3, A4, B, AD2, AD1, C, and D of TNC. In some embodiments, the binding moiety binds to the C domain of TNC (C.TNC).

In certain embodiments, the antigen-binding moiety can be monomeric or multimeric (e.g., homodimeric or heterodimeric), or associated with multiple proteins in a non-covalent complex. In some embodiments, the antigen-binding moiety comprises an antigen-binding peptide, polypeptide or functional variant thereof that binds to an antigen. In some embodiments, the antigen-binding polypeptide is an antibody or an antibody fragment that binds to an antigen. Antigen-binding moieties may comprise antibodies and/or antibody fragments such as monoclonal antibodies, multispecific antibodies, chimeric antibodies, single-chain Fvs (scFv), single chain antibodies, Fab fragments, F(ab′) fragments, disulfide-linked Fvs (sdFv), intrabodies, minibodies, single domain antibody variable domains, nanobodies (VHHs), diabodies and anti-idiotypic (anti-Id) antibodies (including, e.g., anti-Id antibodies to antigen-specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the antigen-binding moiety is a single-chain Fv (scFv). In some embodiments, the scFv comprises a linker between the VH and VL. Non-limiting examples of the linker sequence that may be used in the scFvs described herein include, GGGGSGGGGSGGGGS ((G₄S)₃; SEQ ID NO: 10), GGGGS (SEQ ID NO: 13), (G₄S)₂ (SEQ ID NO: 72), (G₄S)₄ (SEQ ID NO: 73), KESGSVSSEQLAQFRSLD (SEQ ID NO: 74), EGKSSGSGSESKST (SEQ ID NO: 75), EGKSSGSGSESKSTQ (SEQ ID NO: 76), GSTSGSGKSSEGKG (SEQ ID NO: 77), SSADDAKKDDAKKDDAKKDDAKKDG (SEQ ID NO: 78), EGKSSGSGSESKVD (SEQ ID NO: 79), ESGSVSSEELAFRSLD (SEQ ID NO: 80), EGKSSGSGSESKST (SEQ ID NO: 81), or EGKSSGSGSESKSTQ (SEQ ID NO: 82), or a functional variant thereof. Additional linkers include those described in, e.g., Whitlow and Filpula, Methods, Volume 2, Issue 2, April 1991, Pages 97-105, the content of which is incorporated herein by reference in its entirety.

In some embodiments, the linker sequence comprises the amino acid sequence GGGGSGGGGSGGGGS (SEQ ID NO: 10), or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 10. In certain embodiments, the nucleotide sequence that encodes the linker sequence comprises the nucleotide sequence that encodes the amino acid sequence of SEQ ID NO: 10, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 10. In certain embodiments, the nucleotide sequence that encodes the linker sequence comprises the nucleotide sequence set forth in SEQ ID NO: 11 or 12, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 11 or 12. In certain embodiments, the linker sequence comprises the amino acid sequence set forth in SEQ ID NO: 10. In certain embodiments, the nucleotide sequence that encodes the linker sequence comprises the nucleotide sequence set forth in SEQ ID NO: 11 or 12.

In some embodiments, the linker sequence comprises the amino acid sequence GGGGS (SEQ ID NO: 13), or a variant thereof having at least 80% sequence identity with SEQ ID NO: 13. In certain embodiments, the nucleotide sequence that encodes the linker sequence comprises the nucleotide sequence that encodes the amino acid sequence of SEQ ID NO: 13, or a variant thereof having at least 80% sequence identity with SEQ ID NO: 13. In certain embodiments, the nucleotide sequence that encodes the linker sequence comprises the nucleotide sequence set forth in SEQ ID NO: 14, or a nucleotide sequence having at least 60, at least 65, at least 70, at least 75, at least 80, at least 85, at least 90 sequence identity with SEQ ID NO: 14. In certain embodiments, the linker sequence comprises the amino acid sequence set forth in SEQ ID NO: 13. In certain embodiments, the nucleotide sequence that encodes the linker sequence comprises the nucleotide sequence set forth in SEQ ID NO: 14.

In certain embodiments, the antigen-binding moiety comprises a polypeptide or functional variant thereof that binds to a Col11A1 splice variant. In certain embodiments, the antigen-binding moiety is an antibody or an antibody fragment that binds to a Col11A1 splice variant. In certain embodiments, the antigen-binding moiety is a single chain variable fragment (scFv) that binds to a Col11A1 splice variant (anti-Col11A1 scFv). In some embodiments, the anti-Col11A1 scFv is derived from an mAb specific for the Col11A1 splice variant. In some embodiments, the Col11A1 splice variant contains at least exon 6 within the VAR sub-domain of the propeptide of Col11A1. In some embodiments, the Col11A1 splice variant contains exons 6 and 7 within the VAR sub-domain of the propeptide of Col11A1. In some embodiments, the Col11A1 splice variant contains exons 6, 7, 8 and 9 within the VAR sub-domain of the propeptide of Col11A1. In some embodiments, the binding moiety binds to exon 6 within the VAR sub-domain of the propeptide of Col11A1. In some embodiments, the anti-Col11A1 scFv is derived from a Col11A1 specific Mab 1E8.33 (1E8.33 scFv), or a functional variant thereof. The 1E8.33 antibody is an antibody specific for Col11A1 described in U.S. Pat. No. 9,702,879, which is herein incorporated by reference in its entirety for all purposes.

In some embodiments, 1E8.33 scFV comprises within the heavy chain variable region (VH) the following complementarity determining regions (CDRs): a heavy chain CDR1 (HCDR1) comprising the amino acid sequence shown in SEQ ID NO: 114 (GYSFTGYY); a heavy chain CDR2 (HCDR2) comprising the amino acid sequence shown in SEQ ID NO: 115 (INCYNGAT); and a heavy chain CDR3 (HCDR3) comprising the amino acid sequence shown in SEQ ID NO: 116 (AIWDYEFHVMDY).

In some embodiments, 1E8.33 scFV comprises within the light chain variable region (VL) the following complementarity determining regions (CDRs): a light chain CDR1 (LCDR1) comprising the amino acid sequence shown in SEQ ID NO: 117 (SSVNY); a light chain CDR2 (LCDR2) comprising the amino acid sequence YTS; and a light chain CDR3 (LCDR3) comprising the amino acid sequence shown in SEQ ID NO: 118 (QQFTSSPWT).

In some embodiments, 1E8.33 scFV comprises within the heavy chain variable region (VH) the following complementarity determining regions (CDRs): a heavy chain CDR1 (HCDR1) comprising the amino acid sequence shown in SEQ ID NO: 114 (GYSFTGYY); a heavy chain CDR2 (HCDR2) comprising the amino acid sequence shown in SEQ ID NO: 115 (INCYNGAT); and a heavy chain CDR3 (HCDR3) comprising the amino acid sequence shown in SEQ ID NO: 116 (AIWDYEFHVMDY); and comprises within the light chain variable region (VL) the following complementarity determining regions (CDRs): a light chain CDR1 (LCDR1) comprising the amino acid sequence shown in SEQ ID NO: 117 (SSVNY); a light chain CDR2 (LCDR2) comprising the amino acid sequence YTS; and a light chain CDR3 (LCDR3) comprising the amino acid sequence shown in SEQ ID NO: 118 (QQFTSSPWT).

In some embodiments, 1E8.33 scFV comprises a heavy chain variable domain (VH) comprising the amino acid sequence set forth in SEQ ID NO: 64, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 64. In certain embodiments, the nucleotide sequence that encodes the VH of 1E8.33 scFV comprises the nucleotide sequence that encodes the amino acid sequence of SEQ ID NO: 64, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 64. In certain embodiments, the nucleotide sequence that encodes the VH of 1E8.33 scFV comprises the nucleotide sequence set forth in SEQ ID NO: 65, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 65. In certain embodiments, the VH of 1E8.33 scFV comprises the amino acid sequence set forth in SEQ ID NO: 64. In certain embodiments, the nucleotide sequence that encodes the VH of 1E8.33 scFV comprises the nucleotide sequence set forth in SEQ ID NO: 65.

In some embodiments, 1E8.33 scFV comprises a light chain variable domain (VL) comprising the amino acid sequence set forth in SEQ ID NO: 68, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 68. In certain embodiments, the nucleotide sequence that encodes the VL of 1E8.33 scFV comprises the nucleotide sequence that encodes the amino acid sequence of SEQ ID NO: 68, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 68. In certain embodiments, the nucleotide sequence that encodes the VL of 1E8.33 scFV comprises the nucleotide sequence set forth in SEQ ID NO: 69, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 69. In certain embodiments, the VL of 1E8.33 scFV comprises the amino acid sequence set forth in SEQ ID NO: 68. In certain embodiments, the nucleotide sequence that encodes the VL of 1E8.33 scFV comprises the nucleotide sequence set forth in SEQ ID NO: 69.

In some embodiments, 1E8.33 scFV comprises the amino acid sequence set forth in SEQ ID NO: 4, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 4. In certain embodiments, the nucleotide sequence that encodes the 1E8.33 scFV comprises the nucleotide sequence that encodes the amino acid sequence of SEQ ID NO: 4, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 4. In certain embodiments, the nucleotide sequence that encodes the 1E8.33 scFV comprises the nucleotide sequence set forth in SEQ ID NO: 5, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 5. In certain embodiments, the 1E8.33 scFV comprises the amino acid sequence set forth in SEQ ID NO: 4. In certain embodiments, the nucleotide sequence that encodes the 1E8.33 scFV comprises the nucleotide sequence set forth in SEQ ID NO: 5.

In certain embodiments, the antigen-binding moiety comprises a polypeptide or functional variant thereof that binds to a tenascin C (TNC) splice variant. In certain embodiments, the antigen-binding moiety is an antibody or an antibody fragment that binds to a tenascin C splice variant. In certain embodiments, the antigen-binding moiety is a single chain variable fragment (scFv) that binds to a tenascin C splice variant (anti-TNC scFv). In some embodiments, the anti-TNC scFv is derived from an MAb specific for the TNC splice variant. In some embodiments, In some embodiments, the TNC splice variant contains at least the C domain of TNC (C.TNC). In some embodiments, the TNC splice variant contains exons A1, A2, A3, A4, B, AD2, AD1, C, and D of TNC. In some embodiments, the binding moiety binds to the C domain of TNC (C.TNC). In some embodiments, the anti-TNC scFv is derived from a C.TNC specific Mab G11 (G11 scFv), or a functional variant thereof. The G11 antibody is an antibody specific for C.TNC described in U.S. Pat. No. 7,968,685, which is herein incorporated by reference in its entirety for all purposes.

In some embodiments, G11 scFV comprises within the heavy chain variable region (VH) the following complementarity determining regions (CDRs): a heavy chain CDR1 (HCDR1) comprising the amino acid sequence shown in SEQ ID NO: 119 (GSRMG); a heavy chain CDR2 (HCDR2) comprising the amino acid sequence shown in SEQ ID NO: 120 (AINEEGGQTYYADSVK); and a heavy chain CDR3 (HCDR3) comprising the amino acid sequence shown in SEQ ID NO: 121 (HPPHRPFDY).

In some embodiments, G11 scFV comprises within the light chain variable region (VL) the following complementarity determining regions (CDRs): a light chain CDR1 (LCDR1) comprising the amino acid sequence shown in SEQ ID NO: 122 (QGDSLRLYYAS); a light chain CDR2 (LCDR2) comprising the amino acid sequence SEQ ID NO: 123 (GKNNRPS); and a light chain CDR3 (LCDR3) comprising the amino acid sequence shown in SEQ ID NO: 124 (NSSHGPRRPVV).

In some embodiments, G11 scFV comprises within the heavy chain variable region (VH) the following complementarity determining regions (CDRs): a heavy chain CDR1 (HCDR1) comprising the amino acid sequence shown in SEQ ID NO: 119 (GSRMG); a heavy chain CDR2 (HCDR2) comprising the amino acid sequence shown in SEQ ID NO: 120 (AINEEGGQTYYADSVK); and a heavy chain CDR3 (HCDR3) comprising the amino acid sequence shown in SEQ ID NO: 121 (HPPHRPFDY); and comprises within the light chain variable region (VL) the following complementarity determining regions (CDRs): a light chain CDR1 (LCDR1) comprising the amino acid sequence shown in SEQ ID NO: 122 (QGDSLRLYYAS); a light chain CDR2 (LCDR2) comprising the amino acid sequence SEQ ID NO: 123 (GKNNRPS); and a light chain CDR3 (LCDR3) comprising the amino acid sequence shown in SEQ ID NO: 124 (NSSHGPRRPVV).

In some embodiments, G11 scFV comprises a heavy chain variable domain (VH) comprising the amino acid sequence set forth in SEQ ID NO: 66, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 66. In certain embodiments, the nucleotide sequence that encodes the VH of G11 scFV comprises the nucleotide sequence that encodes the amino acid sequence of SEQ ID NO: 66, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 66. In certain embodiments, the nucleotide sequence that encodes the VH of G11 scFV comprises the nucleotide sequence set forth in SEQ ID NO: 67, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 67. In certain embodiments, the VH of G11 scFV comprises the amino acid sequence set forth in SEQ ID NO: 66. In certain embodiments, the nucleotide sequence that encodes the VH of G11 scFV comprises the nucleotide sequence set forth in SEQ ID NO: 67.

In some embodiments, G11 scFV comprises a light chain variable domain (VL) comprising the amino acid sequence set forth in SEQ ID NO: 70, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 70. In certain embodiments, the nucleotide sequence that encodes the VL of G11 scFV comprises the nucleotide sequence that encodes the amino acid sequence of SEQ ID NO: 70, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 70. In certain embodiments, the nucleotide sequence that encodes the VL of G11 scFV comprises the nucleotide sequence set forth in SEQ ID NO: 71, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 71. In certain embodiments, the VL of G11 scFV comprises the amino acid sequence set forth in SEQ ID NO: 70. In certain embodiments, the nucleotide sequence that encodes the VL of G11 scFV comprises the nucleotide sequence set forth in SEQ ID NO: 71.

In some embodiments, G11 scFV comprises the amino acid sequence set forth in SEQ ID NO: 6, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 6. In certain embodiments, the nucleotide sequence that encodes the G11 scFV comprises the nucleotide sequence that encodes the amino acid sequence of SEQ ID NO: 6, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 6. In certain embodiments, the nucleotide sequence that encodes the G11 scFV comprises the nucleotide sequence set forth in SEQ ID NO: 7, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 7. In certain embodiments, the G11 scFV comprises the amino acid sequence set forth in SEQ ID NO: 6. In certain embodiments, the nucleotide sequence that encodes the G11 scFV comprises the nucleotide sequence set forth in SEQ ID NO: 7.

In some embodiments, G11 scFV comprises the amino acid sequence set forth in SEQ ID NO: 8, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 8. In certain embodiments, the nucleotide sequence that encodes the G11 scFV comprises the nucleotide sequence that encodes the amino acid sequence of SEQ ID NO: 8, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 8. In certain embodiments, the nucleotide sequence that encodes the G11 scFV comprises the nucleotide sequence set forth in SEQ ID NO: 9, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 9. In certain embodiments, the G11 scFV comprises the amino acid sequence set forth in SEQ ID NO: 8. In certain embodiments, the nucleotide sequence that encodes the G11 scFV comprises the nucleotide sequence set forth in SEQ ID NO: 9.

### Leader Sequence

In certain aspects, the CAR of the present disclosure comprises a leader sequence. The leader sequence may be positioned amino-terminal to the extracellular target-binding domain. The leader sequence may be optionally cleaved from the antigen-binding moiety during cellular processing and localization of the CAR to the cellular membrane.

In some embodiments, the leader sequence may be derived from human immunoglobulin heavy chain variable region. In some embodiments, the leader sequence comprises the amino acid sequence set forth in SEQ ID NO: 1 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 1. In certain embodiments, the nucleotide sequence encoding the leader sequence comprises the nucleotide sequence that encodes the amino acid sequence of SEQ ID NO: 1, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 1. In certain embodiments, the nucleotide sequence encoding the leader sequence comprises the sequence set forth in SEQ ID NO: 2 or 3, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 2 or 3. In certain embodiments, the leader sequence comprises the amino acid sequence of SEQ ID NO: 1. In certain embodiments, the nucleotide sequence encoding the leader sequence comprises the nucleotide sequence set forth in SEQ ID NO: 2 or 3.

In some embodiments, the leader sequence may be derived from CD8α. In some embodiments, the leader sequence comprises the amino acid sequence set forth in SEQ ID NO: 98 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 98. In certain embodiments, the nucleotide sequence encoding the leader sequence comprises the nucleotide sequence that encodes the amino acid sequence of SEQ ID NO: 98, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 98. In certain embodiments, the nucleotide sequence encoding the leader sequence comprises the sequence set forth in SEQ ID NO: 99, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 99. In certain embodiments, the leader sequence comprises the amino acid sequence of SEQ ID NO: 98. In certain embodiments, the nucleotide sequence encoding the leader sequence comprises the nucleotide sequence set forth in SEQ ID NO: 99.

### Hinge Domain

In certain embodiments, the CAR further comprises a hinge domain between the extracellular antigen-binding domain and the transmembrane domain, wherein the antigen-binding moiety, linker, and the transmembrane domain are in frame with each other.

A hinge domain can comprise any oligo- or polypeptide that functions to link the antigen-binding moiety to the transmembrane domain. A hinge domain can be used to provide more flexibility and accessibility for the antigen-binding moiety. A hinge domain may comprise up to 300 amino acids, preferably 10 to 100 amino acids and most preferably 25 to 50 amino acids. A hinge domain may be derived from all or part of naturally occurring molecules, such as from all or part of the extracellular region of CD8, CD4 or CD28, or from all or part of an antibody constant region. Alternatively, the hinge domain may be a synthetic sequence that corresponds to a naturally occurring linker region sequence, or may be an entirely synthetic linker region sequence. Non-limiting examples of hinge domains which may be used in accordance with the disclosure include a part of human CD8a chain, partial extracellular domain of CD28, FcγRllla receptor, IgG, IgM, IgA, IgD, IgE, an Ig hinge, or functional fragment thereof. In some embodiments, additional linking amino acids are added to the linker region to ensure that the antigen-binding moiety is an optimal distance from the transmembrane domain. In some embodiments, when the hinge domain is derived from an Ig, the linker may be mutated to prevent Fc receptor binding.

In some embodiments, the hinge domain may be derived from CD8α, CD28, or an immunoglobulin (IgG). For example, the IgG hinge may be from IgG1, IgG2, IgG3, IgG4, IgM1, IgM2, IgA1, IgA2, IgD, IgE, or a chimera thereof.

In certain embodiments, the linker domain comprises an immunoglobulin IgG hinge or functional fragment thereof. In certain embodiments, the IgG hinge is from IgG1, IgG2, IgG3, IgG4, IgM1, IgM2, IgA1, IgA2, IgD, IgE, or a chimera thereof. In certain embodiments, the linker domain comprises the CH1, CH2, CH3 and/or hinge region of the immunoglobulin. In certain embodiments, the linker domain comprises the core hinge region of the immunoglobulin. The term “core hinge” can be used interchangeably with the term “short hinge” (a.k.a “SH”). Non-limiting examples of suitable linker domains are the core immunoglobulin hinge regions listed in Table 1 (see also Wypych et al., JBC 2008 283(23): 16194-16205, which is incorporated herein by reference in its entirety for all purposes). In certain embodiments, the linker domain is a fragment of the immunoglobulin hinge.

TABLE 1
Amino Acid Sequence of Short Hinge Regions of IgG Immunoglobulins

| IgG Subtype | Short Hinge Sequence | SEQ ID NO |
|---|---|---|
| IgG1 | EPKSCDKTHTCPPCP | SEQ ID NO: 83 |
| IgG1 | DLEPKSCDKTHTCPPCPDPK | SEQ ID NO: 15 |
| IgG2 | ERKCCVECPPCP | SEQ ID NO: 84 |
| IgG3 | ELKTPLGDTTHTCPRCP(EPKSCDTPPPCPRCP)₃ | SEQ ID NO: 85 |
| IgG4 | ESKYGPPCPSCP | SEQ ID NO: 86 |

In certain embodiments, the hinge domain comprises an IgG1 hinge, or a variant thereof. In certain embodiments, the hinge domain comprises the short hinge structure of IgG1, IgG2, IgG3, or IgG4 or a variant thereof. In certain embodiments, hinge domain comprises a short hinge region and comprises the amino acid sequence set forth in SEQ ID NO: 15, 83, 84, 85, or 86, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 15, 83, 84, 85, or 86. In certain embodiments, the nucleotide sequence encoding the hinge comprising the short hinge region comprises the nucleotide sequence that encodes the amino acid sequence of SEQ ID NO: 15, 83, 84, 85, or 86, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 15, 83, 84, 85, or 86. In certain embodiments, hinge domain comprises a short hinge region and comprises the amino acid sequence set forth in SEQ ID NO: 15, 83, 84, 85, or 86.

In certain embodiments, hinge domain comprises a short hinge region and comprises the amino acid sequence set forth in SEQ ID NO: 15, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 15. In certain embodiments, the nucleotide sequence encoding the hinge comprising the short hinge region comprises the nucleotide sequence that encodes the amino acid sequence of SEQ ID NO: 15, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 15. In certain embodiments, hinge domain comprises a short hinge region and comprises the amino acid sequence set forth in SEQ ID NO: 15. In certain embodiments, the nucleotide sequence encoding the hinge comprising the short hinge region comprises the nucleotide sequence of SEQ ID NO: 16, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 16. In certain embodiments, the nucleotide sequence encoding the hinge comprising the short hinge region comprises the nucleotide sequence of SEQ ID NO: 16.

In some embodiments, the hinge domain is derived from IgG4. In some embodiments, the hinge domain derived from IgG4 comprises the amino acid sequence set forth in SEQ ID NO: 17, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 17. In certain embodiments, the nucleotide sequence that encodes the IgG4 hinge domain comprises the nucleotide sequence that encodes the amino acid sequence of SEQ ID NO: 17, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 17. In certain embodiments, the nucleotide sequence that encodes the IgG4 hinge domain comprises the nucleotide sequence set forth in SEQ ID NO: 18, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 18. In certain embodiments, the IgG4 hinge domain comprises the amino acid sequence set forth in SEQ ID NO: 17. In certain embodiments, the nucleotide sequence that encodes the IgG4 hinge domain comprises the nucleotide sequence set forth in SEQ ID NO: 18.

In some embodiments, the hinge domain is derived from CD8a stalk or complete or partial sequences of the CD8a stalk, which are also called CD8a hinge. In some embodiments, the hinge domain derived from CD8a stalk comprises the amino acid sequence set forth in SEQ ID NO: 19, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 19. In certain embodiments, the nucleotide sequence that encodes the CD8a stalk hinge domain comprises the nucleotide sequence that encodes the amino acid sequence of SEQ ID NO: 19, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 19. In certain embodiments, the nucleotide sequence that encodes the CD8a stalk hinge domain comprises the nucleotide sequence set forth in SEQ ID NO: 20, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 20. In certain embodiments, the CD8a stalk hinge domain comprises the amino acid sequence set forth in SEQ ID NO: 19. In certain embodiments, the nucleotide sequence that encodes the CD8a stalk hinge domain comprises the nucleotide sequence set forth in SEQ ID NO: 20.

In some embodiments, the hinge domain is derived from CD28. In some embodiments, the hinge domain derived from CD28 hinge domain comprises the amino acid sequence set forth in SEQ ID NO: 100, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 100. In certain embodiments, the nucleotide sequence that encodes the CD28 hinge domain comprises the nucleotide sequence that encodes the amino acid sequence of SEQ ID NO: 100, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 100. In certain embodiments, the nucleotide sequence that encodes the CD28 hinge domain comprises the nucleotide sequence set forth in SEQ ID NO: 101, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 101. In certain embodiments, the CD28 hinge domain comprises the amino acid sequence set forth in SEQ ID NO: 100. In certain embodiments, the nucleotide sequence that encodes the CD28 hinge domain comprises the nucleotide sequence set forth in SEQ ID NO: 101.

In some embodiments, in addition to the sequences described above, the hinge domain can comprise additional linker amino acids to allow for extra flexibility and/or accessibility.

### Transmembrane Domain

In certain aspects, the CARs of the present disclosure comprise a transmembrane domain, fused in frame between the extracellular target-binding domain and the cytoplasmic domain.

The transmembrane domain may be derived from the protein contributing to the extracellular target-binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid-binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this disclosure may be derived from (i.e. comprise at least the transmembrane region(s) of) the α, β or ζ chain of the T-cell receptor, CD28, CD3 epsilon, CD45, CD4, CD5, CD8, CD8α, CD9, CD16, CD22, CD33, CD3γ, CD40, CD64, CD80, CD86, CD134, CD137, CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In certain embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid-binding of such domains to the transmembrane domains of the same or different surface membrane proteins to minimize interactions with other members of the receptor complex. In other cases, it will be desirable to employ the transmembrane domain of ζ, η or FcεR1γ and -β, MB1 (Igα), B29 or CD3-γ, ζ, or η, in order to retain physical association with other members of the receptor complex.

In certain embodiments, the transmembrane domain in the CAR of the disclosure is derived from the CD28 transmembrane domain. In certain embodiments, the CD28 transmembrane domain comprises the amino acid sequence set forth in SEQ ID NO: 21, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO:21. In certain embodiments, the nucleotide sequence that encodes the CD28 transmembrane domain comprises the nucleotide sequence that encodes the amino acid sequence of SEQ ID NO: 21, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO:21. In certain embodiments, the nucleotide sequence that encodes the CD28 transmembrane domain comprises the nucleotide sequence set forth in SEQ ID NO: 22, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 22. In certain embodiments, the CD28 transmembrane domain comprises the amino acid sequence set forth in SEQ ID NO: 21. In certain embodiments, the nucleotide sequence that encodes the CD28 transmembrane domain comprises the nucleotide sequence set forth in SEQ ID NO: 22.

In certain embodiments, the transmembrane domain in the CAR of the disclosure is derived from the CD8a transmembrane domain. In certain embodiments, the CD8a transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 23. In certain embodiments, the nucleotide sequence that encodes the CD8a transmembrane domain comprises the nucleotide sequence that encodes the amino acid sequence of SEQ ID NO: 23, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 23. In certain embodiments, the nucleotide sequence that encodes the CD8a transmembrane domain comprises the nucleotide sequence set forth in SEQ ID NO: 24, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 24. In certain embodiments, the CD8a transmembrane domain comprises the amino acid sequence set forth in SEQ ID NO: 23. In certain embodiments, the nucleotide sequence that encodes the CD8a transmembrane domain comprises the nucleotide sequence set forth in SEQ ID NO: 24.

In certain embodiments, the transmembrane domain in the CAR of the disclosure is derived from the CD3ζ transmembrane domain. In certain embodiments, the CD3ζ transmembrane domain comprises the amino acid sequence set forth in SEQ ID NO: 25, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 25. In certain embodiments, the nucleotide sequence that encodes the CD8a transmembrane domain comprises the nucleotide sequence that encodes the amino acid sequence of SEQ ID NO: 25, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 25. In certain embodiments, the nucleotide sequence that encodes the CD8a transmembrane domain comprises the nucleotide sequence set forth in SEQ ID NO: 26, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 26. In certain embodiments, the CD8a transmembrane domain comprises the amino acid sequence set forth in SEQ ID NO: 25. In certain embodiments, the nucleotide sequence that encodes the CD8a transmembrane domain comprises the nucleotide sequence set forth in SEQ ID NO: 26.

### Cytoplasmic Domain

In certain aspects, CARs of the present disclosure comprise a cytoplasmic domain, which comprises one or more costimulatory domains and one or more signaling domains. The cytoplasmic domain, which comprises one or more costimulatory domains and one or more signaling domains, is responsible for activation of at least one of the normal effector functions of the lymphocyte in which the CAR has been placed in. The term “effector function” refers to a specialized function of a cell. Effector function of a T-cell, for example, may be cytolytic activity or helper activity including the secretion of cytokines. Thus, the term “signaling domain” refers to the portion of a protein which transduces the effector function signal and directs the cell to perform a specialized function. While usually the entire signaling domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the signaling domain sufficient to transduce the effector function signal.

Non-limiting examples of signaling domains which can be used in the CARs of the present disclosure include, e.g., signaling domains derived from DAP10, DAP12, Fc epsilon receptor I γ chain (FCER1G), FcR β, CD3δ, CD3R, CD3γ, CD3ζ, CD5, CD22, CD226, CD66d, CD79A, and CD79B. In some embodiments, the CAR of the present disclosure comprises a signaling domain derived from CD3ζ.

In certain embodiments, the lymphocyte activation domain in the CAR of the disclosure is designed to comprise the signaling domain of CD3ζ. In certain embodiments, the CD3ζ signaling domain comprises the amino acid sequence set forth in SEQ ID NO: 29 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 29. In certain embodiments, the nucleotide sequence that encodes the CD3ζ signaling domain comprises the nucleotide sequence that encodes the amino acid sequence of SEQ ID NO: 29, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 29. In certain embodiments, the nucleotide sequence that encodes the CD3ζ signaling domain comprises the nucleotide sequence set forth in SEQ ID NO: 30, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 30. In certain embodiments, the CD3ζ signaling domain comprises the amino acid sequence set forth in SEQ ID NO: 29. In certain embodiments, the nucleotide sequence that encodes the CD3ζ signaling domain comprises the nucleotide sequence set forth in SEQ ID NO: 30.

Non-limiting examples of costimulatory domains which can be used in the CARs of the present disclosure include, those derived from 4-1BB (CD137), CD28, CD40, ICOS, CD134 (OX-40), BTLA, CD27, CD30, GITR, CD226, CD79A, HVEM, MyD88, IL-2Rβ, or the STAT3-binding YXXQ. In some embodiments, the CAR of the present disclosure comprises one costimulatory domain. In some embodiments, the CAR of the present disclosure comprises a costimulatory domain derived from CD28.

In some embodiments, the CAR of the present disclosure comprises two or more costimulatory domains. In certain embodiments, the CAR of the present disclosure comprises two, three, four, five, six or more costimulatory domains. For example, the CAR of the present disclosure may comprise a costimulatory domain derived from 4-1BB and a costimulatory domain derived from CD28.

In certain embodiments, the CARs of the present disclosure comprise a cytoplasmic domain, which comprises a signaling domain, a MyD88 polypeptide or functional fragment thereof, and a CD40 cytoplasmic polypeptide region or a functional fragment thereof. In certain embodiments, the CAR lacks the CD40 transmembrane and/or CD40 extracellular domains. In certain embodiments, the CAR includes the CD40 transmembrane domain. In certain embodiments, the CAR includes the CD40 transmembrane domain and a portion of the CD40 extracellular domain, wherein the CD40 extracellular domain does not interact with natural or synthetic ligands of CD40.

In certain embodiments, the signaling domain is separated from the MyD88 polypeptide or functional fragment thereof and/or the CD40 cytoplasmic polypeptide region or a functional fragment thereof. In certain embodiments, the lymphocyte activation domain is separated from the MyD88 polypeptide or functional fragment thereof and/or the CD40 cytoplasmic polypeptide region or a functional fragment thereof by at least 1, 2, 3, 4, 5, 6, 7, 8, 9, 10, 11, 12, 13, 14, or 15 amino acids.

In some embodiments, the signaling domain(s) and costimulatory domain(s) can be in any order. In some embodiments, the signaling domain is upstream of the costimulatory domains. In some embodiments, the signaling domain is downstream from the costimulatory domains. In the cases where two or more costimulatory domains are included, the order of the costimulatory domains could be switched.

In some embodiments, the costimulatory domain derived from CD28 comprises the amino acid sequence set forth in SEQ ID NO: 27, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 27. In certain embodiments, the nucleotide sequence that encodes the CD28 costimulatory domain comprises the nucleotide sequence that encodes the amino acid sequence of SEQ ID NO: 27, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 27. In certain embodiments, the nucleotide sequence that encodes the CD28 costimulatory domain comprises the nucleotide sequence set forth in SEQ ID NO: 28, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 28. In certain embodiments, the CD28 costimulatory domain comprises the amino acid sequence set forth in SEQ ID NO: 27. In certain embodiments, the nucleotide sequence that encodes the CD28 costimulatory domain comprises the nucleotide sequence set forth in SEQ ID NO: 28.

In some embodiments, the costimulatory domain derived from 4-1BB comprises the amino acid sequence set forth in SEQ ID NO: 102, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 102. In certain embodiments, the nucleotide sequence that encodes the 4-1BB costimulatory domain comprises the nucleotide sequence that encodes the amino acid sequence of SEQ ID NO: 102, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 102. In certain embodiments, the nucleotide sequence that encodes the 4-1BB costimulatory domain comprises the nucleotide sequence set forth in SEQ ID NO: 103, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 103. In certain embodiments, the 4-1BB costimulatory domain comprises the amino acid sequence set forth in SEQ ID NO: 102. In certain embodiments, the nucleotide sequence that encodes the 4-1BB costimulatory domain comprises the nucleotide sequence set forth in SEQ ID NO: 103.

In some embodiments, the costimulatory domain derived from OX40 comprises the amino acid sequence set forth in SEQ ID NO: 104, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 104. In certain embodiments, the nucleotide sequence that encodes the OX40 costimulatory domain comprises the nucleotide sequence that encodes the amino acid sequence of SEQ ID NO: 104, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 104. In certain embodiments, the nucleotide sequence that encodes the OX40 costimulatory domain comprises the nucleotide sequence set forth in SEQ ID NO: 105, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 105. In certain embodiments, the OX40 costimulatory domain comprises the amino acid sequence set forth in SEQ ID NO: 104. In certain embodiments, the nucleotide sequence that encodes the OX40 costimulatory domain comprises the nucleotide sequence set forth in SEQ ID NO: 105.

In certain embodiments, the MyD88 polypeptide or functional fragment thereof in the CAR of the disclosure is designed to comprise the co-stimulatory domain of MyD88, or variant thereof. In certain embodiments, the MyD88 functional fragment comprises the amino acid sequence set forth in SEQ ID NO: 106, 108, or 110, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 106, 108, or 110. In certain embodiments, the nucleotide sequence encoding the MyD88 functional fragment comprises the nucleotide sequence that encodes the amino acid sequence of SEQ ID NO: 106, 108, or 110, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 106, 108, or 110. In certain embodiments, the nucleotide sequence encoding the MyD88 functional fragment comprises the nucleotide sequence set forth in SEQ ID NO: 107, 109, or 111, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 107, 109, or 111. In certain embodiments, the MyD88 functional fragment comprises the amino acid sequence set forth in SEQ ID NO: 106, 108, or 110. In certain embodiments, the nucleotide sequence that encodes the MyD88 functional fragment comprises the nucleotide sequence set forth in SEQ ID NO: 107, 109, or 111.

In certain embodiments, the CD40 polypeptide or functional fragment thereof in the CAR of the disclosure is designed to comprise the CD40 cytoplasmic polypeptide region. In certain embodiments, the CD40 cytoplasmic polypeptide region comprises the amino acid sequence set forth in SEQ ID NO: 112 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 112. In certain embodiments, the nucleotide sequence encoding the CD40 cytoplasmic polypeptide region comprises the nucleotide sequence that encodes the amino acid sequence of SEQ ID NO: 112, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 112. In certain embodiments, the nucleotide sequence encoding the CD40 cytoplasmic polypeptide region comprises the nucleotide sequence set forth in SEQ ID NO: 113, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 113. In certain embodiments, the CD40 cytoplasmic polypeptide region comprises the amino acid sequence of SEQ ID NO: 112. In certain embodiments, the nucleotide sequence encoding the CD40 cytoplasmic polypeptide region comprises the nucleotide sequence set forth in SEQ ID NO: 113.

### Additional Genes

In addition to the CAR construct, the CAR may further comprise at least one additional gene that encodes an additional peptide. Examples of additional genes can include a transduced host cell selection marker, an in vivo tracking marker, a cytokine, a suicide gene, or some other functional gene. In certain embodiments, the functional additional gene can induce the expression of another molecule. In certain embodiments, the functional additional gene can increase the safety of the CAR. For example, the CAR construct may comprise an additional gene which is truncated CD19 (tCD19). The tCD19 can be used as a tag. Expression of tCD19 may also help determine transduction efficiency.

Other examples of additional genes include genes that encode polypeptides with a biological function; examples include, but are not limited to, cytokines, chimeric cytokine receptors, dominant negative receptors, safety switches (CD20, truncated EGFR or HER2, inducible caspase 9 molecules). As another example, the CAR construct may comprise an additional gene which is a synNotch receptor. Once activated, the synNotch receptor can induce the expression of a target gene (e.g., a second CAR and/or bispecific molecule).

In certain embodiments, the CAR comprises at least one additional gene (i.e., a second gene). In certain embodiments, the CAR comprises one second gene. In other embodiments, the CAR comprises two additional genes (i.e., a third gene). In yet another embodiment, the CAR comprises three additional genes (i.e., a fourth gene). In certain embodiments, the additional genes are separated from each other and the CAR construct. For example, they may be separated by 2A sequences and/or an internal ribosomal entry sites (IRES). In certain examples, the CAR can be at any position of the polynucleotide chain (for example construct A: CAR, second gene, third gene, fourth gene; construct B: second gene, CAR, third gene, fourth gene; etc.)

Non-limiting examples of classes of additional genes that can be used to increase the effector function of CAR containing host cells, include (a) secretable cytokines (e.g., but not limited to, IL-7, IL-12, IL-15, IL-18), (b) membrane bound cytokines (e.g., but not limited to, IL-15), (c) chimeric cytokine receptors (e.g., but not limited to, IL-2/IL-7, IL-4/IL-7), (d) constitutive active cytokine receptors (e.g., but not limited to, C7R), (e) dominant negative receptors (DNR; e.g., but not limited to TGFRII DNR), (f) ligands of costimulatory molecules (e.g., but not limited to, CD80, 4-1BBL), (g) nuclear factor of activated T-cells (NFATs) (e.g., NFATc1, NFATc2, NFATc3, NFATc4, and NFAT5), (h) antibodies, including fragments thereof and bispecific antibodies (e.g., but not limited to, bispecific T-cell engagers (BiTEs)), or (i) a second CAR.

In some embodiments, the additional gene sequence may be derived from tCD19. In some embodiments, the tCD19 sequence comprises the amino acid sequence set forth in SEQ ID NO: 33 or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 33. In certain embodiments, the nucleotide sequence encoding the tCD19 sequence comprises the nucleotide sequence that encodes the amino acid sequence of SEQ ID NO: 33, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 33. In certain embodiments, the nucleotide sequence encoding the tCD19 sequence comprises the sequence set forth in SEQ ID NO: 34 or 35, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 34 or 35. In certain embodiments, the tCD19 sequence comprises the amino acid sequence of SEQ ID NO: 33. In certain embodiments, the nucleotide sequence encoding the tCD19 sequence comprises the nucleotide sequence set forth in SEQ ID NO: 34 or 35.

In certain embodiments, the additional gene may be regulated by an NFAT dependent-promoter. Activation of the T-cell or other lymphocyte leads to activation of the transcription factor NFAT resulting in the induction of the expression of the protein encoded by the gene linked with the NFAT dependent promoter. One or more members of the NFAT family (i.e., NFATc1, NFATc2, NFATc3, NFATc4, and NFAT5) is expressed in most cells of the immune system. NFAT-dependent promoters and enhancers tend to have three to five NFAT binding sites

In certain embodiments, the functional additional gene can be a suicide gene. A suicide gene is a recombinant gene that will cause the host cell that the gene is expressed in to undergo programmed cell death or antibody mediated clearance at a desired time. Suicide genes can function to increase the safety of the CAR. In another embodiment, the additional gene is an inducible suicide gene. Non-limiting examples of suicide genes include i) molecules that are expressed on the cell surface and can be targeted with a clinical grade monoclonal antibody including CD20, EGFR or a fragment thereof, HER2 or a fragment thereof, and ii) inducible suicide genes (e.g., but not limited to inducible caspase 9 (see Straathof et al. (2005) Blood. 105(11): 4247-4254; US Publ. No. 2011/0286980, each of which are incorporated herein by reference in their entirety for all purposes)).

In certain aspects, CARs of the present disclosure may be regulated by a safety switch. As used herein, the term “safety switch” refers to any mechanism that is capable of removing or inhibiting the effect of a CAR from a system (e.g., a culture or a subject). Safety switches can function to increase the safety of the CAR.

The function of the safety switch may be inducible. Non-limiting examples of safety switches include (a) molecules that are expressed on the cell surface and can be targeted with a clinical grade monoclonal antibody including CD20, EGFR or a fragment thereof, HER2 or a fragment thereof, and (b) inducible suicide genes (e.g., but not limited to herpes simplex virus thymidine kinase (HSV-TK) and inducible caspase 9 (see Straathof et al. (2005) Blood. 105(11): 4247-4254; US Publ. No. 2011/0286980, each of which are incorporated herein by reference in their entirety for all purposes).

In some embodiments, the safety switch is a CD20 polypeptide. Expression of human CD20 on the cell surface presents an attractive strategy for a safety switch. The inventors and others have shown that cells that express CD20 can be rapidly eliminated with the FDA approved monoclonal antibody rituximab through complement-mediated cytotoxicity and antibody-dependent cell-mediated cytotoxicity (see e.g., Griffioen, M., et al. Haematologica 94, 1316-1320 (2009), which is incorporated herein by reference in its entirety for all purposes). Rituximab is an anti-CD20 monoclonal antibody that has been FDA approved for Chronic Lymphocytic Leukemia (CLL) and Non-Hodgkin's Lymphoma (NHL), among others (Storz, U. MAbs 6, 820-837 (2014), which is incorporated herein by reference in its entirety for all purposes). The CD20 safety switch is non-immunogenic and can function as a reporter/selection marker in addition to a safety switch (Bonifant, C. L., et al. Mol Ther 24, 1615-1626 (2016); van Loenen, M. M., et al. Gene Ther 20, 861-867 (2013); each of which is incorporated herein by reference in its entirety for all purposes).

In some embodiments, the sequence encoding an additional gene is operably linked to the sequence encoding CAR via a sequence encoding a self-cleaving peptide and/or an Internal Ribosome Entry Site (IRES) as disclosed herein.

Non-limiting examples of self-cleaving peptide sequences includes Thoseaasigna virus 2A (T2A; AEGRGSLLTCGDVEENPGP, SEQ ID NO: 87, EGRGSLLTCGDVEENPGP, SEQ ID NO: 31, or GSGEGRGSLLTCGDVEENPGP, SEQ ID NO: 88); the foot and mouth disease virus (FMDV) 2A sequence (F2A; GSGSRVTELLYRMKRAETYCPRPLLAIIIPTEARHKQKIVAPVKQLLNFDLLKLAGDVES NPGP, SEQ ID NO: 89), Sponge (Amphimedon queenslandica) 2A sequence (LLCFLLLLLSGDVELNPGP, SEQ ID NO: 90; or HHFMFLLLLLAGDIELNPGP, SEQ ID NO: 91); acorn worm 2A sequence (Saccoglossus kowalevskii) (WFLVLLSFILSGDIEVNPGP, SEQ ID NO: 92); amphioxus (Branchiostoma floridae) 2A sequence (KNCAMYMLLLSGDVETNPGP, SEQ ID NO: 93; or MVISQLMLKLAGDVEENPGP, SEQ ID NO: 94); porcine teschovirus-1 2A sequence (P2A; GSGATNFSLLKQAGDVEENPGP, SEQ ID NO: 95); and equine rhinitis A virus 2A sequence (E2A; GSGQCTNYALLKLAGDVESNPGP, SEQ ID NO: 96). In some embodiments, the separation sequence is a naturally occurring or synthetic sequence. In certain embodiments, the separation sequence includes the 2A consensus sequence D-X-E-X-NPGP (SEQ ID NO: 97), in which X is any amino acid residue.

Alternatively, an Internal Ribosome Entry Site (IRES) may be used to link the CAR and the additional gene. IRES is an RNA element that allows for translation initiation in a cap-independent manner. IRES can link two coding sequences in one bicistronic vector and allow the translation of both proteins in cells.

In some embodiments, the self-cleaving 2A peptide is a T2A peptide and comprises the amino acid sequence set forth in SEQ ID NO: 31. In some embodiments, the sequence encoding the T2A peptide comprises the nucleotide sequence SEQ ID NO: 32.

In certain embodiments, the host cells can be genetically modified to express not only CARs as disclosed herein but to also express fusion protein with signaling activity (e.g., costimulation, T-cell activation). These fusion proteins can improve host cell activation and/or responsiveness. In certain embodiments, the fusion protein can enhance the host cell's response to the target antigen. In certain embodiments, the fusion protein can impart resistance to suppression signals.

In certain embodiments, fusion proteins can comprise portions of CD4, CD8α, CD28, portions of a T-cell receptor, or an antigen-binding moiety (e.g., scFv) linked to a MyD88, CD40, and/or other signaling molecules.

In certain embodiments, the fusion protein comprises an extracellular target-binding domain (as disclosed above), a transmembrane domain (as described above) and a cytoplasmic domain, wherein the cytoplasmic domain comprises at least one co-stimulatory protein (as described above). In certain embodiments, the co-stimulatory fusion protein does not comprise a lymphocyte activation domain (e.g., CD3ζ). In certain embodiments, the at least one co-stimulatory protein can be a MyD88 polypeptide or functional fragment thereof, and/or a CD40 cytoplasmic polypeptide region or a functional fragment thereof.

In certain embodiments, the fusion protein comprises an extracellular domain (such as, but not limited to CD19, CD34), a transmembrane domain (as described above) and a cytoplasmic domain, wherein the cytoplasmic domain comprises at least one co-stimulatory protein (as described above). In certain embodiments, the fusion protein does not comprise a lymphocyte activation domain (e.g., CD3ζ). In certain embodiments, the at least one portion of the fusion protein can be a MyD88 polypeptide or functional fragment thereof, and/or a CD40 cytoplasmic polypeptide region or a functional fragment thereof.

Non-limiting examples of fusion proteins include, but are not limited to, the constructs in the publication of WO2019222579 and WO2016073875, which are incorporated herein by reference in its entirety for all purposes.

In certain embodiments, the fusion proteins are introduced into the host cell on a separate vector from the CAR. In certain embodiments, the fusion proteins are introduced into the host cell on the same vector as the CAR. In certain embodiments, the fusion proteins are introduced into the host cell on the same vector as the CAR but separated by a separation sequence such as 2A.

### Non-Limited Examples of CARs

In certain embodiments, an anti-Col11A1 CAR of the disclosure comprises an extracellular binding domain comprising the amino acid sequence of SEQ ID NO: 36, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 36. In certain embodiments, the extracellular binding domain of an anti-Col11A1 CAR of the disclosure is encoded by a nucleotide sequence that encodes the amino acid sequence of SEQ ID NO: 36, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 36. In certain embodiments, the nucleotide sequence that encodes the extracellular binding domain of an anti-Col11A1 CAR of the disclosure comprises the nucleotide sequence set forth in SEQ ID NO: 37, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 37. In certain embodiments, an anti-Col11A1 CAR of the disclosure comprises an extracellular binding domain comprising the amino acid sequence set forth in SEQ ID NO: 36. In certain embodiments, the nucleotide sequence that encodes the extracellular binding domain of an anti-Col11A1 CAR of the disclosure comprises the nucleotide sequence set forth in SEQ ID NO: 37.

In certain embodiments, an anti-Col11A1 CAR of the disclosure comprises a cytoplasmic domain comprising the amino acid sequence of SEQ ID NO: 48, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 48. In certain embodiments, the cytoplasmic domain of an anti-Col11A1 CAR of the disclosure is encoded by a nucleotide sequence that encodes the amino acid sequence of SEQ ID NO: 48, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 48. In certain embodiments, the nucleotide sequence that encodes the cytoplasmic domain of an anti-Col11A1 CAR of the disclosure comprises the nucleotide sequence set forth in SEQ ID NO: 49, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 49. In certain embodiments, an anti-Col11A1 CAR of the disclosure comprises a cytoplasmic domain comprising the amino acid sequence set forth in SEQ ID NO: 48. In certain embodiments, the nucleotide sequence that encodes the cytoplasmic domain of an anti-Col11A1 CAR of the disclosure comprises the nucleotide sequence set forth in SEQ ID NO: 49.

In certain embodiments, an anti-Col11A1 CAR of the disclosure comprises the amino acid sequence of SEQ ID NO: 52, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 52. In certain embodiments, an anti-Col11A1 CAR of the disclosure is encoded by a nucleotide sequence that encodes the amino acid sequence of SEQ ID NO: 52, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 52. In certain embodiments, the nucleotide sequence that encodes an anti-Col11A1 CAR of the disclosure comprises the nucleotide sequence set forth in SEQ ID NO: 53, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 53. In certain embodiments, an anti-Col11A1 CAR of the disclosure comprises an amino acid sequence set forth in SEQ ID NO: 52. In certain embodiments, the nucleotide sequence that encodes an anti-Col11A1 CAR of the disclosure comprises the nucleotide sequence set forth in SEQ ID NO: 53.

In certain embodiments, an anti-C.TNC CAR of the disclosure comprises an extracellular binding domain comprising the amino acid sequence of SEQ ID NO: 38, 40, 42, 44, or 46, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 38, 40, 42, 44, or 46. In certain embodiments, the extracellular binding domain of an anti-C.TNC CAR of the disclosure is encoded by a nucleotide sequence that encodes the amino acid sequence of SEQ ID NO: 38, 40, 42, 44, or 46, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 38, 40, 42, 44, or 46. In certain embodiments, the nucleotide sequence that encodes the extracellular binding domain of an anti-C.TNC CAR of the disclosure comprises the nucleotide sequence set forth in SEQ ID NO: 39, 41, 43, 45, or 47, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 39, 41, 43, 45, or 47. In certain embodiments, an anti-C.TNC CAR of the disclosure comprises an extracellular binding domain comprising the amino acid sequence set forth in SEQ ID NO: 38, 40, 42, 44, or 46. In certain embodiments, the nucleotide sequence that encodes the extracellular binding domain of an anti-C.TNC CAR of the disclosure comprises the nucleotide sequence set forth in SEQ ID NO: 39, 41, 43, 45, or 47.

In certain embodiments, an anti-C.TNC CAR of the disclosure comprises a cytoplasmic domain comprising the amino acid sequence of SEQ ID NO: 48 or 50, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 48 or 50. In certain embodiments, the cytoplasmic domain of an anti-C.TNC CAR of the disclosure is encoded by a nucleotide sequence that encodes the amino acid sequence of SEQ ID NO: 48 or 50, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 48 or 50. In certain embodiments, the nucleotide sequence that encodes the cytoplasmic domain of an anti-C.TNC CAR of the disclosure comprises the nucleotide sequence set forth in SEQ ID NO: 49 or 51, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 49 or 51. In certain embodiments, an anti-C.TNC CAR of the disclosure comprises a cytoplasmic domain comprising the amino acid sequence set forth in SEQ ID NO: 48 or 50. In certain embodiments, the nucleotide sequence that encodes the cytoplasmic domain of an anti-C.TNC CAR of the disclosure comprises the nucleotide sequence set forth in SEQ ID NO: 49 or 51.

In certain embodiments, an anti-C.TNC CAR of the disclosure comprises the amino acid sequence of SEQ ID NO: 54, 56, 58, 60, 62, or 125, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 54, 56, 58, 60, 62, or 125. In certain embodiments, an anti-C.TNC CAR of the disclosure is encoded by a nucleotide sequence that encodes the amino acid sequence of SEQ ID NO: 54, 56, 58, 60, 62, or 125, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 54, 56, 58, 60, 62, or 125. In certain embodiments, the nucleotide sequence that encodes an anti-C.TNC CAR of the disclosure comprises the nucleotide sequence set forth in SEQ ID NO: 55, 57, 59, 61, 63, or 126, or a nucleotide sequence having at least 50, at least 55, at least 60, at least 65, at least 70, at least 75, at least 80, at least 85, at least 90, at least 95, at least 96, at least 97, at least 98 or at least 99%, sequence identity with SEQ ID NO: 55, 57, 59, 61, 63, or 126. In certain embodiments, an anti-C.TNC CAR of the disclosure comprises an amino acid sequence set forth in SEQ ID NO: 54, 56, 58, 60, 62, or 125. In certain embodiments, the nucleotide sequence that encodes an anti-C.TNC CAR of the disclosure comprises the nucleotide sequence set forth in SEQ ID NO: 55, 57, 59, 61, 63, or 126.

In certain embodiments, the CAR can be encoded by one polypeptide chain. In certain embodiments, the CAR can be encoded by two polypeptide chains. For example, the first polypeptide chain can encode an extracellular target-binding domain comprising an antigen-binding moiety, a transmembrane domain, and a short cytoplasmic tail, and the second polypeptide chain can encode a short extracellular domain, a transmembrane domain, and a cytoplasmic domain comprising a signaling domain, a MyD88 polypeptide or functional fragment thereof, and a CD40 cytoplasmic polypeptide region or a functional fragment thereof. In certain embodiments, both polypeptides can interact via their respective transmembrane domain.

In various embodiments, the polynucleotide encoding a CAR is a DNA molecule. In various embodiments, the polynucleotide encoding a CAR is an RNA molecule.

In one aspect, the present disclosure provides CAR polypeptides encoded by a polynucleotide described above.

### Vectors

The present disclosure provides recombinant vectors comprising a polynucleotide encoding a CAR comprising polynucleotides encoding the proteins disclosed above. In certain embodiments, the polynucleotide is operatively linked to at least one regulatory element for expression of the chimeric antigen receptor.

In certain embodiments, recombinant vectors of the disclosure comprise the nucleotide sequence of SEQ ID NO: 53, 55, 57, 59, 61, 63, or 126, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 53, 55, 57, 59, 61, 63, or 126. In certain embodiments, recombinant vectors comprise a nucleotide sequence that encodes the amino acid sequence of SEQ ID NO: 52, 54, 56, 58, 60, 62, or 125, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 52, 54, 56, 58, 60, 62, or 125.

In certain embodiments, the recombinant vector comprises a polynucleotide encoding a CAR, wherein the polynucleotide is operatively linked to at least one additional gene. In some embodiments, the additional gene is a tCD19.

In certain embodiments, the vector is a viral vector. In certain embodiments, the viral vector can be, but is not limited to, a retroviral vector, an adenoviral vector, an adeno-associated virus vector, an alphaviral vector, a herpes virus vector, and a vaccinia virus vector. In some embodiments, the viral vector is a lentiviral vector.

In some embodiments, the vector is a non-viral vector. The viral vector may be a plasmid or a transposon (such as a PiggyBac- or a Sleeping Beauty transposon).

In certain embodiments, the polynucleotide encoding the CAR is operably linked to at least a regulatory element. The regulatory element can be capable of mediating expression of the CAR in the host cell. Regulatory elements include, but are not limited to, promoters, enhancers, initiation sites, polyadenylation (polyA) tails, IRES elements, response elements, and termination signals. In certain embodiments, the regulatory element regulates CAR expression. In certain embodiments, the regulatory element increased the expression of the CAR. In certain embodiments, the regulatory element increased the expression of the CAR once the host cell is activated. In certain embodiments, the regulatory element decreases expression of the CAR. In certain embodiments, the regulatory element decreases expression of the CAR once the host cell is activated.

### CAR-Modified Host Cells

In one aspect, the present disclosure provides an isolated host cell comprising a polynucleotide or a recombinant vector described herein. In one aspect, the present disclosure provides an isolated host cell comprising a CAR described herein. In some embodiments, the CAR targets a procollagen 11A1 (Col11A1) splice variant. In some embodiments, the CAR targets a tenascin C (TNC) splice variant.

In a further aspect, the present disclosure provides an isolated host cell comprising two or more polynucleotides or recombinant vectors described herein. In a further aspect, the present disclosure provides an isolated host cell comprising two or more CARs described herein. For example, an isolated host cell may comprise a CAR targeting a procollagen 11A1 (Col11A1) splice variant and a CAR targeting a tenascin C (TNC) splice variant.

In various embodiments, the host cell is an immune cell. The immune cell may be a T-cell, a natural killer (NK) cell or a macrophage.

In various embodiments, the host cell is a T-cell. T-cells may include, but are not limited to, thymocytes, naïve T lymphocytes, immature T lymphocytes, mature T lymphocytes, resting T lymphocytes, or activated T lymphocytes. A T-cell can be a T helper (Th) cell, for example a T helper 1 (Th1) or a T helper 2 (Th2) cell. The T-cell can be a helper T-cell (HTL; CD4+ T-cell) CD4+ T-cell, a cytotoxic T-cell (CTL; CD8+ T-cell), a tumor infiltrating cytotoxic T-cell (TIL; CD8+ T-cell), CD4+ CD8+ T-cell, or any other subset of T-cells. Other illustrative populations of T-cells suitable for use in particular embodiments include naïve T-cells memory T-cells, and NKT cells.

In some embodiments, the T-cell is selected from a CD8+ T-cell, a CD4+ T-cell, a cytotoxic T-cell, an ap T-cell receptor (TCR) T-cell, a natural killer T (NKT) cell, a γδ T-cell, a memory T-cell, a T-helper cell, and a regulatory T-cell (Treg).

In various embodiments, the host cell is a NK cell. NK cell refers to a differentiated lymphocyte with a CD3− CD16+, CD3− CD56+, CD16+ CD56+ and/or CD57+ TCR− phenotype.

In various embodiments, the host cell has been activated and/or expanded ex vivo.

In various embodiments, the host cell is an allogeneic cell. In various embodiments, the host cell is an autologous cell.

In some embodiments, the host cell is isolated from a subject having a tumor. In some embodiments, the tumor can be found within, but not limited to, breast tissue, prostate tissue, bladder tissue, oral and/or dental tissue, head and/or neck tissue, colorectal tissue, lung tissue, brain tissue, skin, lymph nodes, and bone. In some embodiments, the tumor is a cancer. In some embodiments, the cancer can be, but not limited to, breast cancer, prostate cancer, bladder cancer, oral squamous cell carcinoma, head and/or neck squamous cell carcinoma, colorectal cancer, lung cancer, brain tumors, melanoma, bone, pediatric solid tumors and brain tumors, and/or lymphoma.

In certain embodiments, the host cell is isolated from a subject having a tumor, wherein one or more cells of the tumor cells express a procollagen 11A1 (Col11A1) splice variant. Non-limiting examples of tumor cells that express a procollagen 11A1 (Col11A1) splice variant include acute lymphoblastic leukemia, acute myeloid leukemia, adult solid tumors and brain tumors, adrenal gland tumors, anal cancer, bile duct cancer, bladder cancer, blood cancers, bone cancer, bowel cancer, brain tumors, breast cancer, cancer of unknown primary, cancer spread to bone, cancer spread to brain, cancer spread to liver, cancer spread to lung, carcinoid, cervical cancer, children's cancers, colorectal cancer, chronic lymphocytic leukemia, chronic myeloid leukemia, colorectal cancer, ear cancer, endometrial cancer, eye cancer, follicular dendritic cell sarcoma, gallbladder cancer, gastric cancer, gastro esophageal junction cancers, germ cell tumors, gestational trophoblastic disease, glioma, glioblastoma, gynecological cancer, hairy cell leukemia, head and neck squamous cell carcinoma, high grade gliomas, Hodgkin lymphoma, Kaposi's sarcoma, kidney cancer, large bowel and rectal neuroendocrine tumors, laryngeal cancer, leukemia, Linitis plastica of the stomach, liver cancer, low grade gliomas, lung cancer, lung neuroendocrine tumors (NETs), lymphoma, malignant schwannoma, mediastinal germ cell tumors, melanoma, men's cancer, merkel cell skin cancer, mesothelioma, molar pregnancy, mouth and oropharyngeal cancer, myeloma, nasal and paranasal sinus cancer, nasopharyngeal cancer, neuroblastoma, neuroendocrine tumors, neuroendocrine tumors of the pancreas, non-Hodgkin lymphoma, non-Hodgkin lymphoma in children, esophageal cancer, oral squamous cell carcinoma, ovarian cancer, pancreatic cancer, pediatric solid tumors and brain tumors, penile cancer, persistent trophoblastic disease and choriocarcinoma, pheochromocytoma, prostate cancer, pseudomyxoma peritonei, rare cancers, rectal cancer, renal cancer, retinoblastoma, salivary gland cancer, secondary cancer, signet cell cancer, skin cancer, small bowel cancer, small bowel neuroendocrine tumors, soft tissue sarcoma, stomach cancer, stomach neuroendocrine tumors, testis cancer, thymus gland tumors, thyroid cancer, tongue cancer, tonsil cancer, tumors of the adrenal gland, unknown primary cancer, urothelial, uterine cancer, vaginal cancer, vulval cancer, Wilms' tumor, and womb cancer.

In certain embodiments, the host cell is isolated from a subject having a tumor, wherein one or more cells of the tumor cells express a C domain of tenascin C (C.TNC) splice variant. Non-limiting examples of tumor cells that express the C domain of tenascin C (C.TNC) splice variant include glioblastoma, high grade gliomas, low grade gliomas, head and neck cancers, liver cancers, lung cancers, melanoma, ovarian cancer, pancreatic cancer, prostate cancer, renal cancer, urothelial cancer, carcinoid, cervical cancers, colorectal cancer, endometrial cancer, lymphoma, skin cancer, stomach cancer, testis cancer, thyroid cancer and urothelial cancer.

In some embodiments, the host cell is derived from a blood, marrow, tissue, or a tumor sample.

In one aspect, the present disclosure provides a method of generating an isolated host cell described herein. The method includes genetically modifying the host cell with a polynucleotide encoding a CAR and optionally an additional gene (e.g., tCD19). The genetically modifying step may be conducted in vivo or ex vivo. In some embodiments, the genetically modifying step is conducted ex vivo. The method may further include activation and/or expansion of the host cell ex vivo before, after and/or during the genetic modification.

### Isolation/Enrichment

The host cells may be autologous/autogeneic (“self”) or non-autologous (“non-self,” e.g., allogeneic, syngeneic or xenogeneic). In certain embodiments, the host cells are obtained from a mammalian subject. In other embodiments, the host cells are obtained from a primate subject. In certain embodiments, the host cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In certain embodiments, lymphocytes can be obtained from blood collected from a subject using techniques generally known to the skilled person, such as sedimentation, e.g., FICOLL™ separation.

In certain embodiments, cells from the circulating blood of a subject are obtained by apheresis. An apheresis device typically contains lymphocytes, including T-cells, monocytes, granulocytes, B cells, other nucleated white blood cells, red blood cells, and platelets. In certain embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In certain embodiments, host cells can be isolated from peripheral blood mononuclear cells (PBMCs) by lysing the red blood cells and depleting the monocytes. As an example, the cells can be sorted by centrifugation through a PERCOLL™ gradient. In certain embodiments, after isolation of PBMC, both cytotoxic and helper T lymphocytes can be sorted into naïve, memory, and effector T-cell subpopulations either before or after activation, expansion, and/or genetic modification.

In certain embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD3δ, CD45RA, CD45RO, CD56, CD62, CD62L, CD122, CD123, CD127, CD235a, CCR7, HLA-DR or a combination thereof using either positive or negative selection techniques. In certain embodiments, the T lymphocytes for use in the compositions of the disclosure do not express or do not substantially express one or more of the following markers: CD57, CD244, CD160, PD-1, CTLA4, TIM3, and LAG3.

In certain embodiments, NK cells can be enriched. For example, a specific subpopulation of T lymphocytes, expressing one or more markers such as, but not limited to, CD2, CD16, CD56, CD57, CD94, CD122 or a combination thereof using either positive or negative selection techniques.

### Stimulation/Activation

In order to reach sufficient therapeutic doses of host cell compositions, host cells are often subjected to one or more rounds of stimulation/activation. In certain embodiments, a method of producing host cells for administration to a subject comprises stimulating the host cells to become activated in the presence of one or more stimulatory signals or agents (e.g., compound, small molecule, e.g., small organic molecule, nucleic acid, polypeptide, or a fragment, isoform, variant, analog, or derivative thereof). In certain embodiments, a method of producing host cells for administration to a subject comprises stimulating the host cells to become activated and to proliferate in the presence of one or more stimulatory signals or agents.

Host cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the T-cell based host cells can be activated by binding to an agent that activates CD3ζ.

In other embodiments, a CD2-binding agent may be used to provide a primary stimulation signal to the T-cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100). Other antibodies which bind to the same epitopes as any of the above described antibodies can also be used.

In certain embodiments, the host cells are activated by administering phorbol myristate acetate (PMA) and ionomycine. In certain embodiments, the host cells are activated by administering an appropriate antigen that induces activation and then expansion. In certain embodiments, PMA, ionomycin, and/or appropriate antigen are administered with CD3 induce activation and/or expansion.

In general, the activating agents used in the present disclosure includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab)2′-fragment, diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). The divalent antibody fragment may be an (Fab)2′-fragment, or a divalent single-chain Fv fragment while the monovalent antibody fragment may be selected from the group consisting of a Fab fragment, a Fv fragment, and a single-chain Fv fragment (scFv).

In certain embodiments, one or more binding sites of the CD3ζ agents may be a bivalent proteinaceous artificial binding molecule such as a dimeric lipocalin mutein (i.e., duocalin). In certain embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In certain embodiments, the activating agent (e.g., CD3-binding agents) can be present in a concentration of about 0.1 to about 10 μg/ml. In certain embodiments, the activating agent (e.g., CD3-binding agents) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 g/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the activating agent (e.g., CD3-binding agents) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the CD3-binding agents can be present in a concentration of 1 μg/ml.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, 8,877,182; U.S. Patent Applications US2004/0058445, US2007/0160578, US2013/0011376, US2015/0118207, US2015/0037887; and PCT Patent Application WO2016/122147, each of which is incorporated herein by reference in its entirety.

In certain embodiments, the NK based host cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LTLRB5 receptor).

In certain embodiments, the NK based host cells can be activated by, for example and not limitation, feeder cells (e.g., native K562 cells or K562 cells that are genetically modified to express 4-1BBL and cytokines such as IL15 or IL21).

In other embodiments, interferons or macrophage-derived cytokines can be used to activate NK cells. For example and not limitation, such interferons include but are not limited to interferon alpha and interferon gamma, and such cytokines include but are not limited to TL-15, IL-2, IL-21.

In certain embodiments, the NK activating agent can be present in a concentration of about 0.1 to about 10 μg/ml. In certain embodiments, the NK activating agent can be present in a concentration of about 0.2 μg/ml to about 9 μg/ml, about 0.3 μg/ml to about 8 μg/ml, about 0.4 g/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the NK activating agent is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the NK activating agent can be present in a concentration of 1 μg/ml.

In certain embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

### Polynucleotide Transfer

In certain embodiments, the host cells are genetically modified to express a CAR described above. The host cells can be genetically modified after stimulation/activation. In certain embodiments, the host cells are modified within 12 hours, 16 hours, 24 hours, 36 hours, or 48 hours of stimulation/activation. In certain embodiments, the cells are modified within 16 to 24 hours after stimulation/activation. In certain embodiments, the host cells are modified within 24 hours.

In order to genetically modify the host cell to express the CAR, the CAR polynucleotide construct must be transferred into the host cell. Polynucleotide transfer may be via viral or non-viral gene methods. Suitable methods for polynucleotide delivery for use with the current methods include any method known by those of skill in the art, by which a polynucleotide can be introduced into an organelle, cell, tissue or organism.

In some embodiments, polynucleotides are transferred to the cell in a non-viral vector. In some embodiments, the non-viral vector is a transposon. Exemplary transposons hat can be used in the present disclosure include, but are not limited to, a sleeping beauty transposon and a PiggyBac transposon.

Nucleic acid vaccines can be used to transfer CAR polynucleotides into the host cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In certain embodiments, the host cells can be genetically modified by methods ordinarily used by one of skill in the art. In certain embodiments, the host cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety.

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244(4910):1342-1344, 1989, both of which are incorporated herein by reference in their entity. In certain embodiments, the host cells may be removed from the subject and transfected ex vivo using the polynucleotides (e.g., expression vectors) of the disclosure. In certain embodiments, the host cells obtained from the subject can be transfected or transduced with the polynucleotides (e.g., expression vectors) of the disclosure and then administered back to the subject.

Another method of gene transfer includes injection. In certain embodiments, a cell or a polynucleotide or viral vector may be delivered to a cell, tissue, or organism via one or more injections (e.g., a needle injection). Non-limiting methods of injection include injection of a composition (e.g., a saline based composition). Polynucleotides can also be introduced by direct microinjection. Non-limiting sites of injection include, subcutaneous, intradermal, intramuscular, intranodal (allows for direct delivery of antigen to lymphoid tissues). intravenous, intraprostatic, intratumor, intralymphatic (allows direct administration of DCs) and intraperitoneal. It is understood that proper site of injection preparation is necessary (e.g., shaving of the site of injection to observe proper needle placement).

Electroporation is another method of polynucleotide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In certain embodiments, cell wall-degrading enzymes, such as pectin-degrading enzymes, can be employed to render the host cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this disclosure include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Additional methods of polynucleotide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In further embodiments, host cells are genetically modified using gene editing with homology-directed repair (HDR). Homology-directed repair (HDR) is a mechanism used by cells to repair double strand DNA breaks. In HDR, a donor polynucleotide with homology to the site of the double strand DNA break is used as a template to repair the cleaved DNA sequence, resulting in the transfer of genetic information from the donor polynucleotide to the DNA. As such, new nucleic acid material may be inserted or copied into a target DNA cleavage site. Double strand DNA breaks in host cells may be induced by a site-specific nuclease. The term “site-specific nuclease” as used herein refers to a nuclease capable of specifically recognizing and cleaving a nucleic acid (DNA or RNA) sequence. Suitable site-specific nucleases for use in the present disclosure include, but are not limited to, RNA-guided endonuclease (e.g., CRISPR-associated (Cas) proteins), zinc finger nuclease, a TALEN nuclease, or mega-TALEN nuclease. For example, a site-specific nuclease (e.g., a Cas9+ guide RNA) capable of inducing a double strand break in a target DNA sequence is introduced to a host cell, along with a donor polynucleotide encoding a CAR of the present disclosure and optionally an additional protein (e.g., tCD19).

### Expansion/Proliferation

After the host cells are activated and transduced, the cells are cultured to proliferate. T-cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion. Agents that can be used for the expansion of T-cells can include interleukins, such as IL-2, IL-7, IL-15, or IL-21 (see for example Cornish et al. 2006, Blood. 108(2):600-8, Bazdar and Sieg, 2007, Journal of Virology, 2007, 81(22):12670-12674, Battalia et al, 2013, Immunology, 139(1):109-120). Other illustrative examples for agents that may be used for the expansion of T-cells are agents that bind to CD8, CD45 or CD90, such as αCD8, αCD45 or αCD90 antibodies. Illustrative examples of T-cell population including antigen-specific T-cells, T helper cells, cytotoxic T-cells, memory T-cell (an illustrative example of memory T-cells are CD62L|CD8| specific central memory T-cells) or regulatory T-cells (an illustrative example of Treg are CD4+CD25+CD45RA+ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the agent(s) used for expansion (e.g., IL-2) are administered at about 20 units/ml to about 200 units/ml. In certain embodiments, the agent(s) used for expansion (e.g., IL-2) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In certain embodiments, the agent(s) used for expansion (e.g., IL-2) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In certain embodiments, the agent(s) used for expansion (e.g., IL-2) are administered at about 5 mg/ml to about 10 ng/ml. In certain embodiments, the agent(s) used for expansion (e.g., IL-2) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In certain embodiments, the agent(s) used for expansion (e.g., IL-2) are administered at about 5 ng/ml, 6 ng/ml, 7 ng/ml, 8 ng/ml, 9, ng/ml, or 10 ng/ml.

After the host cells are activated and transduced, the cells are cultured to proliferate. NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of natural killer cells can include agents that bind to CD16 or CD56, such as for example αCD16 or αCD56 antibodies. In certain embodiments, the binding agent includes antibodies (see for example Hoshino et al, Blood. 1991 Dec. 15; 78(12):3232-40.). Other agents that may be used for expansion of NK cells may be IL-15 (see for example Vitale et al. 2002. The Anatomical Record. 266:87-92, which is hereby incorporated by reference in its entirety for all purposes).

Conditions appropriate for T-cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for host cell expansion include, but are not limited to, surfactant, piasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In certain embodiments, host cells of the present disclosure may be modified such that the expression of an endogenous TCR, MHC molecule, or other immunogenic molecule is decreased or eliminated. When allogeneic cells are used, rejection of the therapeutic cells may be a concern as it may cause serious complications such as the graft-versus-host disease (GvHD). Although not wishing to be bound by theory, immunogenic molecules (e.g., endogenous TCRs and/or MHC molecules) are typically expressed on the cell surface and are involved in self vs non-self discrimination. Decreasing or eliminating the expression of such molecules may reduce or eliminate the ability of the therapeutic cells to cause GvHD.

In certain embodiments, expression of an endogenous TCR in the host cells is decreased or eliminated. In a particular embodiment, expression of an endogenous TCR (e.g., ap TCR) in the host cells is decreased or eliminated. Expression of the endogenous TCR may be decreased or eliminated by disrupting the TRAC locus, TCR beta constant locus, and/or CD3 locus. In certain embodiments, expression of an endogenous TCR may be decreased or eliminated by disrupting one or more of the TRAC, TRBC1, TRBC2, CD3E, CD3G, and/or CD3D locus.

In certain embodiments, expression of one or more endogenous MHC molecules in the host cells is decreased or eliminated. Modified MHC molecule may be an MHC class I or class II molecule. In certain embodiments, expression of an endogenous MHC molecule may be decreased or eliminated by disrupting one or more of the MHC, β2M, TAP1, TAP2, CIITA, RFX5, RFXAP and/or RFXANK locus.

Expression of the endogenous TCR, an MHC molecule, and/or any other immunogenic molecule in the host cell can be disrupted using genome editing techniques such as Clustered regularly interspaced short palindromic repeats (CRISPR)/Cas, zinc finger nucleases (ZFNs), transcription activator-like effector nucleases (TALENs), and Meganucleases. These genome editing methods may disrupt a target gene by entirely knocking out all of its output or partially knocking down its expression. In a particular embodiment, expression of the endogenous TCR, an MHC molecule and/or any other immunogenic molecule in the host cell is disrupted using the CRISPR/Cas technique.

### Pharmaceutical Compositions

In some embodiments, the compositions comprise one or more polypeptides of the CARs and other related molecules (e.g., second CAR or bispecific molecule), polynucleotides, vectors comprising same, and cell compositions, as disclosed herein. Compositions of the present disclosure include, but are not limited to pharmaceutical compositions.

In one aspect, the present disclosure provides a pharmaceutical composition comprising a polynucleotide or a recombinant vector described herein, and a pharmaceutically accepted carrier and/or excipient.

In another aspect, the present disclosure provides pharmaceutical composition comprising the CAR-modified host cells described herein and a pharmaceutically acceptable carrier and/or excipient. In some embodiments, the host cells are modified with a Col11A1-binding CAR. In some embodiments, the host cells are modified with a C.TNC-binding CAR. In some embodiments, the host cells are modified with a Col11A1-binding CAR and a C.TNC-binding CAR.

In another aspect, the present disclosure provides pharmaceutical composition comprising host cells modified with a Col11A1-binding CAR and host cells modified with a C.TNC-binding CAR, and a pharmaceutically acceptable carrier and/or excipient.

Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising CAR-modified host cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising CAR-modified host cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated for parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the CAR-modified host cells may be mixed with substances that adhere or penetrate then prior to their administration, e.g., but not limited to, nanoparticles.

### Therapeutic Methods

In one aspect, the present disclosure provides a method for treating a tumor in a subject in need thereof. A therapeutically effective amount of the CAR-modified host cells described herein or the pharmaceutical composition comprising the host cells is administered to the subject.

The term “tumor” refers to a benign or malignant abnormal growth of tissue. The term “tumor” includes cancer. Examples of tumors are, but not limited to, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of tumors can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internetwebsite of Merck Manuals); and SEER Program Coding and Staging Manual 2016, each of which are incorporated by reference in their entirety for all purposes.

In some embodiments, host cells modified with a Col11A1-binding CAR, or pharmaceutical compositions thereof, are administered to a subject to treat a tumor expressing a Col11A1 splice variant. Non-limiting examples of tumors expressing a Col11A1 splice variant include acute lymphoblastic leukemia, acute myeloid leukemia, adult solid tumors and brain tumors, adrenal gland tumors, anal cancer, bile duct cancer, bladder cancer, blood cancers, bone cancer, bowel cancer, brain tumors, breast cancer, cancer of unknown primary, cancer spread to bone, cancer spread to brain, cancer spread to liver, cancer spread to lung, carcinoid, cervical cancer, children's cancers, colorectal cancer, chronic lymphocytic leukemia, chronic myeloid leukemia, colorectal cancer, ear cancer, endometrial cancer, eye cancer, follicular dendritic cell sarcoma, gallbladder cancer, gastric cancer, gastro esophageal junction cancers, germ cell tumors, gestational trophoblastic disease, glioma, glioblastoma, gynecological cancer, hairy cell leukemia, head and neck squamous cell carcinoma, high grade gliomas, Hodgkin lymphoma, Kaposi's sarcoma, kidney cancer, large bowel and rectal neuroendocrine tumors, laryngeal cancer, leukemia, Linitis plastica of the stomach, liver cancer, low grade gliomas, lung cancer, lung neuroendocrine tumors (NETs), lymphoma, malignant schwannoma, mediastinal germ cell tumors, melanoma, men's cancer, merkel cell skin cancer, mesothelioma, molar pregnancy, mouth and oropharyngeal cancer, myeloma, nasal and paranasal sinus cancer, nasopharyngeal cancer, neuroblastoma, neuroendocrine tumors, neuroendocrine tumors of the pancreas, non-Hodgkin lymphoma, non-Hodgkin lymphoma in children, esophageal cancer, oral squamous cell carcinoma, ovarian cancer, pancreatic cancer, pediatric solid tumors and brain tumors, penile cancer, persistent trophoblastic disease and choriocarcinoma, pheochromocytoma, prostate cancer, pseudomyxoma peritonei, rare cancers, rectal cancer, renal cancer, retinoblastoma, salivary gland cancer, secondary cancer, signet cell cancer, skin cancer, small bowel cancer, small bowel neuroendocrine tumors, soft tissue sarcoma, stomach cancer, stomach neuroendocrine tumors, testis cancer, thymus gland tumors, thyroid cancer, tongue cancer, tonsil cancer, tumors of the adrenal gland, unknown primary cancer, urothelial, uterine cancer, vaginal cancer, vulval cancer, Wilms' tumor, and womb cancer.

In some embodiments, host cells modified with a C.TNC-binding CAR, or pharmaceutical compositions thereof, are administered to a subject to treat a tumor expressing C.TNC splice variant. Non-limiting examples of tumors expressing a C.TNC splice variant include breast cancer, brain tumors such as, but not limited to, glioblastoma, high grade gliomas, low grade gliomas, head and neck cancers, liver cancers, lung cancers, melanoma, ovarian cancer, pancreatic cancer, prostate cancer, renal cancer, urothelial cancer, carcinoid, cervical cancers, colorectal cancer, endometrial cancer, lymphoma, skin cancer, stomach cancer, testis cancer, thyroid cancer and urothelial cancer.

In some embodiments, host cells modified a Col11A1-binding CAR and a C.TNC-binding CAR, or pharmaceutical compositions thereof, may be administered to a subject to treat any tumor described above.

In cases where the CAR-modified host cells also express a CD20 polypeptide, the method may further include administering an anti-CD20 antibody to the subject for removal of the isolated host cells. The anti-CD20 antibody is administered in an amount effective for sufficient removal of the isolated host cells from the subject. In some embodiments, the anti-CD20 antibody is administered in an amount effective for removal of more than 50% of the isolated host cells from the subject. For example, the anti-CD20 antibody may be administered in an amount effective for removal of more than 55%, more than 60%, more than 65%, more than 70%, more than 75%, more than 80%, more than 85%, more than 90%, more than 95%, more than 98%, more than 99%, or about 100% of the isolated host cells from the subject. The anti-CD20 antibody may be administered in an amount effective for removal of about 50% to about 70%, about 60% to about 80%, about 70% to about 90%, or about 80% to about 100% of the isolated host cells from the subject.

Non-limiting examples of anti-CD20 antibodies that can be used for removal the isolated host cells include Rituximab, Ibritumomab tiuxetan, Tositumomab, Ofatumumab, Ocrelizumab, TRU-015, Veltuzumab, AME-133v, PRO131921, and Obinutuzumab. In some embodiments, the anti-CD20 antibody is Rituximab.

In some embodiments, the therapeutic method of the present disclosure includes one or more of the following steps: (a) isolating immune cells from the subject or donor; (b) modifying the immune cells ex vivo with a polynucleotide encoding a CAR and optionally an additional protein, a second CAR and/or a bispecific molecule, or a recombinant vector comprising the same; (c) optionally, expanding and/or activating the modified immune cells before, after and/or during step (b); (d) introducing a therapeutically effective amount of the modified immune cells into the subject, and (e) in cases when the modified immune cells comprise the CD20 suicide switch, optionally, administering an anti-CD20 antibody to the subject, wherein the anti-CD20 antibody is administered in an amounts effective for removal of the modified immune cells from the subject. The immune cells may be T-cells and/or NK cells.

In some embodiments, the modified host cell is an autologous cell. In some embodiments, the modified host cell is an allogeneic cell. In cases where the host cell is isolated from a donor, the method may further include a method to prevent graft vs host disease (GVHD) and the host cell rejection.

In some embodiments of any of the therapeutic methods described above, the composition is administered in a therapeutically effective amount. The dosages of the composition administered in the methods of the disclosure will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of modified host cells. It is also contemplated that a variety of doses will be effective to improve in vivo effector function of modified host cells.

In some embodiments, composition comprising the modified host cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of modified host cells will depend on the therapeutic use for which the composition is intended for.

Modified host cells may be administered multiple times at dosages listed above. The modified host cells may be allogeneic, syngeneic, xenogeneic, or autologous to the patient undergoing therapy.

The compositions and methods described in the present disclosure may be utilized in conjunction with other types of therapy for tumors, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., IFN or IL-2).

As a non-limiting example, the disclosure can be combined with other therapies that block inflammation (e.g., via blockage of IL1, INFα/β, IL6, TNF, IL23, etc.).

The methods and compositions of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the disclosure can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the disclosure can also be combined with other treatments such as midostaurin, enasidenib, or a combination thereof.

Therapeutic methods of the disclosure can be combined with additional immunotherapies and therapies. For example, when used for treating tumors, the compositions of the disclosure can be used in combination with conventional therapies, such as, e.g., surgery, radiotherapy, chemotherapy or combinations thereof, depending on type of the tumor, patient condition, other health issues, and a variety of factors. In certain aspects, other therapeutic agents useful for combination tumor therapy with the inhibitors of the disclosure include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the modified host cells of the disclosure can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-tumor agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a human. The subject may be a juvenile or an adult, of any age or sex.

In accordance with the present disclosure there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ.

### EXAMPLES

The following examples are provided to further describe some of the embodiments disclosed herein. The examples are intended to illustrate, not to limit, the disclosed embodiments.

### Example 1: Col11A1 Splice Variant Expression in Pediatric Cancer

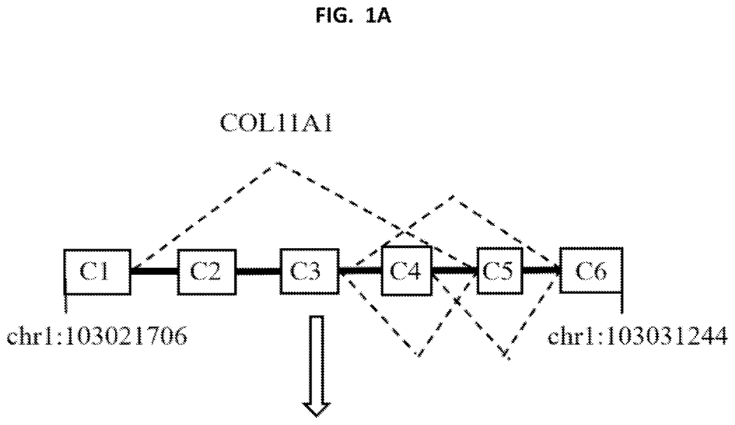

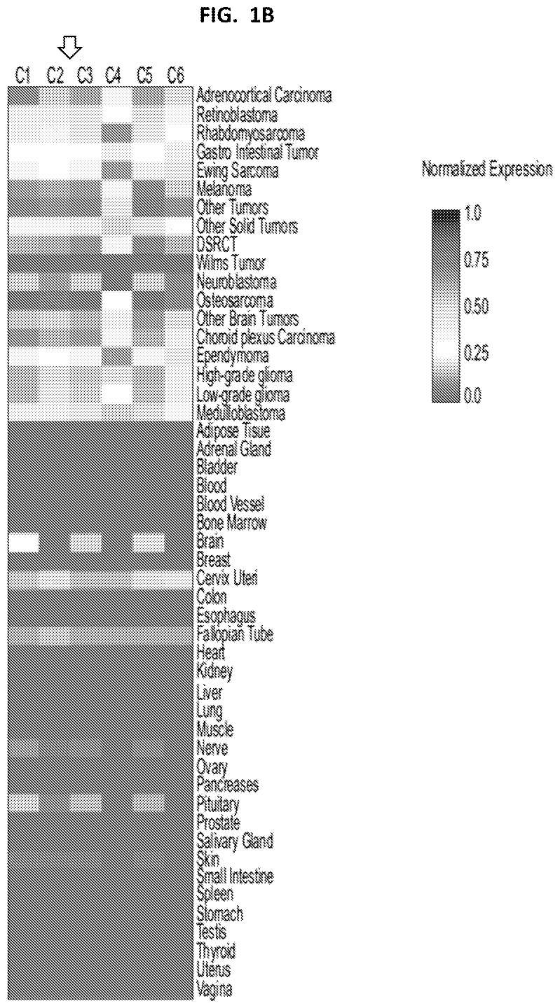

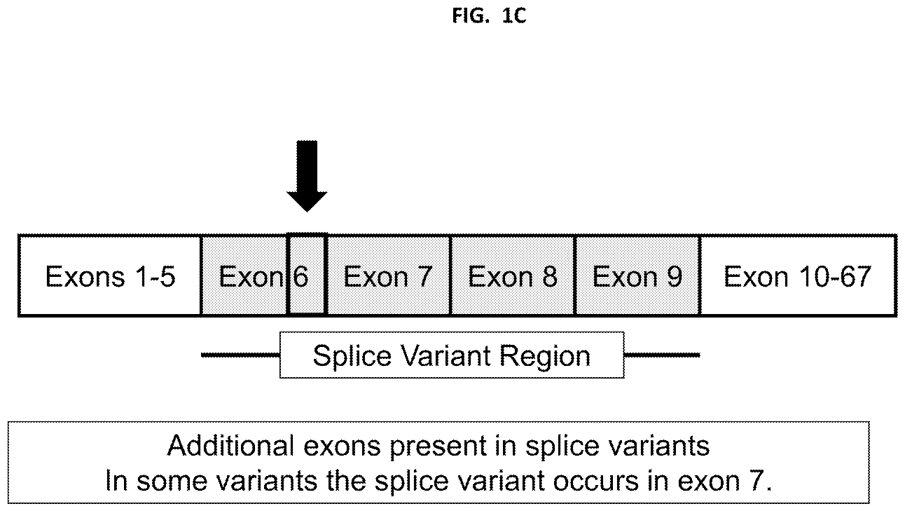

To evaluate Col11A1 splice variant expression in pediatric cancer, RNAseq reads were processed by two-pass STAR mapping followed by HTseq exon quantification. Gene abundance was measured in the number of fragments per kilobase of transcripts per million mapped reads (FPKM), and ranked normalized on a heatmap to allow for visualization Col11A1 exon expression, as displayed in FIG. 1B. Each cell of the heatmap shows the sample median for each pediatric tumor and normal (non-cancerous) tissue. RNAseq from pediatric solid and brain tumors were used to quantify tumor exon expression. GTEx RNAseq samples were used to quantify exon expression in normal (non-cancerous) tissue.

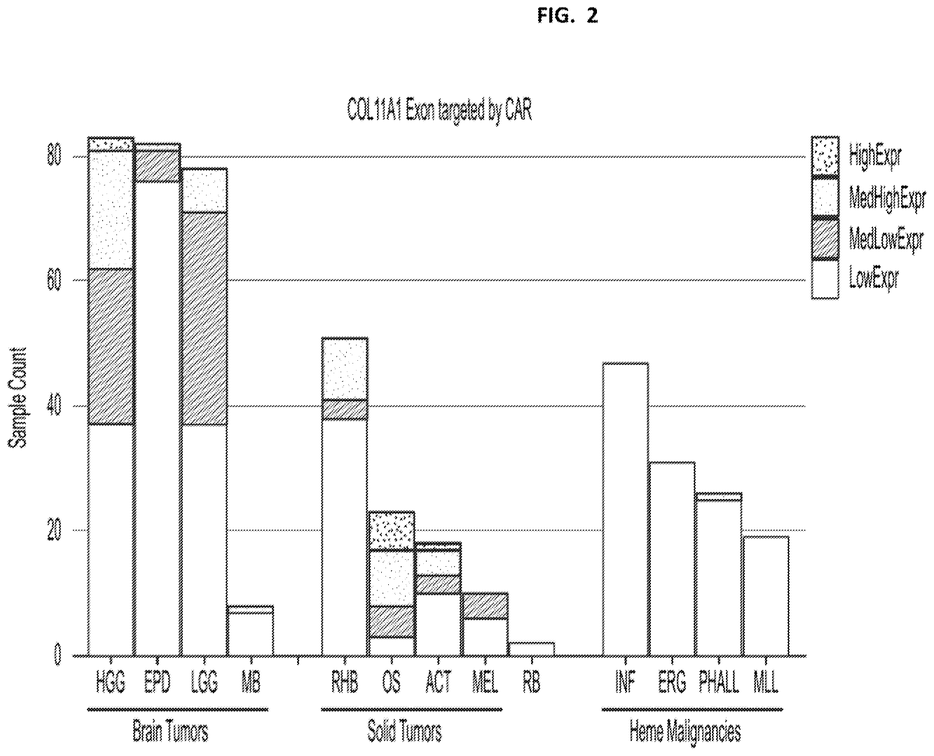

Col11A1 expression was also quantified by quartiles using data from the Pediatric Cancer Genome Project, as shown in FIG. 2. Briefly, pediatric tumor samples were characterized based on RNA expression of the Col11A1 exon that is targeted by the CAR as either high expression (Q4: greater than 75%), medium-high expression (Q3: 50-70%), medium-low expression (Q2: 25-50%), or low expression (Q1: less than 25%). Brain tumors evaluated in this analysis were high grade glioma (HGG), ependymoma (EPD), low grade glioma (LGG), and medulloblastoma (MB). Solid tumors evaluated in this analysis were rhabdomyosarcoma (RHB), osteosarcoma (OS), adrenocortical carcinoma (ACT), melanoma (MEL), and retinoblastoma (RB). Heme malignancies evaluated in this analysis were infant all (INF), B-ALL with ERG alterations (ERG), Philadelphia like acute lymphoblastic leukemia (PHALL), and mixed lineage leukemia (MLL). High expression (HighExpr) and/or medium-high expression (MedHighExpr) of the Col11A1 exon was prevalent in HGG and LGG included in the analysis, but was also observed for several of the solid tumors (e.g., RHB, OS, MEL, and ACT). Medium-low expression (MedLowExpr) of the Col11A1 exon was also observed for each of the brain tumors. For solid tumors, medium-low expression of the Col11A1 exon was observed in RHB, OS, ACT and Mel. Each of the heme malignancies showed only low expression (LowExpr) of the Col11A1 exon, as was also documented for each of the brain tumors and solid tumors.

### Example 2: Generation of Col11A1-CAR T-Cells

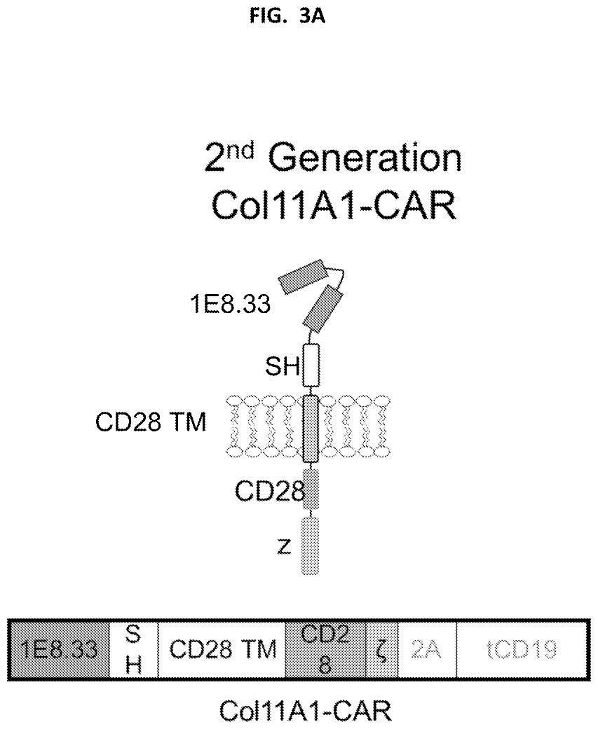

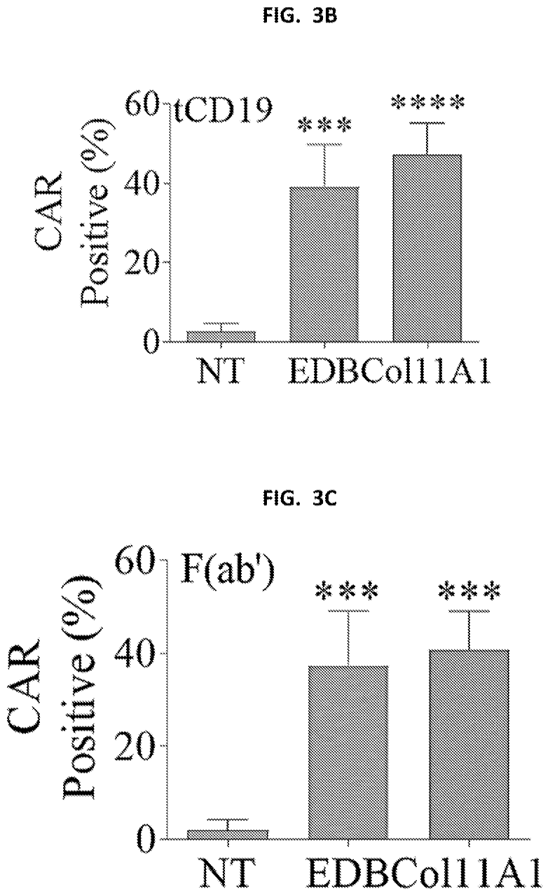

A retroviral vector was designed encoding an Col11A1-specific CAR (Col11A1-CAR) using a Col11A1-specific scFv (1E8.33) that has shown tumor specificity human imaging studies (see, e.g., U.S. Pat. No. 9,702,879, the content of which is herein incorporated by reference in its entirety), a CD28hinge/transmembrane domain (CD28H/TM) and a CD28.ζ signaling domain, as schematically represented in FIG. 3A. Col11A1-CAR T-cells were generated by retroviral transduction of CD3/CD28-activated T-cells in the presence of IL-7 (10 ng/ml) and IL-15 (10 ng/ml). CAR expression was detected on transduced T-cells by fluorescence-activated cell sorting (FACS) analysis for truncated CD19 (tCD19), and for anti-F(ab)′, as shown in FIG. 3B and FIG. 3C, respectively (n=4 donors, ***p<0.001, ****p<0.0001, 2-way ANOVA).

### Example 3: Col11A1-CAR Recognition and Killing of Col11A1+ Tumor Cells In Vitro

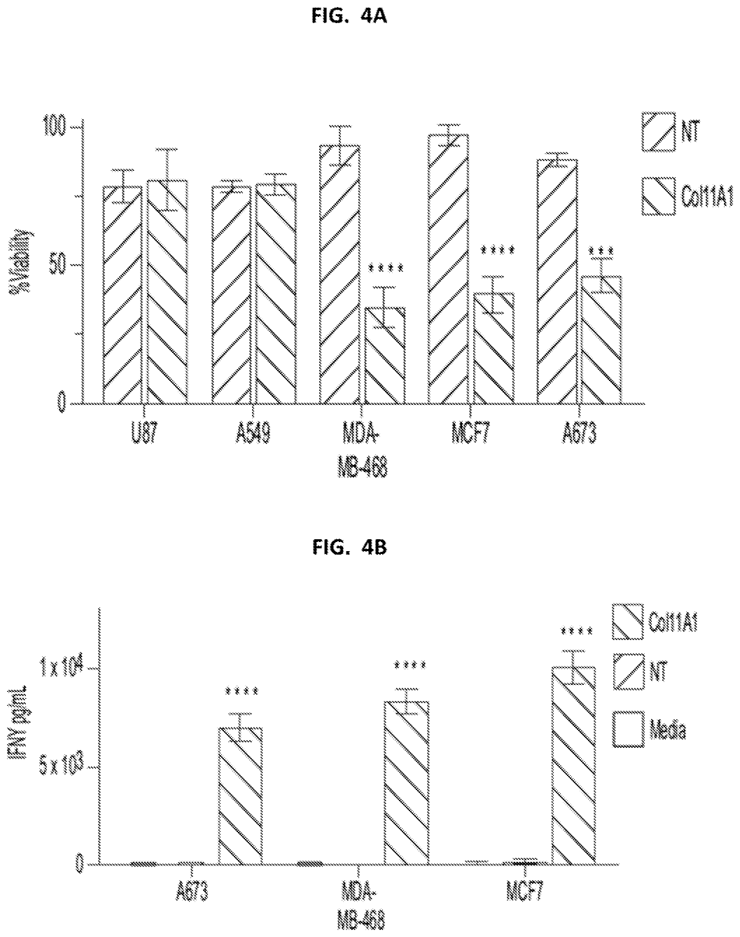

To evaluate Col11A1-CAR T-cells recognition and killing of Col11A1+ tumor cells in vitro, multiple cell lines such as, but not limited to, U87 (high grade glioma) cells, A549 (lung cancer) cells, MDA-MB-468 and MCF7 (breast cancer) cells, and A673 (Ewing's sarcoma) cells were tested. Cytolytic activity of CAR and non-transduced (NT) T-cells was determined by standard MTS assay at 4:1 E:T (effector to target cell) ratio for 3 days. Data demonstrating COL11A1-CAR T cell-induced cell death in breast cancer and Ewing's sarcoma cell lines are displayed in FIG. 4A. To measure IFNγ secretion, 5×10⁵ tumor cells (e.g., A673, MDA-MB-468, and MCF7) and 1×10⁶ T-cells were co-cultured in wells of a 24-well tissue culture plate. After 24 hours, the cell culture media was harvested and IFNγ production (pg/mL) was determined by enzyme-linked immunosorbent assay (ELISA), as shown in FIG. 4B (n=3 donors, ***p<0.001, ****p<0.0001, 2-way ANOVA). The results showed that IFNγ secretion was higher in COL11A1-CAR-T-cell co-cultures than in NT co-culture and media control conditions, across tumor cell types.

### Example 4: Col11A1 Recognition and Killing of Col11A1+ Tumor Cells In Vivo

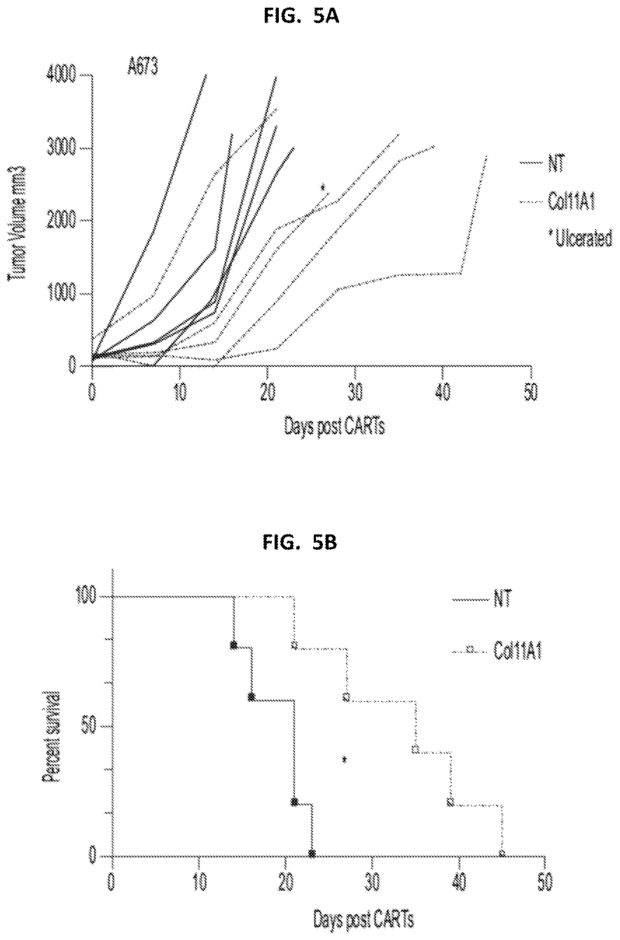

To investigate Col11A1 recognition and killing of Col11A1+ tumor cells in vivo, A673 Ewing's sarcoma cells (2×10⁶ cells) were injected subcutaneously (s.c.) into immunodeficient NOD scid gamma (NSG) mice, and on day 10, mice received a single intravenous injection of 1×10⁶ of either CoL11A1-CAR T cells or NT T-cells. Tumor growth was measured (mm³) by serial caliper, as shown in FIG. 5A. Kaplan Meier percent survival data with statistically significant advantage are shown in FIG. 5B (n=5 mice, *p<0.05 Log-rank [Mantel-Cox test]). These data show that intravenous injection of CoL11A1-CAR T cell was associated with decreased tumor volume, and increased percent survival in NSG immunodeficient mice.

### Example 5: TNC C Domain (C.TNC) Expression in Pediatric Cancer

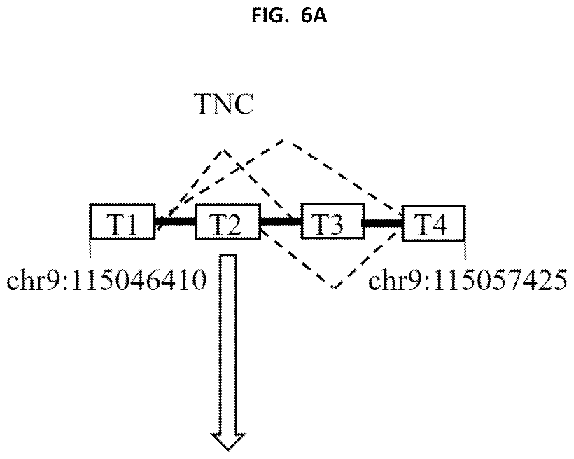

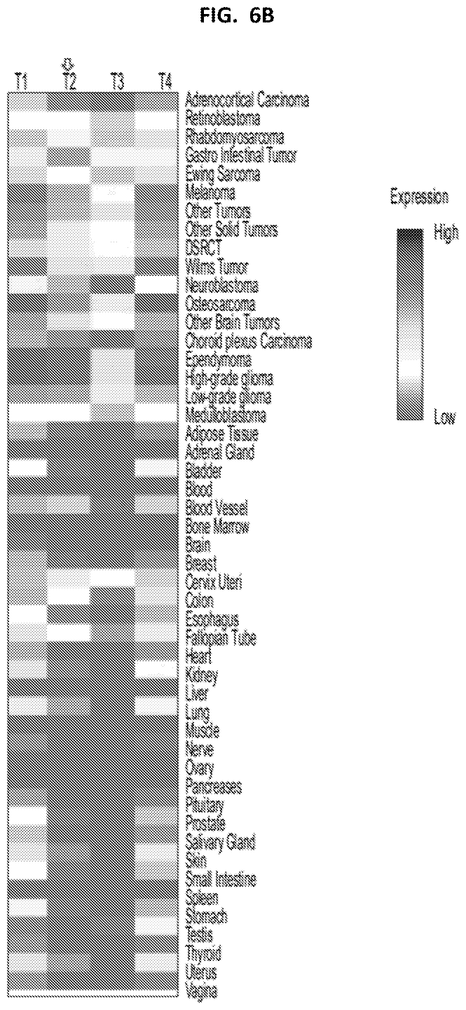

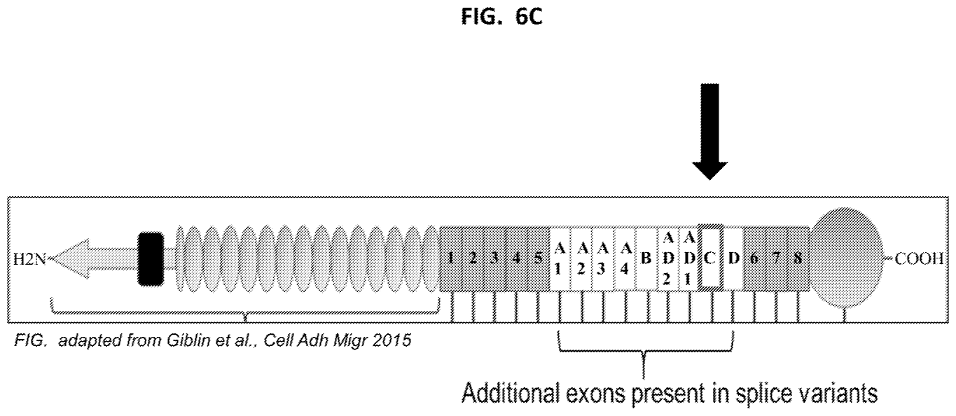

To evaluate C.TNC splice variant expression in pediatric cancer, as schematized in FIG. 6A, RNAseq reads were processed by two-pass STAR mapping followed by HTseq exon quantification. Gene abundance was measured in the number of fragments per kilobase of transcripts per million mapped reads (FPKM), and ranked normalized on a heatmap to allow for visualization of C.TNC exon expression, as displayed in FIG. 6B. Each cell of the heatmap shows the sample median for each pediatric tumor and normal (non-cancerous) tissue. RNAseq from pediatric solid and brain tumors were used to quantify tumors exon expression. GTEx RNAseq samples were used to quantify exon expression in normal (non-cancerous) tissue.

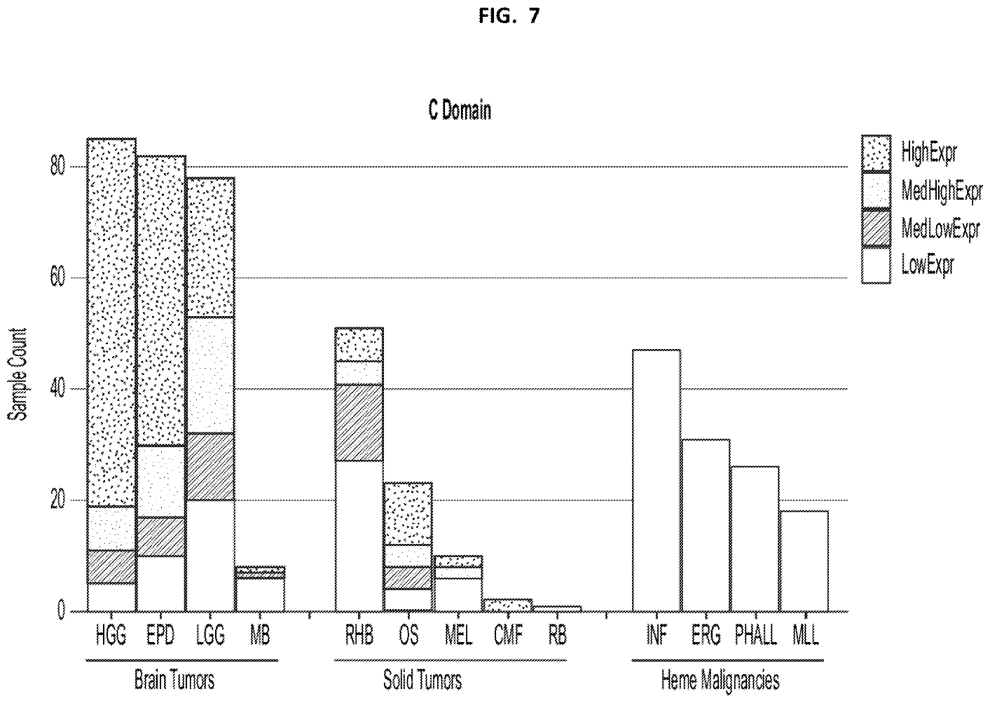

C.TNC expression was also quantified by quartiles using data from the Pediatric Cancer Genome Project, as shown in FIG. 7. Briefly, pediatric tumor samples were characterized based on RNA expression of the C domain of TNC as either high expression (Q4: greater than 75%), medium-high expression (Q3: 50-70%), medium-low expression (Q2: 25-50%), or low expression (Q1: less than 25%). Brain tumors evaluated in this analysis were high grade glioma (HGG), ependymoma (EPD), low grade glioma (LGG), and medulloblastoma (MB). Solid tumors evaluated in this analysis were rhabdomyosarcoma (RHB), osteosarcoma (OS), melanoma (MEL), chondromyxofibroma (CMF), and retinoblastoma (RB). Heme malignancies evaluated in this analysis were infant ALL (INF), B-ALL with ERG alterations (ERG), Philadelphia like acute lymphoblastic leukemia (PHALL), and mixed lineage leukemia (MLL). High expression (HighExpr) and/or medium-high expression (MedHighExpr) of C.TNC was prevalent for each of the brain tumors included in the analysis, but was also observed for several of the solid tumors (e.g., RHB, OS, MEL, and CMF). Medium-low expression (MedLowExpr) of C.TNC was also observed for each of the brain tumors. For solid tumors, medium-low expression of C.TNC was observed in RHB and OS cancers. Each of the heme malignancies showed only low expression (LowExpr) of C.TNC, as was also documented for each of the brain tumors and all but one (CMF) of the solid tumors.

### Example 6: C.TNC as a Target for CAR T Cells

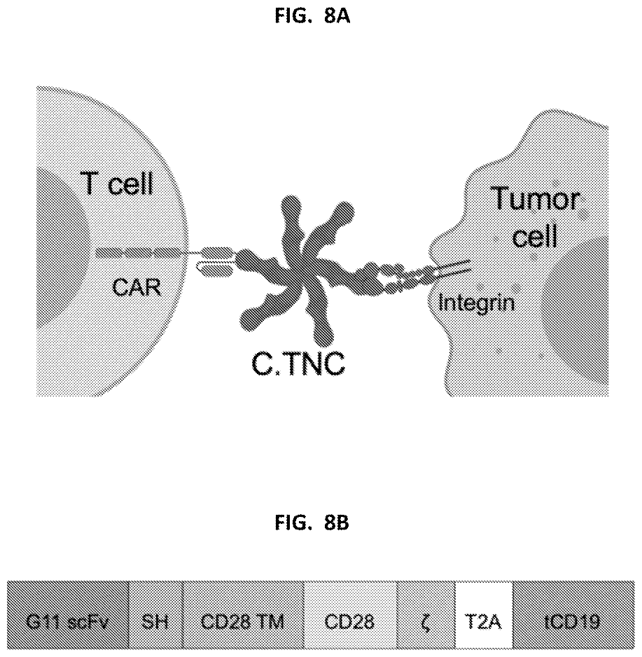

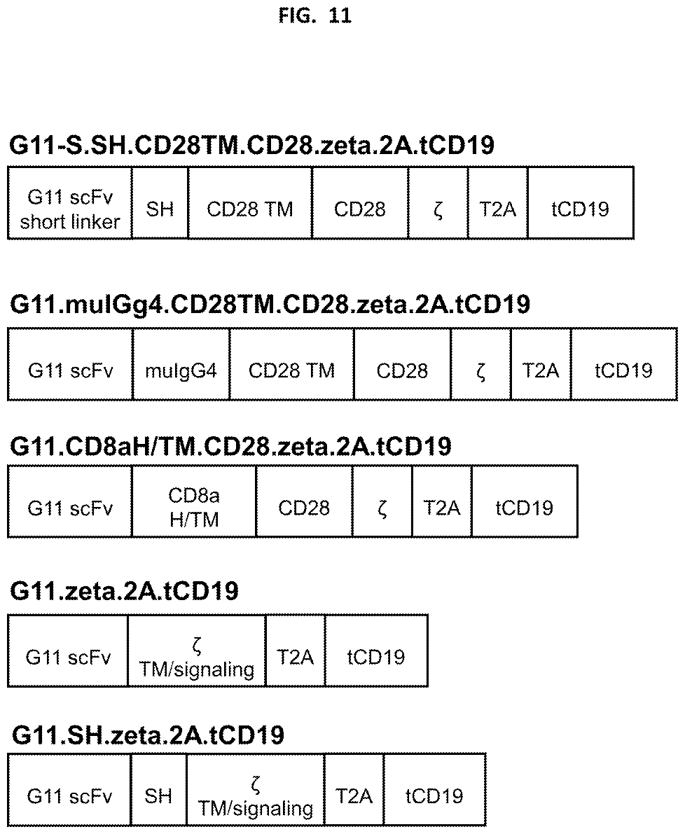

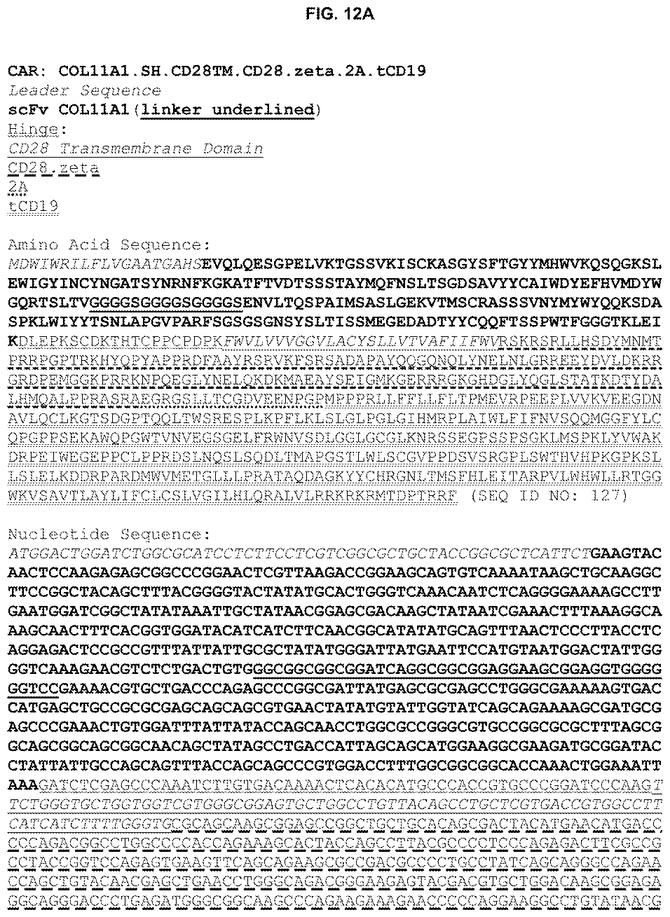

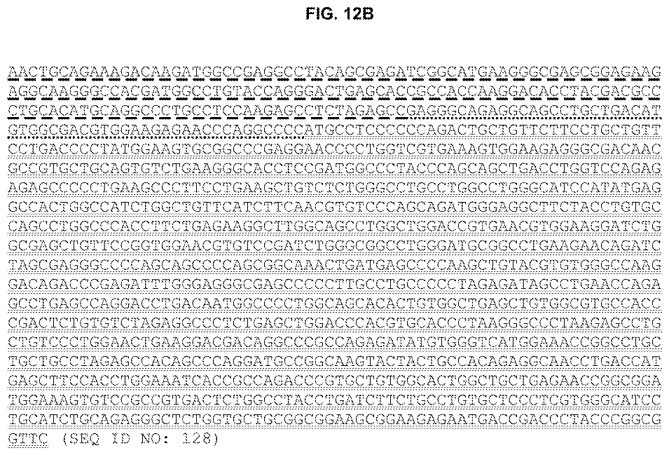

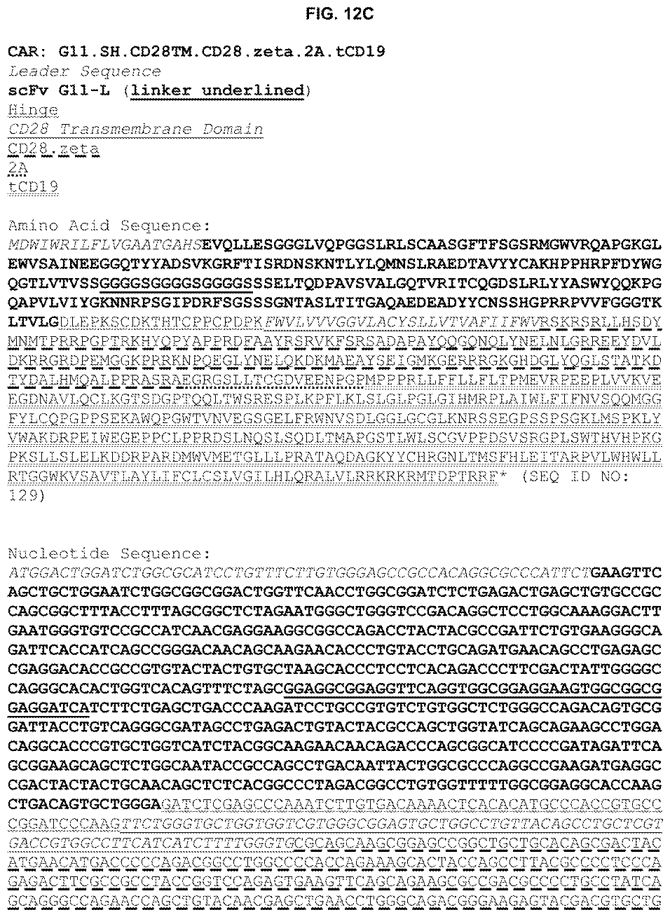

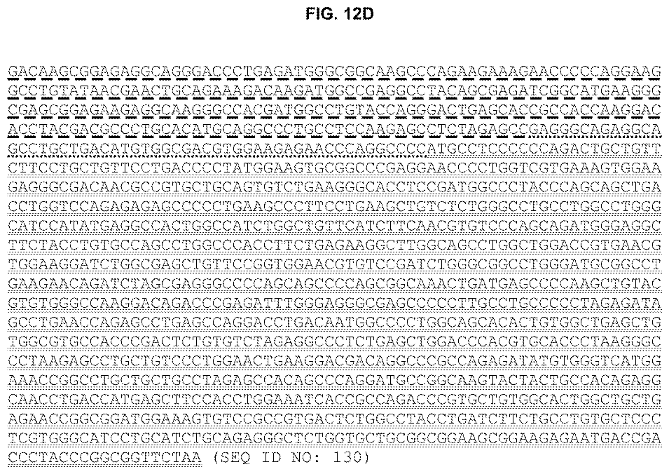

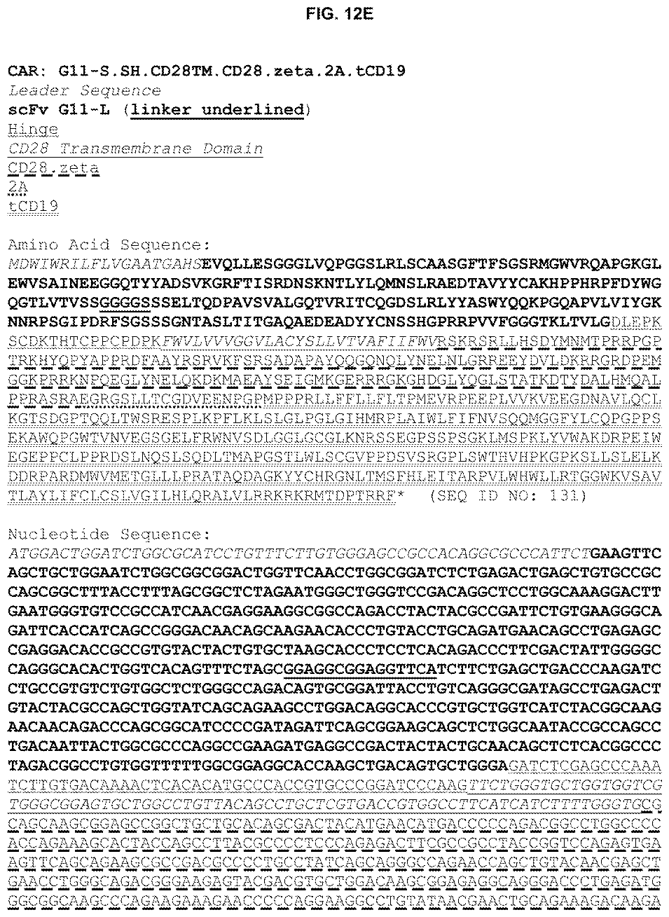

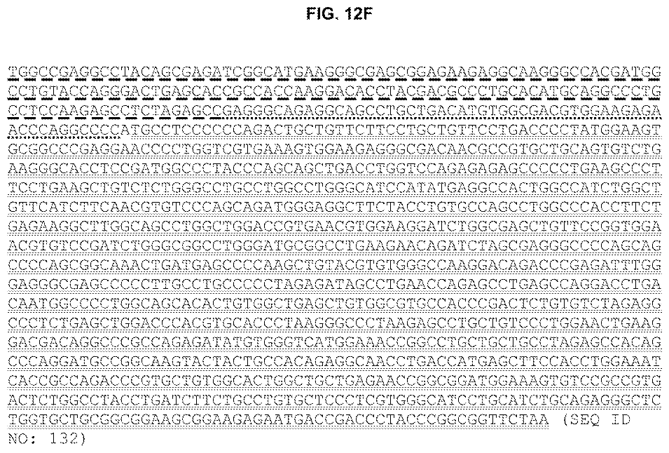

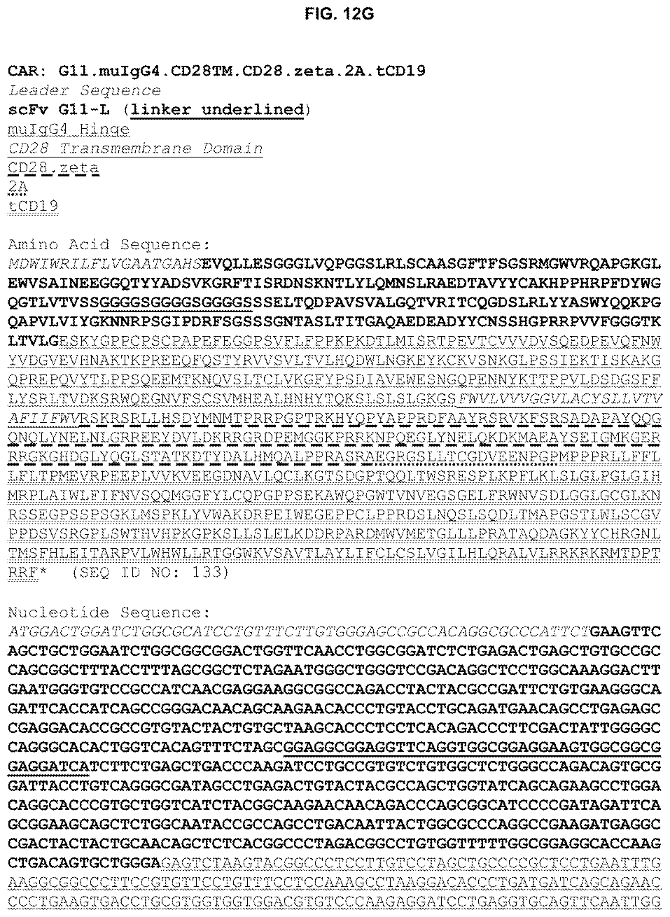

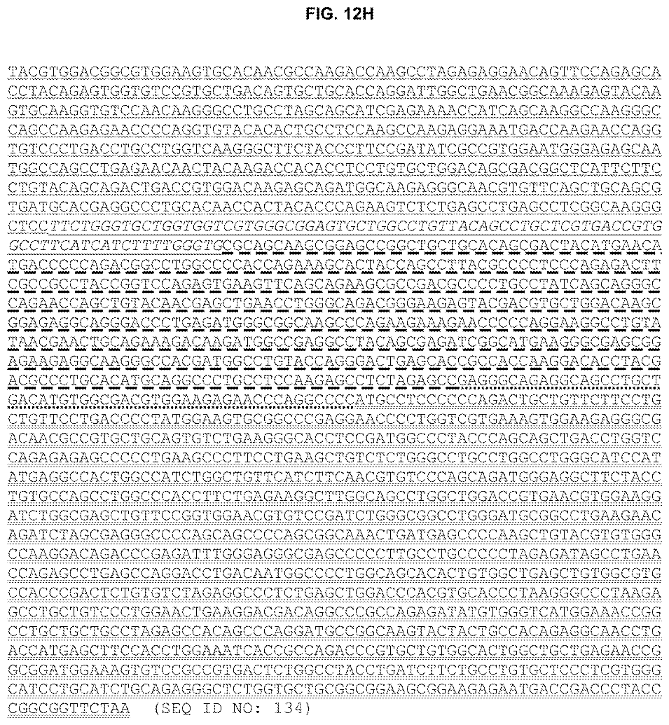

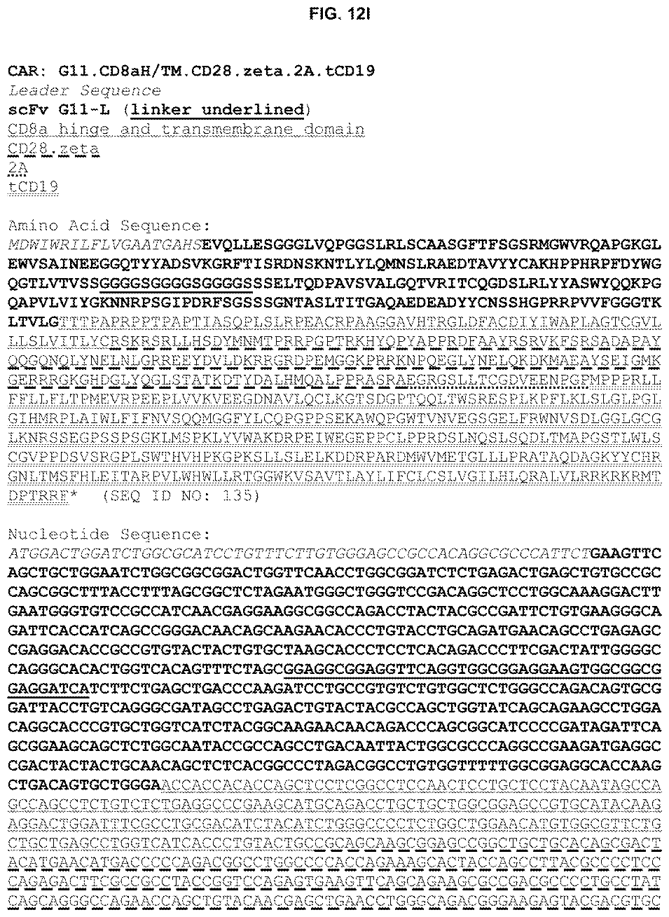

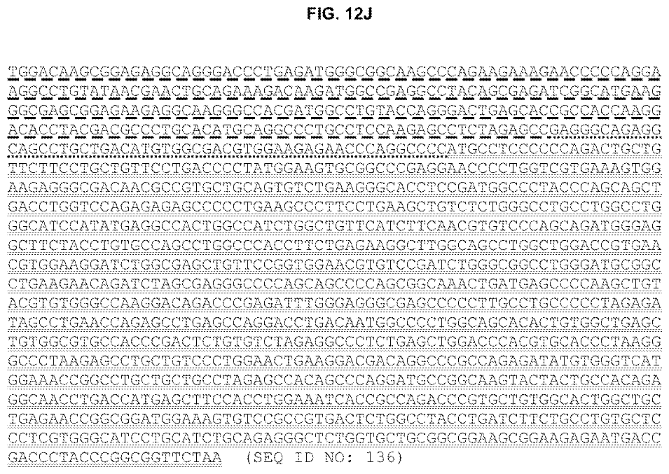

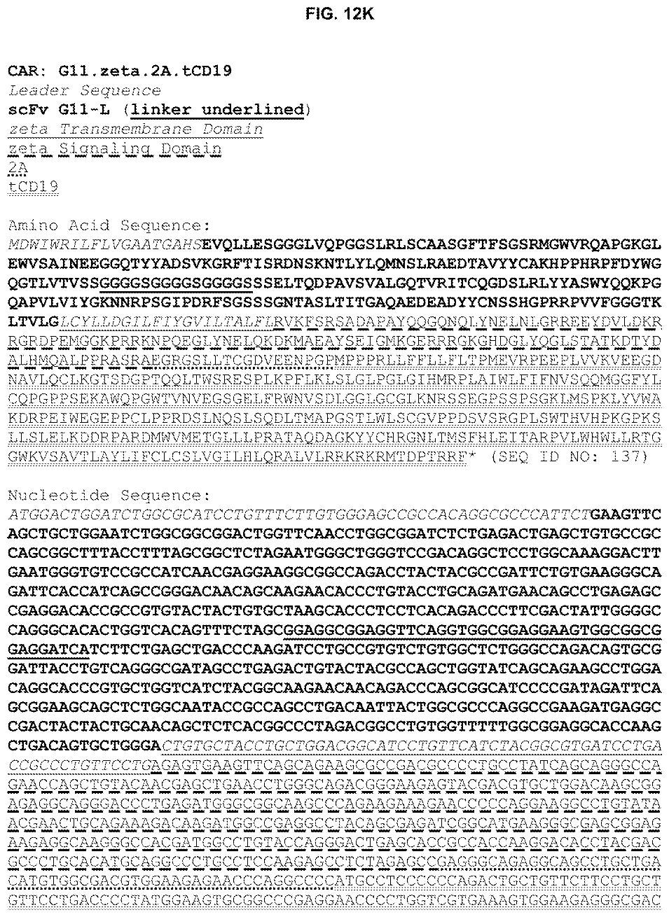

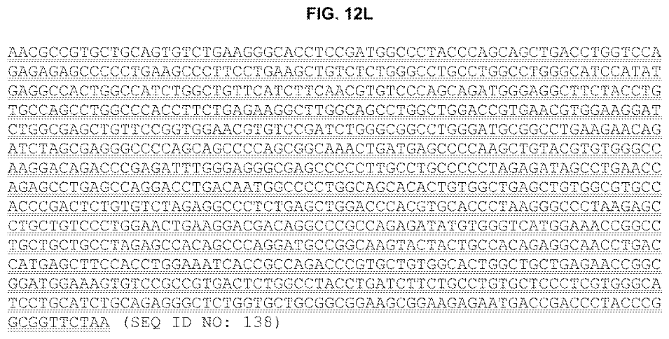

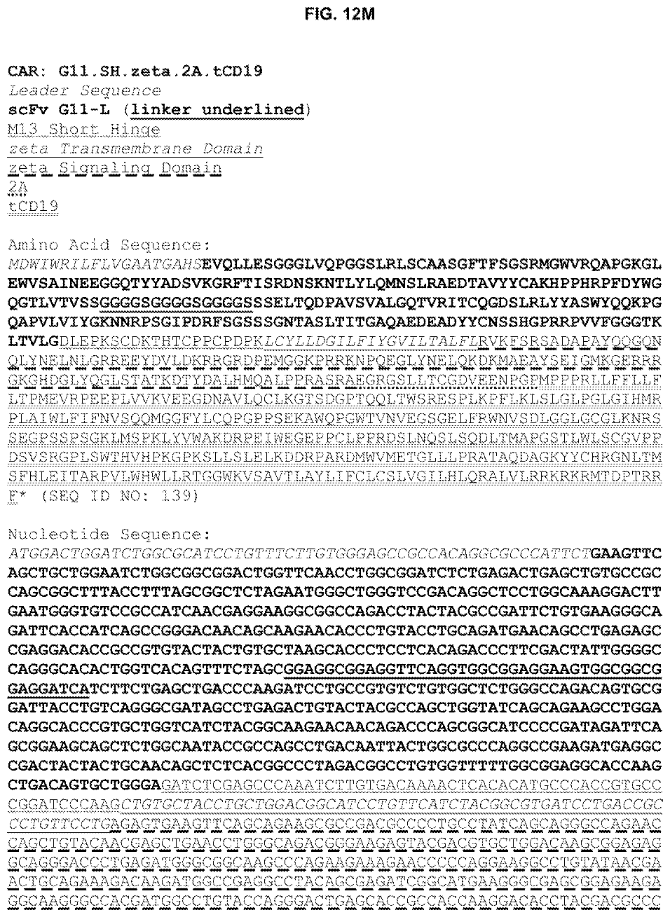

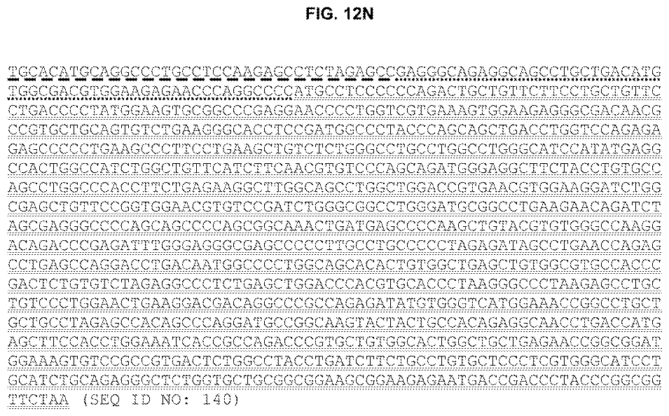

For CAR T cell targeting of C.TNC variant-expressing tumor cells, as schematically represented in FIG. 8A, a retroviral vector was designed encoding a C domain-specific CAR (C.TNC-CAR), utilizing the scFv G11 (see, e.g., U.S. Pat. No. 7,968,685, the content of which is herein incorporated by reference in its entirety), a CD28 hinge/transmembrane domain (CD28H/TM), and a CD28.ζ signaling domain (FIG. 8B). Additional descriptions of C.TNC-CARs of the present disclosure are provided in FIG. 11.

### Example 7: C.TNC-CAR T Cell Recognition and Killing of C.TNC+ Tumor Cells In Vitro

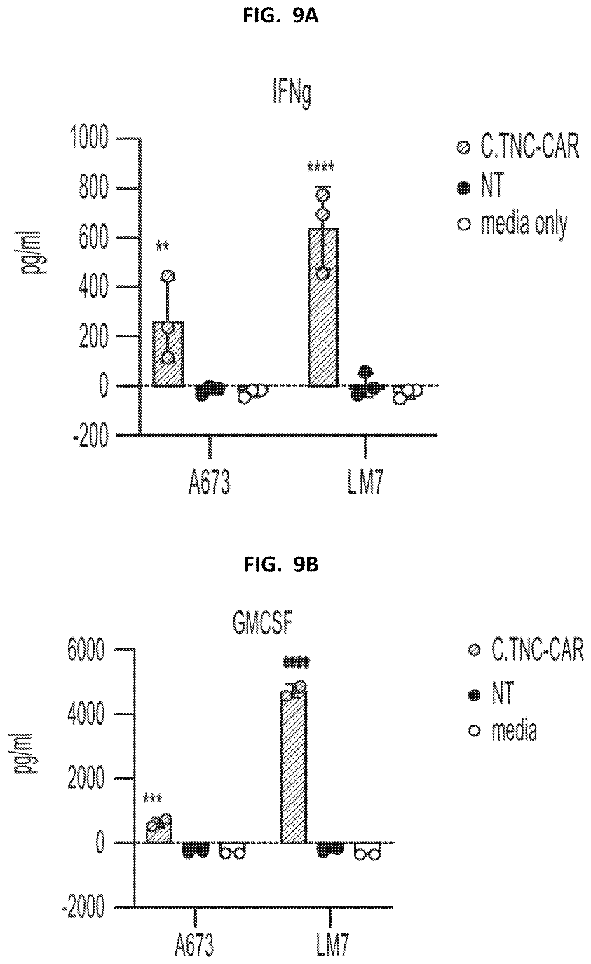

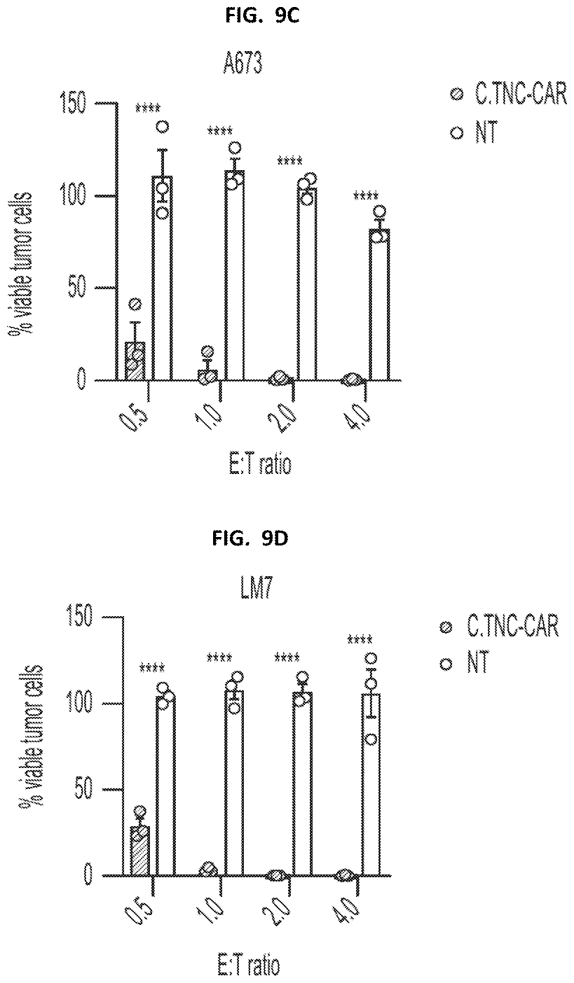

To evaluate C.TNC-CAR T cell recognition and killing of C.TNC+ tumor cells in vitro, multiple cell lines such as, but not limited to, A673 (Ewing's sarcoma) cells, LM7 (osteosarcoma) cells, and non-transduced (NT) T cells, were tested. To measure IFNγ (IFNg) secretion, 5×10⁵ tumor cells were co-cultured with 1×10⁶ T cells. After 48 hours, the cell culture media was harvested, and cytokine production was determined by enzyme-linked immunosorbent assay (ELISA; n=3 donors, **<0.05, ****<0.0001, 2-way ANOVA). The results showed that IFNγ secretion was higher in C.TNC-CAR-T-cell co-cultures than in NT co-culture and media control conditions, across tumor cell types (FIG. 9A).

To measure granulocyte-macrophage colony-stimulating factor (GM-CSF) secretion, 5×10⁵ tumor cells were co-cultured with 1×10⁶ T cells. After 72 hours, the cell culture media was harvested, and cytokine production was determined by ELISA (n=2 donors, **<0.05, ****<0.0001, 2-way ANOVA). The results showed that GM-CSF secretion was higher in G11-CAR-T-cell co-cultures than in NT co-culture and media control conditions, across tumor cell types (FIG. 9B).

Cytolytic activity of C.TNC-CAR T cells was determined by evaluating luminescence produced by firefly luciferase (fflu)-expressing A673.ffluc tumor cells (n=3 donors, ****<0.0001, 2-way ANOVA) and LM7.ffluc tumor cells (n=3 donors, ****<0.0001, 2-way ANOVA) 72 hours post co-culturing T cells and tumor cells. For both A673.ffluc tumor cells (FIG. 9C) and LM7.ffluc tumor cells (FIG. 9D), luminescence units were lower for C.TNC-CAR T cell co-cultures as compared to NT co-cultures regardless of E:T ratio, indicating cytolytic activity of C.TNC-CAR T cells.

### Example 8: C.TNC-CAR T Cells Killing of C.TNC+A673 Cells In Vivo

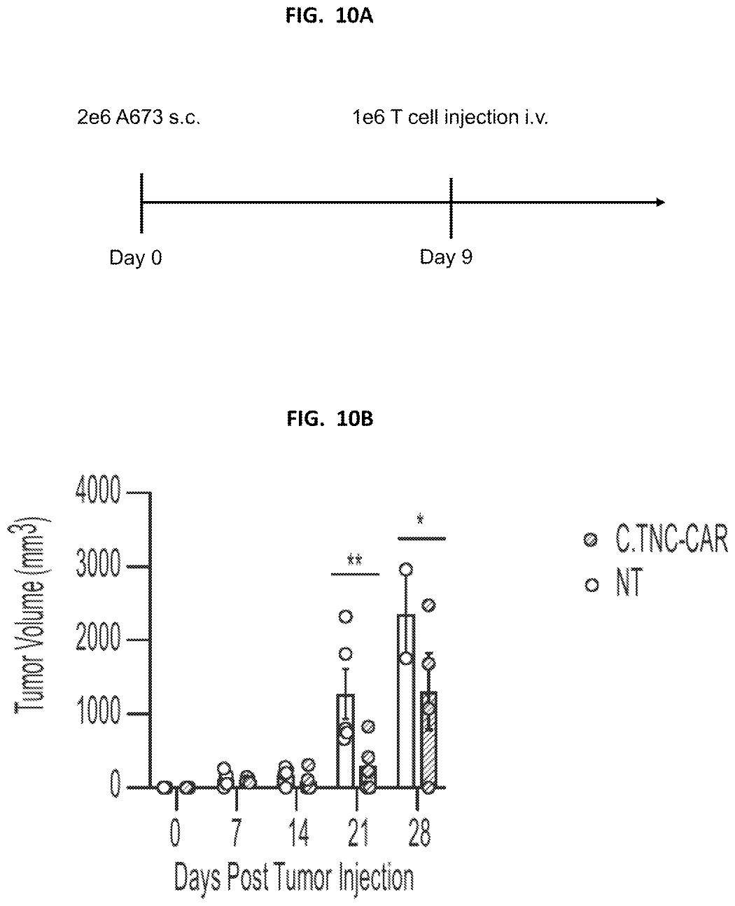

To investigate C.TNC-CAR T cell recognition and killing of C.TNC+ tumor cells in vivo A673 Ewing's sarcoma cells (2×10⁶ cells) were injected subcutaneously (s.c.) into immunodeficient NOD scid gamma (NSG) mice, and on day 9, mice received a single intravenous injection of 1×10⁶ sorted T cells expressing firefly luciferase (ffluc). Mice received C.TNC-CAR T cells or non-transduced (NT) T-cells. A schematic of the experimental setup is presented in FIG. 10A. Tumor growth was measured (mm³) by serial caliper, as shown in FIG. 10B (n=5 mice, *<0.05, **<0.01, 2-way ANOVA). These data demonstrated decreased tumor volume with intravenous injection of C.TNC-CAR T cells.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A polynucleotide encoding a chimeric antigen receptor (CAR) comprising:
(a) an extracellular target-binding domain comprising a binding moiety which binds to procollagen 11A1 (Col11A1), and optionally further comprising a leader sequence and/or a hinge domain,
(b) a transmembrane domain, and
(c) a cytoplasmic domain comprising a signaling domain, and optionally further comprising one or more costimulatory domains.

2-4. (canceled)

5. The polynucleotide of claim 1, wherein the Col11A1 binding moiety comprises an anti-Col11A1 antibody single chain variable fragment (scFv) which binds to an epitope of Col11A1 encoded by exon 6.

6. (canceled)

7. The polynucleotide of claim 1, wherein the Col11A1 binding moiety comprises
(1) a heavy chain complementarity determining region 1 (HCDR1), a HCDR2, and a HCDR3 contained within the heavy chain variable domain (VH) comprising the amino acid sequence SEQ ID NO: 64, or an amino acid sequence having at least 80% identity thereto; and/or a light chain complementarity determining region 1 (LCDR1), a LCDR2, and a LCDR3 contained within the light chain variable domain (VL) comprising the amino acid sequence SEQ ID NO: 68, or an amino acid sequence having at least 80% identity thereto; and/or
(2) a HCDR1 comprising the amino acid sequence of SEQ ID NO: 114, a HCDR2 comprising the amino acid sequence of SEQ ID NO: 115, and a HCDR3 comprising the amino acid sequence of SEQ ID NO: 116; and/or
(3) a LCDR1 comprising the amino acid sequence of SEQ ID NO: 117, a LCDR2 comprising the amino acid sequence of YTS, and a LCDR3 comprising the amino acid sequence SEQ ID NO: 118; and/or
(4) a VH comprising the amino acid sequence SEQ ID NO: 64, or an amino acid sequence having at least 80% identity thereto; and/or
(5) a VL comprising the amino acid sequence SEQ ID NO: 68, or an amino acid sequence having at least 80% identity thereto.

8-9. (canceled)

10. The polynucleotide of claim 7, wherein the polynucleotide comprises the nucleotide sequence SEQ ID NO: 65, or a nucleotide sequence having at least 80% identity thereto; and/or the nucleotide sequence SEQ ID NO: 69, or a nucleotide sequence having at least 80% identity thereto.

11-14. (canceled)

15. The polynucleotide of claim 1, wherein the Col11A1 binding moiety comprises the amino acid sequence SEQ ID NO: 4, or an amino acid sequence having at least 80% identity thereto.

16. The polynucleotide of claim 15, wherein the polynucleotide comprises the nucleotide sequence SEQ ID NO: 5, or a nucleotide sequence having at least 80% identity thereto.

17. (canceled)

18. The polynucleotide of claim 1, wherein the extracellular target-binding domain comprises a leader sequence comprising the amino acid sequence SEQ ID NO: 1, or an amino acid sequence having at least 80% identity thereto.

19. The polynucleotide of claim 18, wherein the polynucleotide comprises the nucleotide sequence SEQ ID NO: 2 or SEQ ID NO: 3, or a nucleotide sequence having at least 80% identity thereto.

20. (canceled)

21. The polynucleotide of claim 1, wherein the extracellular target-binding domain comprises a hinge domain derived from IgG1, IgG2, IgG3, IgG4, CD28, or CD8α.

22. The polynucleotide of claim 21, wherein the hinge domain is derived from IgG1 and comprises the amino acid sequence SEQ ID NO: 15, or an amino acid sequence having at least 80% identity thereto.

23. The polynucleotide of claim 22, wherein the polynucleotide comprises the nucleotide sequence SEQ ID NO: 16, or a nucleotide sequence having at least 80% identity thereto.

24-25. (canceled)

26. The polynucleotide of claim 1, wherein the transmembrane domain is derived from CD28, CD8α, CD4, or CD3ζ.

27. The polynucleotide of claim 26, wherein the transmembrane domain is derived from CD28 and comprises the amino acid sequence SEQ ID NO: 21, or an amino acid sequence having at least 80% identity thereto.

28. The polynucleotide of claim 27, wherein the polynucleotide comprises the nucleotide sequence SEQ ID NO: 22, or a nucleotide sequence having at least 80% identity thereto.

29. The polynucleotide of claim 1, wherein the signaling domain is derived from CD3ζ, DAP10, DAP12, Fc epsilon receptor I γ chain (FCER1G), CD3δ, CD3ε, CD3γ, CD226, NKG2D, or CD79A.

30. The polynucleotide of claim 29, wherein the signaling domain is derived from CD3ζ and comprises the amino acid sequence SEQ ID NO: 29, or an amino acid sequence having at least 80% identity thereto.

31. The polynucleotide of claim 30, wherein the polynucleotide comprises the nucleotide sequence SEQ ID NO: 30, or a nucleotide sequence having at least 80% identity thereto.

32. (canceled)

33. The polynucleotide of claim 1, wherein the cytoplasmic domain comprises one or more costimulatory domains each independently derived from CD28, CD27, CD40, CD134, CD137, CD226, CD79A, ICOS, MyD88, IL-2Rβ, or the STAT3-binding YXXQ.

34. The polynucleotide of claim 33, wherein the costimulatory domain is derived from CD28 and comprises the amino acid sequence SEQ ID NO: 27, or an amino acid sequence having at least 80% identity thereto.

35. The polynucleotide of claim 34, wherein the polynucleotide comprises the nucleotide sequence SEQ ID NO: 28, or a nucleotide sequence having at least 80% identity thereto.

36-48. (canceled)

49. The polynucleotide of claim 1, wherein the CAR comprises the amino acid sequence SEQ ID NO: 52, or an amino acid sequence having at least 80% identity thereto.

50. The polynucleotide of claim 49, wherein the polynucleotide comprises the nucleotide sequence SEQ ID NO: 53, or a nucleotide sequence having at least 80% identity thereto.

51-52. (canceled)

53. A recombinant vector comprising the polynucleotide of claim 1.

54-57. (canceled)

58. A chimeric antigen receptor (CAR) encoded by the polynucleotide of claim 1.

59. An isolated host cell comprising the polynucleotide of claim 1, a recombinant vector comprising the polynucleotide, or a CAR encoded by the polynucleotide.

60-70. (canceled)

71. A pharmaceutical composition comprising the host cell of claim 59 and a pharmaceutically acceptable carrier and/or excipient.

72. A method of generating the isolated host cell of claim 59, said method comprising genetically modifying the host cell with the polynucleotide or a recombinant vector comprising the polynucleotide.

73-75. (canceled)

76. A method for killing a tumor cell expressing Col11A1, said method comprising contacting said cell with the host cell(s) of claim 59 or a pharmaceutical composition comprising said host cell(s).

77. A method for treating a tumor in a subject in need thereof, wherein one or more cells of the tumor express Col11A1, said method comprising administering to the subject a therapeutically effective amount of the host cells of claim 59 or a pharmaceutical composition comprising said host cells.

78-84. (canceled)

85. A polynucleotide encoding a chimeric antigen receptor (CAR) comprising:
(a) an extracellular target-binding domain comprising a binding moiety which binds to a C domain of tenascin C (C.TNC) splice variant,
(b) a transmembrane domain, and
(c) a cytoplasmic domain comprising a signaling domain.

86-147. (canceled)

148. A recombinant vector comprising the polynucleotide of claim 85.

149-152. (canceled)

153. A chimeric antigen receptor (CAR) encoded by the polynucleotide of claim 85.

154. An isolated host cell comprising the polynucleotide of claim 85, a recombinant vector comprising the polynucleotide, or a CAR encoded by the polynucleotide.

155-165. (canceled)

166. A pharmaceutical composition comprising the host cell of claim 154 and a pharmaceutically acceptable carrier and/or excipient.

167. A method of generating an isolated host cell of claim 154, said method comprising genetically modifying the host cell with the polynucleotide or a recombinant vector comprising the polynucleotide.

168-170. (canceled)

171. A method for killing a tumor cell expressing C.TNC, said method comprising contacting said cell with the host cell(s) of claim 154 or a pharmaceutical composition comprising said host cell(s).

172. A method for treating a tumor in a subject in need thereof, wherein one or more cells of the tumor express C.TNC, said method comprising administering to the subject a therapeutically effective amount of the host cells of claim 154 or a pharmaceutical composition comprising said host cells.

173-193. (canceled)


SEQUENCE LISTING (140 sequences)

SEQ ID NO: 1 - protein, 19 aa, Artificial Sequence
  Description of Artificial Sequence Synthetic peptide
MDWIWRILFLVGAATGAHS

SEQ ID NO: 2 - DNA, 57 bp, Artificial Sequence
  Description of Artificial Sequence Synthetic oligonucleotide
atggactggatctggcgcatcctcttcctcgtcggcgctgctaccggcgctcattct

SEQ ID NO: 3 - DNA, 57 bp, Artificial Sequence
  Description of Artificial Sequence Synthetic oligonucleotide
atggactggatctggcgcatcctgtttcttgtgggagccgccacaggcgcccattct

SEQ ID NO: 4 - protein, 238 aa, Artificial Sequence
  Description of Artificial Sequence Synthetic polypeptide
EVQLQESGPELVKTGSSVKISCKASGYSFTGYYMHWVKQSQGKSLEWIGYINCYNGATSYNRNFKGKATFTVDTSSSTAYMQFNSLTSGDSAVYYCAIWDYEFHVMDYWGQRTSLTVGGGGSGGGGSGGGGSENVLTQSPAIMSASLGEKVTMSCRASSSVNYMYWYQQKSDASPKLWIYYTSNLAPGVPARFSGSGSGNSYSLTISSMEGEDADTYYCQQFTSSPWTFGGGTKLEIK

SEQ ID NO: 5 - DNA, 714 bp, Artificial Sequence
  Description of Artificial Sequence Synthetic polynucleotide
gaagtacaactccaagagagcggcccggaactcgttaagaccggaagcagtgtcaaaataagctgcaaggcttccggctacagctttacggggtactatatgcactgggtcaaacaatctcaggggaaaagccttgaatggatcggctatataaattgctataacggagcgacaagctataatcgaaactttaaaggcaaagcaactttcacggtggatacatcatcttcaacggcatatatgcagtttaactcccttacctcaggagactccgccgtttattattgcgctatatgggattatgaattccatgtaatggactattggggtcaaagaacgtctctgactgtgggcggcggcggatcaggcggcggaggaagcggaggtggggggtccgaaaacgtgctgacccagagcccggcgattatgagcgcgagcctgggcgaaaaagtgaccatgagctgccgcgcgagcagcagcgtgaactatatgtattggtatcagcagaaaagcgatgcgagcccgaaactgtggatttattataccagcaacctggcgccgggcgtgccggcgcgctttagcggcagcggcagcggcaacagctatagcctgaccattagcagcatggaaggcgaagatgcggatacctattattgccagcagtttaccagcagcccgtggacctttggcggcggcaccaaactggaaattaaa

SEQ ID NO: 6 - protein, 242 aa, Artificial Sequence
  Description of Artificial Sequence Synthetic polypeptide
EVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLG

SEQ ID NO: 7 - DNA, 726 bp, Artificial Sequence
  Description of Artificial Sequence Synthetic polynucleotide
gaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctggga

SEQ ID NO: 8 - protein, 232 aa, Artificial Sequence
  Description of Artificial Sequence Synthetic polypeptide
EVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLG

SEQ ID NO: 9 - DNA, 696 bp, Artificial Sequence
  Description of Artificial Sequence Synthetic polynucleotide
gaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctggga

SEQ ID NO: 10 - protein, 15 aa, Artificial Sequence
  Description of Artificial Sequence Synthetic peptide
GGGGSGGGGSGGGGS

SEQ ID NO: 11 - DNA, 45 bp, Artificial Sequence
  Description of Artificial Sequence Synthetic oligonucleotide
ggcggcggcggatcaggcggcggaggaagcggaggtggggggtcc

SEQ ID NO: 12 - DNA, 45 bp, Artificial Sequence
  Description of Artificial Sequence Synthetic oligonucleotide
ggaggcggaggttcaggtggcggaggaagtggcggcggaggatca

SEQ ID NO: 13 - protein, 5 aa, Artificial Sequence
  Description of Artificial Sequence Synthetic peptide
GGGGS

SEQ ID NO: 14 - DNA, 15 bp, Artificial Sequence
  Description of Artificial Sequence Synthetic oligonucleotide
ggaggcggaggttca

SEQ ID NO: 15 - protein, 20 aa, Artificial Sequence
  Description of Artificial Sequence Synthetic peptide
DLEPKSCDKTHTCPPCPDPK

SEQ ID NO: 16 - DNA, 60 bp, Artificial Sequence
  Description of Artificial Sequence Synthetic oligonucleotide
gatctcgagcccaaatcttgtgacaaaactcacacatgcccaccgtgcccggatcccaag

SEQ ID NO: 17 - protein, 231 aa, Artificial Sequence
  Description of Artificial Sequence Synthetic polypeptide
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGS

SEQ ID NO: 18 - DNA, 693 bp, Artificial Sequence
  Description of Artificial Sequence Synthetic polynucleotide
gagtctaagtacggccctccttgtcctagctgccccgctcctgaatttgaaggcggcccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctcggcaagggctcc

SEQ ID NO: 19 - protein, 45 aa, Artificial Sequence
  Description of Artificial Sequence Synthetic polypeptide
TTTPAPRPPTPAPTIASQPLSLRPEACRPAAGGAVHTRGLDFACD

SEQ ID NO: 20 - DNA, 135 bp, Artificial Sequence
  Description of Artificial Sequence Synthetic polynucleotide
accaccacaccagctcctcggcctccaactcctgctcctacaatagccagccagcctctgtctctgaggcccgaagcatgcagacctgctgctggcggagccgtgcatacaagaggactggatttcgcctgcgac

SEQ ID NO: 21 - protein, 27 aa, Artificial Sequence
  Description of Artificial Sequence Synthetic peptide
FWVLVVVGGVLACYSLLVTVAFIIFWV

SEQ ID NO: 22 - DNA, 81 bp, Artificial Sequence
  Description of Artificial Sequence Synthetic oligonucleotide
ttctgggtgctggtggtcgtgggcggagtgctggcctgttacagcctgctcgtgaccgtggccttcatcatcttttgggtg

SEQ ID NO: 23 - protein, 24 aa, Artificial Sequence
  Description of Artificial Sequence Synthetic peptide
IYIWAPLAGTCGVLLLSLVITLYC

SEQ ID NO: 24 - DNA, 72 bp, Artificial Sequence
  Description of Artificial Sequence Synthetic oligonucleotide
atctacatctgggcccctctggctggaacatgtggcgttctgctgctgagcctggtcatcaccctgtactgc

SEQ ID NO: 25 - protein, 21 aa, Artificial Sequence
  Description of Artificial Sequence Synthetic peptide
LCYLLDGILFIYGVILTALFL

SEQ ID NO: 26 - DNA, 63 bp, Artificial Sequence
  Description of Artificial Sequence Synthetic oligonucleotide
ctgtgctacctgctggacggcatcctgttcatctacggcgtgatcctgaccgccctgttcctg

SEQ ID NO: 27 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 28 - DNA, 123 bp, Artificial Sequence
  Description of Artificial Sequence Synthetic polynucleotide
cgcagcaagcggagccggctgctgcacagcgactacatgaacatgacccccagacggcctggccccaccagaaagcactaccagccttacgcccctcccagagacttcgccgcctaccggtcc

SEQ ID NO: 29 - protein, 116 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRA

SEQ ID NO: 30 - DNA, 348 bp, Artificial Sequence
  Description of Artificial Sequence Synthetic polynucleotide
agagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagcc

SEQ ID NO: 31 - protein, 18 aa, Thosea asigna virus
EGRGSLLTCGDVEENPGP

SEQ ID NO: 32 - DNA, 54 bp, Thosea asigna virus
gagggcagaggcagcctgctgacatgtggcgacgtggaagagaacccaggcccc

SEQ ID NO: 33 - protein, 333 aa, Artificial Sequence
  Description of Artificial Sequence Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 34 - DNA, 999 bp, Artificial Sequence
  Description of Artificial Sequence Synthetic polynucleotide
atgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttc

SEQ ID NO: 35 - DNA, 1002 bp, Artificial Sequence
  Description of Artificial Sequence Synthetic polynucleotide
atgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 36 - protein, 277 aa, Artificial Sequence
  Description of Artificial Sequence Synthetic polypeptide
MDWIWRILFLVGAATGAHSEVQLQESGPELVKTGSSVKISCKASGYSFTGYYMHWVKQSQGKSLEWIGYINCYNGATSYNRNFKGKATFTVDTSSSTAYMQFNSLTSGDSAVYYCAIWDYEFHVMDYWGQRTSLTVGGGGSGGGGSGGGGSENVLTQSPAIMSASLGEKVTMSCRASSSVNYMYWYQQKSDASPKLWIYYTSNLAPGVPARFSGSGSGNSYSLTISSMEGEDADTYYCQQFTSSPWTFGGGTKLEIKDLEPKSCDKTHTCPPCPDPK

SEQ ID NO: 37 - DNA, 831 bp, Artificial Sequence
  Description of Artificial Sequence Synthetic polynucleotide
atggactggatctggcgcatcctcttcctcgtcggcgctgctaccggcgctcattctgaagtacaactccaagagagcggcccggaactcgttaagaccggaagcagtgtcaaaataagctgcaaggcttccggctacagctttacggggtactatatgcactgggtcaaacaatctcaggggaaaagccttgaatggatcggctatataaattgctataacggagcgacaagctataatcgaaactttaaaggcaaagcaactttcacggtggatacatcatcttcaacggcatatatgcagtttaactcccttacctcaggagactccgccgtttattattgcgctatatgggattatgaattccatgtaatggactattggggtcaaagaacgtctctgactgtgggcggcggcggatcaggcggcggaggaagcggaggtggggggtccgaaaacgtgctgacccagagcccggcgattatgagcgcgagcctgggcgaaaaagtgaccatgagctgccgcgcgagcagcagcgtgaactatatgtattggtatcagcagaaaagcgatgcgagcccgaaactgtggatttattataccagcaacctggcgccgggcgtgccggcgcgctttagcggcagcggcagcggcaacagctatagcctgaccattagcagcatggaaggcgaagatgcggatacctattattgccagcagtttaccagcagcccgtggacctttggcggcggcaccaaactggaaattaaagatctcgagcccaaatcttgtgacaaaactcacacatgcccaccgtgcccggatcccaag

SEQ ID NO: 38 - protein, 281 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGDLEPKSCDKTHTCPPCPDPK

SEQ ID NO: 39 - DNA, 843 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggagatctcgagcccaaatcttgtgacaaaactcacacatgcccaccgtgcccggatcccaag

SEQ ID NO: 40 - protein, 271 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGDLEPKSCDKTHTCPPCPDPK

SEQ ID NO: 41 - DNA, 813 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggagatctcgagcccaaatcttgtgacaaaactcacacatgcccaccgtgcccggatcccaag

SEQ ID NO: 42 - protein, 492 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGS

SEQ ID NO: 43 - DNA, 1476 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggagagtctaagtacggccctccttgtcctagctgccccgctcctgaatttgaaggcggcccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctcggcaagggctcc

SEQ ID NO: 44 - protein, 330 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGTTTPAPRPPTPAPTIASQPLSLRPEACRPAAGGAVHTRGLDFACDIYIWAPLAGTCGVLLLSLVITLYC

SEQ ID NO: 45 - DNA, 990 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggaaccaccacaccagctcctcggcctccaactcctgctcctacaatagccagccagcctctgtctctgaggcccgaagcatgcagacctgctgctggcggagccgtgcatacaagaggactggatttcgcctgcgacatctacatctgggcccctctggctggaacatgtggcgttctgctgctgagcctggtcatcaccctgtactgc

SEQ ID NO: 46 - protein, 261 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLG

SEQ ID NO: 47 - DNA, 783 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctggga

SEQ ID NO: 48 - protein, 157 aa, Artificial Sequence
  Description of Artificial Sequence Synthetic polypeptide
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRA

SEQ ID NO: 49 - DNA, 471 bp, Artificial Sequence
  Description of Artificial Sequence Synthetic polynucleotide
cgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagcc

SEQ ID NO: 50 - protein, 116 aa, Artificial Sequence; identical to SEQ ID NO: 29
  Description of Artificial Sequence Synthetic polypeptide

SEQ ID NO: 51 - DNA, 348 bp, Artificial Sequence; identical to SEQ ID NO: 30
  Description of Artificial Sequence Synthetic polynucleotide

SEQ ID NO: 52 - protein, 461 aa, Artificial Sequence
  Description of Artificial Sequence Synthetic polypeptide
MDWIWRILFLVGAATGAHSEVQLQESGPELVKTGSSVKISCKASGYSFTGYYMHWVKQSQGKSLEWIGYINCYNGATSYNRNFKGKATFTVDTSSSTAYMQFNSLTSGDSAVYYCAIWDYEFHVMDYWGQRTSLTVGGGGSGGGGSGGGGSENVLTQSPAIMSASLGEKVTMSCRASSSVNYMYWYQQKSDASPKLWIYYTSNLAPGVPARFSGSGSGNSYSLTISSMEGEDADTYYCQQFTSSPWTFGGGTKLEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRA

SEQ ID NO: 53 - DNA, 1383 bp, Artificial Sequence
  Description of Artificial Sequence Synthetic polynucleotide
atggactggatctggcgcatcctcttcctcgtcggcgctgctaccggcgctcattctgaagtacaactccaagagagcggcccggaactcgttaagaccggaagcagtgtcaaaataagctgcaaggcttccggctacagctttacggggtactatatgcactgggtcaaacaatctcaggggaaaagccttgaatggatcggctatataaattgctataacggagcgacaagctataatcgaaactttaaaggcaaagcaactttcacggtggatacatcatcttcaacggcatatatgcagtttaactcccttacctcaggagactccgccgtttattattgcgctatatgggattatgaattccatgtaatggactattggggtcaaagaacgtctctgactgtgggcggcggcggatcaggcggcggaggaagcggaggtggggggtccgaaaacgtgctgacccagagcccggcgattatgagcgcgagcctgggcgaaaaagtgaccatgagctgccgcgcgagcagcagcgtgaactatatgtattggtatcagcagaaaagcgatgcgagcccgaaactgtggatttattataccagcaacctggcgccgggcgtgccggcgcgctttagcggcagcggcagcggcaacagctatagcctgaccattagcagcatggaaggcgaagatgcggatacctattattgccagcagtttaccagcagcccgtggacctttggcggcggcaccaaactggaaattaaagatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagcc

SEQ ID NO: 54 - protein, 465 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRA

SEQ ID NO: 55 - DNA, 1395 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggagatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagcc

SEQ ID NO: 56 - protein, 455 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRA

SEQ ID NO: 57 - DNA, 1365 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggagatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagcc

SEQ ID NO: 58 - protein, 676 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRA

SEQ ID NO: 59 - DNA, 2028 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggagagtctaagtacggccctccttgtcctagctgccccgctcctgaatttgaaggcggcccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctcggcaagggctccttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagcc

SEQ ID NO: 60 - protein, 487 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRA

SEQ ID NO: 61 - DNA, 1461 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggaaccaccacaccagctcctcggcctccaactcctgctcctacaatagccagccagcctctgtctctgaggcccgaagcatgcagacctgctgctggcggagccgtgcatacaagaggactggatttcgcctgcgacatctacatctgggcccctctggctggaacatgtggcgttctgctgctgagcctggtcatcaccctgtactgccgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagcc

SEQ ID NO: 62 - protein, 398 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRA

SEQ ID NO: 63 - DNA, 1194 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggactgtgctacctgctggacggcatcctgttcatctacggcgtgatcctgaccgccctgttcctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagcc

SEQ ID NO: 64 - protein, 117 aa, Artificial Sequence
  Description of Artificial Sequence Synthetic polypeptide
EVQLQESGPELVKTGSSVKISCKASGYSFTGYYMHWVKQSQGKSLEWIGYINCYNGATSYNRNFKGKATFTVDTSSSTAYMQFNSLTSGDSAVYYCAIWDYEFHVMDYWGQRTSLTV

SEQ ID NO: 65 - DNA, 351 bp, Artificial Sequence
  Description of Artificial Sequence Synthetic polynucleotide
gaagtacaactccaagagagcggcccggaactcgttaagaccggaagcagtgtcaaaataagctgcaaggcttccggctacagctttacggggtactatatgcactgggtcaaacaatctcaggggaaaagccttgaatggatcggctatataaattgctataacggagcgacaagctataatcgaaactttaaaggcaaagcaactttcacggtggatacatcatcttcaacggcatatatgcagtttaactcccttacctcaggagactccgccgtttattattgcgctatatgggattatgaattccatgtaatggactattggggtcaaagaacgtctctgactgtg

SEQ ID NO: 66 - protein, 118 aa, Artificial Sequence
  Description of Artificial Sequence Synthetic polypeptide
EVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSS

SEQ ID NO: 67 - DNA, 354 bp, Artificial Sequence
  Description of Artificial Sequence Synthetic polynucleotide
gaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagc

SEQ ID NO: 68 - protein, 106 aa, Artificial Sequence
  Description of Artificial Sequence Synthetic polypeptide
ENVLTQSPAIMSASLGEKVTMSCRASSSVNYMYWYQQKSDASPKLWIYYTSNLAPGVPARFSGSGSGNSYSLTISSMEGEDADTYYCQQFTSSPWTFGGGTKLEIK

SEQ ID NO: 69 - DNA, 318 bp, Artificial Sequence
  Description of Artificial Sequence Synthetic polynucleotide
gaaaacgtgctgacccagagcccggcgattatgagcgcgagcctgggcgaaaaagtgaccatgagctgccgcgcgagcagcagcgtgaactatatgtattggtatcagcagaaaagcgatgcgagcccgaaactgtggatttattataccagcaacctggcgccgggcgtgccggcgcgctttagcggcagcggcagcggcaacagctatagcctgaccattagcagcatggaaggcgaagatgcggatacctattattgccagcagtttaccagcagcccgtggacctttggcggcggcaccaaactggaaattaaa

SEQ ID NO: 70 - protein, 109 aa, Artificial Sequence
  Description of Artificial Sequence Synthetic polypeptide
SSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLG

SEQ ID NO: 71 - DNA, 327 bp, Artificial Sequence
  Description of Artificial Sequence Synthetic polynucleotide
tcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctggga

SEQ ID NO: 72 - protein, 10 aa, Artificial Sequence
  Description of Artificial Sequence Synthetic peptide
GGGGSGGGGS

SEQ ID NO: 73 - protein, 20 aa, Artificial Sequence
  Description of Artificial Sequence Synthetic peptide
GGGGSGGGGSGGGGSGGGGS

SEQ ID NO: 74 - protein, 18 aa, Artificial Sequence
  Description of Artificial Sequence Synthetic peptide
KESGSVSSEQLAQFRSLD

SEQ ID NO: 75 - protein, 14 aa, Artificial Sequence
  Description of Artificial Sequence Synthetic peptide
EGKSSGSGSESKST

SEQ ID NO: 76 - protein, 15 aa, Artificial Sequence
  Description of Artificial Sequence Synthetic peptide
EGKSSGSGSESKSTQ

SEQ ID NO: 77 - protein, 14 aa, Artificial Sequence
  Description of Artificial Sequence Synthetic peptide
GSTSGSGKSSEGKG

SEQ ID NO: 78 - protein, 25 aa, Artificial Sequence
  Description of Artificial Sequence Synthetic peptide
SSADDAKKDDAKKDDAKKDDAKKDG

SEQ ID NO: 79 - protein, 14 aa, Artificial Sequence
  Description of Artificial Sequence Synthetic peptide
EGKSSGSGSESKVD

SEQ ID NO: 80 - protein, 16 aa, Artificial Sequence
  Description of Artificial Sequence Synthetic peptide
ESGSVSSEELAFRSLD

SEQ ID NO: 81 - protein, 14 aa, Artificial Sequence
  Description of Artificial Sequence Synthetic peptide
EGKSSGSGSESKST

SEQ ID NO: 82 - protein, 15 aa, Artificial Sequence
  Description of Artificial Sequence Synthetic peptide
EGKSSGSGSESKSTQ

SEQ ID NO: 83 - protein, 15 aa, Artificial Sequence
  Description of Artificial Sequence Synthetic peptide
EPKSCDKTHTCPPCP

SEQ ID NO: 84 - protein, 12 aa, Artificial Sequence
  Description of Artificial Sequence Synthetic peptide
ERKCCVECPPCP

SEQ ID NO: 85 - protein, 62 aa, Artificial Sequence
  Description of Artificial Sequence Synthetic polypeptide
ELKTPLGDTTHTCPRCPEPKSCDTPPPCPRCPEPKSCDTPPPCPRCPEPKSCDTPPPCPRCP

SEQ ID NO: 86 - protein, 12 aa, Artificial Sequence
  Description of Artificial Sequence Synthetic peptide
ESKYGPPCPSCP

SEQ ID NO: 87 - protein, 19 aa, Thosea asigna virus
AEGRGSLLTCGDVEENPGP

SEQ ID NO: 88 - protein, 21 aa, Artificial Sequence
  Description of Artificial Sequence Synthetic peptide
GSGEGRGSLLTCGDVEENPGP

SEQ ID NO: 89 - protein, 63 aa, Artificial Sequence
  Description of Artificial Sequence Synthetic polypeptide
GSGSRVTELLYRMKRAETYCPRPLLAIHPTEARHKQKIVAPVKQLLNFDLLKLAGDVESNPGP

SEQ ID NO: 90 - protein, 19 aa, Amphimedon queenslandica
LLCFLLLLLSGDVELNPGP

SEQ ID NO: 91 - protein, 20 aa, Amphimedon queenslandica
HHFMFLLLLLAGDIELNPGP

SEQ ID NO: 92 - protein, 20 aa, Saccoglossus kowalevskii
WFLVLLSFILSGDIEVNPGP

SEQ ID NO: 93 - protein, 20 aa, Branchiostoma floridae
KNCAMYMLLLSGDVETNPGP

SEQ ID NO: 94 - protein, 20 aa, Branchiostoma floridae
MVISQLMLKLAGDVEENPGP

SEQ ID NO: 95 - protein, 22 aa, Artificial Sequence
  Description of Artificial Sequence Synthetic peptide
GSGATNFSLLKQAGDVEENPGP

SEQ ID NO: 96 - protein, 23 aa, Artificial Sequence
  Description of Artificial Sequence Synthetic peptide
GSGQCTNYALLKLAGDVESNPGP

SEQ ID NO: 97 - protein, 8 aa, Artificial Sequence
  Description of Artificial Sequence Synthetic peptide
  MOD_RES (2)..(2): Any amino acid
  MOD_RES (4)..(4): Any amino acid
DXEXNPGP

SEQ ID NO: 98 - protein, 21 aa, Artificial Sequence
  Description of Artificial Sequence Synthetic peptide
MALPVTALLLPLALLLHAARP

SEQ ID NO: 99 - DNA, 63 bp, Artificial Sequence
  Description of Artificial Sequence Synthetic oligonucleotide
atggccttaccagtgaccgccttgctcctgccgctggccttgctgctccacgccgccaggccg

SEQ ID NO: 100 - protein, 39 aa, Artificial Sequence
  Description of Artificial Sequence Synthetic polypeptide
IEVMYPPPYLDNEKSNGTIIHVKGKHLCPSPLFPGPSKP

SEQ ID NO: 101 - DNA, 116 bp, Artificial Sequence
  Description of Artificial Sequence Synthetic polynucleotide
atcgaagtgatgtacccgcctccttacctggacaacgagaagtccaacggcaccatcatccacgtgaagggaaagcacctgtgtccttctccactgttccccggacctagcaagcc

SEQ ID NO: 102 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 103 - DNA, 126 bp, Artificial Sequence
  Description of Artificial Sequence Synthetic polynucleotide
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 104 - protein, 36 aa, Artificial Sequence
  Description of Artificial Sequence Synthetic polypeptide
RDQRLPPDAHKPPGGGSFRTPIQEEQADAHSTLAKI

SEQ ID NO: 105 - DNA, 108 bp, Artificial Sequence
  Description of Artificial Sequence Synthetic polynucleotide
agggaccagaggctgccccccgatgcccacaagccccctgggggaggcagtttccggacccccatccaagaggagcaggccgacgcccactccaccctggccaagatc

SEQ ID NO: 106 - protein, 171 aa, Artificial Sequence
  Description of Artificial Sequence Synthetic polypeptide
AAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDI

SEQ ID NO: 107 - DNA, 513 bp, Artificial Sequence
  Description of Artificial Sequence Synthetic polynucleotide
gctgctggcggacctggcgccggatctgctgctcctgtgtctagcacaagcagcctgcctctggccgccctgaacatgagagtgcggagaaggctgagcctgttcctgaacgtgcggacacaggtggccgccgattggacagccctggccgaggaaatggacttcgagtacctggaaatccggcagctggaaacccaggccgaccctacaggcagactgctggatgcttggcagggcagaccaggcgcttctgtgggaaggctgctggaactgctgaccaagctgggcagggacgacgtgctgctggaactgggccctagcatcgaagaggactgccagaagtacatcctgaagcagcagcaggaagaggccgagaagcctctgcaggtggcagccgtggatagcagcgtgccaagaacagccgagctggccggcatcaccaccctggatgatcctctgggccacatgcccgagagattcgacgccttcatctgctactgccccagcgacatc

SEQ ID NO: 108 - protein, 172 aa, Artificial Sequence
  Description of Artificial Sequence Synthetic polypeptide
MAAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 109 - DNA, 516 bp, Artificial Sequence
  Description of Artificial Sequence Synthetic polynucleotide
atggccgctgggggcccaggcgccggatcagctgctcccgtatcttctacttcttctttgccgctggctgctctgaacatgcgcgtgagaagacgcctctccctgttccttaacgttcgcacacaagtcgctgccgattggaccgcccttgccgaagaaatggactttgaatacctggaaattagacaacttgaaacacaggccgaccccactggcagactcctggacgcatggcagggaagacctggtgcaagcgttggacggctcctggatctcctgacaaaactgggacgcgacgacgtactgcttgaactcggacctagcattgaagaagactgccaaaaatatatcctgaaacaacaacaagaagaagccgaaaaacctctccaagtcgcagcagtggactcatcagtaccccgaacagctgagcttgctgggattactacactcgacgacccactcggacatatgcctgaaagattcgacgctttcatttgctattgcccctctgacata

SEQ ID NO: 110 - protein, 171 aa, Artificial Sequence
  Description of Artificial Sequence Synthetic polypeptide
AAGGPGAGSAAPVSSTSSLPLAALNMRVRRRLSLFLNVRTQVAADWTALAEEMDFEYLEIRQLETQADPTGRLLDAWQGRPGASVGRLLDLLTKLGRDDVLLELGPSIEEDCQKYILKQQQEEAEKPLQVAAVDSSVPRTAELAGITTLDDPLGHMPERFDAFICYCPSDI

SEQ ID NO: 111 - DNA, 513 bp, Artificial Sequence
  Description of Artificial Sequence Synthetic polynucleotide
gccgctgggggcccaggcgccggatcagctgctcccgtatcttctacttcttctttgccgctggctgctctgaacatgcgcgtgagaagacgcctctccctgttccttaacgttcgcacacaagtcgctgccgattggaccgcccttgccgaagaaatggactttgaatacctggaaattagacaacttgaaacacaggccgaccccactggcagactcctggacgcatggcagggaagacctggtgcaagcgttggacggctcctggatctcctgacaaaactgggacgcgacgacgtactgcttgaactcggacctagcattgaagaagactgccaaaaatatatcctgaaacaacaacaagaagaagccgaaaaacctctccaagtcgcagcagtggactcatcagtaccccgaacagctgagcttgctgggattactacactcgacgacccactcggacatatgcctgaaagattcgacgctttcatttgctattgcccctctgacata

SEQ ID NO: 112 - protein, 62 aa, Artificial Sequence
  Description of Artificial Sequence Synthetic polypeptide
KKVAKKPTNKAPHPKQEPQEINFPDDLPGSNTAAPVQETLHGCQPVTQEDGKESRISVQERQ

SEQ ID NO: 113 - DNA, 186 bp, Artificial Sequence
  Description of Artificial Sequence Synthetic polynucleotide
aagaaggtggccaagaagcccaccaacaaggccccccaccccaagcaggaaccccaggaaatcaacttccccgacgacctgcccggcagcaatactgctgcacccgtgcaggaaaccctgcacggctgtcagcctgtgacccaggaagatggcaaagaaagccggatctctgtgcaggaacgccag

SEQ ID NO: 114 - protein, 8 aa, Artificial Sequence
  Description of Artificial Sequence Synthetic peptide
GYSFTGYY

SEQ ID NO: 115 - protein, 8 aa, Artificial Sequence
  Description of Artificial Sequence Synthetic peptide
INCYNGAT

SEQ ID NO: 116 - protein, 12 aa, Artificial Sequence
  Description of Artificial Sequence Synthetic peptide
AIWDYEFHVMDY

SEQ ID NO: 117 - protein, 5 aa, Artificial Sequence
  Description of Artificial Sequence Synthetic peptide
SSVNY

SEQ ID NO: 118 - protein, 9 aa, Artificial Sequence
  Description of Artificial Sequence Synthetic peptide
QQFTSSPWT

SEQ ID NO: 119 - protein, 5 aa, Artificial Sequence
  Description of Artificial Sequence Synthetic peptide
GSRMG

SEQ ID NO: 120 - protein, 16 aa, Artificial Sequence
  Description of Artificial Sequence Synthetic peptide
AINEEGGQTYYADSVK

SEQ ID NO: 121 - protein, 9 aa, Artificial Sequence
  Description of Artificial Sequence Synthetic peptide
HPPHRPFDY

SEQ ID NO: 122 - protein, 11 aa, Artificial Sequence
  Description of Artificial Sequence Synthetic peptide
QGDSLRLYYAS

SEQ ID NO: 123 - protein, 7 aa, Artificial Sequence
  Description of Artificial Sequence Synthetic peptide
GKNNRPS

SEQ ID NO: 124 - protein, 11 aa, Artificial Sequence
  Description of Artificial Sequence Synthetic peptide
NSSHGPRRPVV

SEQ ID NO: 125 - protein, 418 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGDLEPKSCDKTHTCPPCPDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRA

SEQ ID NO: 126 - DNA, 1254 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggagatctcgagcccaaatcttgtgacaaaactcacacatgcccaccgtgcccggatcccaagctgtgctacctgctggacggcatcctgttcatctacggcgtgatcctgaccgccctgttcctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagcc

SEQ ID NO: 127 - protein, 812 aa, Artificial Sequence
  Description of Artificial Sequence Synthetic polypeptide
MDWIWRILFLVGAATGAHSEVQLQESGPELVKTGSSVKISCKASGYSFTGYYMHWVKQSQGKSLEWIGYINCYNGATSYNRNFKGKATFTVDTSSSTAYMQFNSLTSGDSAVYYCAIWDYEFHVMDYWGQRTSLTVGGGGSGGGGSGGGGSENVLTQSPAIMSASLGEKVTMSCRASSSVNYMYWYQQKSDASPKLWIYYTSNLAPGVPARFSGSGSGNSYSLTISSMEGEDADTYYCQQFTSSPWTFGGGTKLEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRAEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 128 - DNA, 2436 bp, Artificial Sequence
  Description of Artificial Sequence Synthetic polynucleotide
atggactggatctggcgcatcctcttcctcgtcggcgctgctaccggcgctcattctgaagtacaactccaagagagcggcccggaactcgttaagaccggaagcagtgtcaaaataagctgcaaggcttccggctacagctttacggggtactatatgcactgggtcaaacaatctcaggggaaaagccttgaatggatcggctatataaattgctataacggagcgacaagctataatcgaaactttaaaggcaaagcaactttcacggtggatacatcatcttcaacggcatatatgcagtttaactcccttacctcaggagactccgccgtttattattgcgctatatgggattatgaattccatgtaatggactattggggtcaaagaacgtctctgactgtgggcggcggcggatcaggcggcggaggaagcggaggtggggggtccgaaaacgtgctgacccagagcccggcgattatgagcgcgagcctgggcgaaaaagtgaccatgagctgccgcgcgagcagcagcgtgaactatatgtattggtatcagcagaaaagcgatgcgagcccgaaactgtggatttattataccagcaacctggcgccgggcgtgccggcgcgctttagcggcagcggcagcggcaacagctatagcctgaccattagcagcatggaaggcgaagatgcggatacctattattgccagcagtttaccagcagcccgtggacctttggcggcggcaccaaactggaaattaaagatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagccgagggcagaggcagcctgctgacatgtggcgacgtggaagagaacccaggccccatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttc

SEQ ID NO: 129 - protein, 816 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRAEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 130 - DNA, 2451 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggagatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagccgagggcagaggcagcctgctgacatgtggcgacgtggaagagaacccaggccccatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 131 - protein, 806 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRAEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 132 - DNA, 2421 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggagatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagccgagggcagaggcagcctgctgacatgtggcgacgtggaagagaacccaggccccatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 133 - protein, 1027 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRAEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 134 - DNA, 3084 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggagagtctaagtacggccctccttgtcctagctgccccgctcctgaatttgaaggcggcccttccgtgttcctgtttcctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttccagagcacctacagagtggtgtccgtgctgacagtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgcagcgtgatgcacgaggccctgcacaaccactacacccagaagtctctgagcctgagcctcggcaagggctccttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagccgagggcagaggcagcctgctgacatgtggcgacgtggaagagaacccaggccccatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 135 - protein, 838 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRAEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 136 - DNA, 2517 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggaaccaccacaccagctcctcggcctccaactcctgctcctacaatagccagccagcctctgtctctgaggcccgaagcatgcagacctgctgctggcggagccgtgcatacaagaggactggatttcgcctgcgacatctacatctgggcccctctggctggaacatgtggcgttctgctgctgagcctggtcatcaccctgtactgccgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagccgagggcagaggcagcctgctgacatgtggcgacgtggaagagaacccaggccccatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 137 - protein, 749 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRAEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 138 - DNA, 2250 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggactgtgctacctgctggacggcatcctgttcatctacggcgtgatcctgaccgccctgttcctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagccgagggcagaggcagcctgctgacatgtggcgacgtggaagagaacccaggccccatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 139 - protein, 769 aa, Artificial Sequence
  Description of Artificial Sequence Synthetic polypeptide
MDWIWRILFLVGAATGAHSEVQLLESGGGLVQPGGSLRLSCAASGFTFSGSRMGWVRQAPGKGLEWVSAINEEGGQTYYADSVKGRFTISRDNSKNTLYLQMNSLRAEDTAVYYCAKHPPHRPFDYWGQGTLVTVSSGGGGSGGGGSGGGGSSSELTQDPAVSVALGQTVRITCQGDSLRLYYASWYQQKPGQAPVLVIYGKNNRPSGIPDRFSGSSSGNTASLTITGAQAEDEADYYCNSSHGPRRPVVFGGGTKLTVLGDLEPKSCDKTHTCPPCPDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRAEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 140 - DNA, 2310 bp, Artificial Sequence
  Description of Artificial Sequence Synthetic polynucleotide
atggactggatctggcgcatcctgtttcttgtgggagccgccacaggcgcccattctgaagttcagctgctggaatctggcggcggactggttcaacctggcggatctctgagactgagctgtgccgccagcggctttacctttagcggctctagaatgggctgggtccgacaggctcctggcaaaggacttgaatgggtgtccgccatcaacgaggaaggcggccagacctactacgccgattctgtgaagggcagattcaccatcagccgggacaacagcaagaacaccctgtacctgcagatgaacagcctgagagccgaggacaccgccgtgtactactgtgctaagcaccctcctcacagacccttcgactattggggccagggcacactggtcacagtttctagcggaggcggaggttcaggtggcggaggaagtggcggcggaggatcatcttctgagctgacccaagatcctgccgtgtctgtggctctgggccagacagtgcggattacctgtcagggcgatagcctgagactgtactacgccagctggtatcagcagaagcctggacaggcacccgtgctggtcatctacggcaagaacaacagacccagcggcatccccgatagattcagcggaagcagctctggcaataccgccagcctgacaattactggcgcccaggccgaagatgaggccgactactactgcaacagctctcacggccctagacggcctgtggtttttggcggaggcaccaagctgacagtgctgggagatctcgagcccaaatcttgtgacaaaactcacacatgcccaccgtgcccggatcccaagctgtgctacctgctggacggcatcctgttcatctacggcgtgatcctgaccgccctgttcctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagccgagggcagaggcagcctgctgacatgtggcgacgtggaagagaacccaggccccatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa